SEQUENCE LISTING (179 sequences)

SEQ ID NO: 1 - protein, 10 aa, Artificial Sequence
  Chemically Synthesized, CD9B366 HCDR1
GFTFSNYWMS

SEQ ID NO: 2 - protein, 10 aa, Artificial Sequence
  Chemically Synthesized, CD9B366 HCDR2
NINQGGSEKY

SEQ ID NO: 3 - protein, 9 aa, Artificial Sequence
  Chemically Synthesized, CD9B366 HCDR3
DPILAALDY

SEQ ID NO: 4 - protein, 16 aa, Artificial Sequence
  Chemically Synthesized, CD9B366 LCDR1
RSSQSLVYSDGNTYLH

SEQ ID NO: 5 - protein, 7 aa, Artificial Sequence
  Chemically Synthesized, CD9B366 LCDR2
KVSNRDS

SEQ ID NO: 6 - protein, 9 aa, Artificial Sequence
  Chemically Synthesized, CD9B366 LCDR3
MQGTHWPRT

SEQ ID NO: 7 - protein, 118 aa, Artificial Sequence
  Chemically Synthesized, CD9B366 VH
QVQLVESGGGLVQPGGSLRLSCSASGFTFSNYWMSWVRQAPGKGLEWVANINQGGSEKYYVDSVEGRFTISRDNAKNSLSLQMNGLRAEDTAVYYCARDPILAALDYWGQGTLVTVSS

SEQ ID NO: 8 - protein, 111 aa, Artificial Sequence
  Chemically Synthesized, CD9B366 VL
DIVMTQSPLSLSVTLGQPASISCRSSQSLVYSDGNTYLHWFHQRPGQSPRRLIYKVSNRDSGVPDRFSGTGSDTDFTLKISRVEAEDVGVYYCMQGTHWPRTFGQGTKVEI

SEQ ID NO: 9 - protein, 448 aa, Artificial Sequence
  Chemically Synthesized, CD9B366 HC
QVQLVESGGGLVQPGGSLRLSCSASGFTFSNYWMSWVRQAPGKGLEWVANINQGGSEKYYVDSVEGRFTISRDNAKNSLSLQMNGLRAEDTAVYYCARDPILAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  Chemically Synthesized, CD9B366 LC
DIVMTQSPLSLSVTLGQPASISCRSSQSLVYSDGNTYLHWFHQRPGQSPRRLIYKVSNRDSGVPDRFSGTGSDTDFTLKISRVEAEDVGVYYCMQGTHWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, Artificial Sequence
  Chemically Synthesized, CD9B366m1 HCDR1
GFTFSNYWMS

SEQ ID NO: 12 - protein, 10 aa, Artificial Sequence
  Chemically Synthesized, CD9B366m1 HCDR2
NINQGGSEKY

SEQ ID NO: 13 - protein, 9 aa, Artificial Sequence
  Chemically Synthesized, CD9B366m1 HCDR3
EPILAALDY

SEQ ID NO: 14 - protein, 16 aa, Artificial Sequence
  Chemically Synthesized, CD9B366m1 LCDR1
RSSQSLVYSDGNTYLH

SEQ ID NO: 15 - protein, 7 aa, Artificial Sequence
  Chemically Synthesized, CD9B366m1 LCDR2
KVSNRDS

SEQ ID NO: 16 - protein, 9 aa, Artificial Sequence
  Chemically Synthesized, CD9B366m1 LCDR3
MQGTHWPRT

SEQ ID NO: 17 - protein, 118 aa, Artificial Sequence
  Chemically Synthesized, CD9B366m1 VH
QVQLVESGGGLVQPGGSLRLSCSASGFTFSNYWMSWVRQAPGKGLEWVANINQGGSEKYYVDSVEGRFTISRDNAKNSLSLQMNGLRAEDTAVYYCAREPILAALDYWGQGTLVTVSS

SEQ ID NO: 18 - protein, 111 aa, Artificial Sequence; identical to SEQ ID NO: 8
  Chemically Synthesized, CD9B366m1 VL

SEQ ID NO: 19 - protein, 448 aa, Artificial Sequence
  Chemically Synthesized, CD9B366m1 HC
QVQLVESGGGLVQPGGSLRLSCSASGFTFSNYWMSWVRQAPGKGLEWVANINQGGSEKYYVDSVEGRFTISRDNAKNSLSLQMNGLRAEDTAVYYCAREPILAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, Artificial Sequence; identical to SEQ ID NO: 10
  Chemically Synthesized, CD9B366m1 LC

SEQ ID NO: 21 - protein, 10 aa, Artificial Sequence
  Chemically Synthesized, CD9B376 HCDR1
GFTFNNYWMS

SEQ ID NO: 22 - protein, 10 aa, Artificial Sequence
  Chemically Synthesized, CD9B376 HCDR2
NIYRDGSEKY

SEQ ID NO: 23 - protein, 9 aa, Artificial Sequence
  Chemically Synthesized, CD9B376 HCDR3
DPSTGDLDY

SEQ ID NO: 24 - protein, 16 aa, Artificial Sequence
  Chemically Synthesized, CD9B376 LCDR1
RSSQSLVYHDGNTYLI

SEQ ID NO: 25 - protein, 7 aa, Artificial Sequence
  Chemically Synthesized, CD9B376 LCDR2
KVSNRDS

SEQ ID NO: 26 - protein, 9 aa, Artificial Sequence
  Chemically Synthesized, CD9B376 LCDR3
MQGSHWPRT

SEQ ID NO: 27 - protein, 118 aa, Artificial Sequence
  Chemically Synthesized, CD9B376 VH
EVQLVESGGDLVQPGGSLRLSCIASGFTFNNYWMSWVRQAPGKGLEWVANIYRDGSEKYYLDSVEGRFTISRDNAKNSLYLQMNSLRLEDTAVYYCARDPSTGDLDYWGQGTPVTVSS

SEQ ID NO: 28 - protein, 112 aa, Artificial Sequence
  Chemically Synthesized, CD9B376 VL
DIQMTQSPLSLSVTLGQPASISCRSSQSLVYHDGNTYLIWFQQRPGQSPRRLIYKVSNRDSGVPDRFSGSGSGTDFTLKISRVEAEDVGVYYCMQGSHWPRTFGPGTKVDIK

SEQ ID NO: 29 - protein, 448 aa, Artificial Sequence
  Chemically Synthesized, CD9B376 HC
EVQLVESGGDLVQPGGSLRLSCIASGFTFNNYWMSWVRQAPGKGLEWVANIYRDGSEKYYLDSVEGRFTISRDNAKNSLYLQMNSLRLEDTAVYYCARDPSTGDLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 219 aa, Artificial Sequence
  Chemically Synthesized, CD9B376 LC
DIQMTQSPLSLSVTLGQPASISCRSSQSLVYHDGNTYLIWFQQRPGQSPRRLIYKVSNRDSGVPDRFSGSGSGTDFTLKISRVEAEDVGVYYCMQGSHWPRTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 10 aa, Artificial Sequence
  Chemically Synthesized, CD9B376m1 HCDR1
GFTFNNYWMS

SEQ ID NO: 32 - protein, 10 aa, Artificial Sequence
  Chemically Synthesized, CD9B376m1 HCDR2
NIYRDGSEKY

SEQ ID NO: 33 - protein, 9 aa, Artificial Sequence
  Chemically Synthesized, CD9B376m1 HCDR3
EPSTGDLDY

SEQ ID NO: 34 - protein, 16 aa, Artificial Sequence
  Chemically Synthesized, CD9B376m1 LCDR1
RSSQSLVYHDGNTYLI

SEQ ID NO: 35 - protein, 7 aa, Artificial Sequence
  Chemically Synthesized, CD9B376m1 LCDR2
KVSNRDS

SEQ ID NO: 36 - protein, 9 aa, Artificial Sequence
  Chemically Synthesized, CD9B376m1 LCDR3
MQGSHWPRT

SEQ ID NO: 37 - protein, 118 aa, Artificial Sequence
  Chemically Synthesized, CD9B376m1 VH
EVQLVESGGDLVQPGGSLRLSCIASGFTFNNYWMSWVRQAPGKGLEWVANIYRDGSEKYYLDSVEGRFTISRDNAKNSLYLQMNSLRLEDTAVYYCAREPSTGDLDYWGQGTPVTVSS

SEQ ID NO: 38 - protein, 112 aa, Artificial Sequence; identical to SEQ ID NO: 28
  Chemically Synthesized, CD9B376m1 VL

SEQ ID NO: 39 - protein, 448 aa, Artificial Sequence
  Chemically Synthesized, CD9B376m1 HC
EVQLVESGGDLVQPGGSLRLSCIASGFTFNNYWMSWVRQAPGKGLEWVANIYRDGSEKYYLDSVEGRFTISRDNAKNSLYLQMNSLRLEDTAVYYCAREPSTGDLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 219 aa, Artificial Sequence; identical to SEQ ID NO: 30
  Chemically Synthesized, CD9B376m1 LC

SEQ ID NO: 41 - protein, 10 aa, Artificial Sequence
  Chemically Synthesized, CD9B378 HCDR1
GFTFGTYAMN

SEQ ID NO: 42 - protein, 10 aa, Artificial Sequence
  Chemically Synthesized, CD9B378 HCDR2
YITTSSSHIY

SEQ ID NO: 43 - protein, 15 aa, Artificial Sequence
  Chemically Synthesized, CD9B378 HCDR3
DYGSSSSEDYYGMDV

SEQ ID NO: 44 - protein, 17 aa, Artificial Sequence
  Chemically Synthesized, CD9B378 LCDR1
RSGQSLLDSDDGNIYLD

SEQ ID NO: 45 - protein, 7 aa, Artificial Sequence
  Chemically Synthesized, CD9B378 LCDR2
TVSYRAS

SEQ ID NO: 46 - protein, 9 aa, Artificial Sequence
  Chemically Synthesized, CD9B378 LCDR3
MQRIDFPFT

SEQ ID NO: 47 - protein, 124 aa, Artificial Sequence
  Chemically Synthesized, CD9B378 VH
EVQLVQSGGGLVKPGGSLRLSCAASGFTFGTYAMNWVRQAPGKGLEWVSYITTSSSHIYYSDSVKGRFTISRDNAKNSLYLQMNSLRAEDTAVYYCTRDYGSSSSEDYYGMDVWGQGTMVTVSS

SEQ ID NO: 48 - protein, 113 aa, Artificial Sequence
  Chemically Synthesized, CD9B378 VL
EIVMTQSPLYLPVTPGEPASISCRSGQSLLDSDDGNIYLDWFLQKPGQSPQLLIYTVSYRASGVPDRFSGSGSGTDFTLKISRVEAEDVGVYYCMQRIDFPFTFGPGTKVDIK

SEQ ID NO: 49 - protein, 454 aa, Artificial Sequence
  Chemically Synthesized, CD9B378 HC
EVQLVQSGGGLVKPGGSLRLSCAASGFTFGTYAMNWVRQAPGKGLEWVSYITTSSSHIYYSDSVKGRFTISRDNAKNSLYLQMNSLRAEDTAVYYCTRDYGSSSSEDYYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 220 aa, Artificial Sequence
  Chemically Synthesized, CD9B378 LC
EIVMTQSPLYLPVTPGEPASISCRSGQSLLDSDDGNIYLDWFLQKPGQSPQLLIYTVSYRASGVPDRFSGSGSGTDFTLKISRVEAEDVGVYYCMQRIDFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 10 aa, Artificial Sequence
  Chemically Synthesized, CD9B164 HCDR1
GFIFSTYWMS

SEQ ID NO: 52 - protein, 10 aa, Artificial Sequence
  Chemically Synthesized, CD9B164 HCDR2
NINPDGNEKY

SEQ ID NO: 53 - protein, 9 aa, Artificial Sequence
  Chemically Synthesized, CD9B164 HCDR3
DPISARFDF

SEQ ID NO: 54 - protein, 16 aa, Artificial Sequence
  Chemically Synthesized, CD9B164 LCDR1
RSSQSLEYSDGNTYLN

SEQ ID NO: 55 - protein, 7 aa, Artificial Sequence
  Chemically Synthesized, CD9B164 LCDR2
KVSNRDS

SEQ ID NO: 56 - protein, 9 aa, Artificial Sequence
  Chemically Synthesized, CD9B164 LCDR3
MQGTHWPPT

SEQ ID NO: 57 - protein, 118 aa, Artificial Sequence
  Chemically Synthesized, CD9B164 VH
EVQLVESGGGLVQPGGSLRLSCAASGFIFSTYWMSWVRQAPGKGLEWVANINPDGNEKYYVDSVEGRFTISRDNAQDSLYLQMNSLRAEDTAVYYCARDPISARFDFWGQGTLVTVSS

SEQ ID NO: 58 - protein, 112 aa, Artificial Sequence
  Chemically Synthesized, CD9B164 VL
EIVMTQSPLSLPVTPGKPASISCRSSQSLEYSDGNTYLNWFQQRPGQSPRRLIYKVSNRDSGVPDRFSGSGSGTDFTLKISRVEAEDVGVYYCMQGTHWPPTFGGGTKVDIK

SEQ ID NO: 59 - protein, 448 aa, Artificial Sequence
  Chemically Synthesized, CD9B164 HC
EVQLVESGGGLVQPGGSLRLSCAASGFIFSTYWMSWVRQAPGKGLEWVANINPDGNEKYYVDSVEGRFTISRDNAQDSLYLQMNSLRAEDTAVYYCARDPISARFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, Artificial Sequence
  Chemically Synthesized, CD9B164 LC
EIVMTQSPLSLPVTPGKPASISCRSSQSLEYSDGNTYLNWFQQRPGQSPRRLIYKVSNRDSGVPDRFSGSGSGTDFTLKISRVEAEDVGVYYCMQGTHWPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 10 aa, Artificial Sequence
  Chemically Synthesized, CD9B164m1 HCDR1
GFIFSTYWMS

SEQ ID NO: 62 - protein, 10 aa, Artificial Sequence
  Chemically Synthesized, CD9B164m1 HCDR2
NINPDGNEKY

SEQ ID NO: 63 - protein, 9 aa, Artificial Sequence
  Chemically Synthesized, CD9B164m1 HCDR3
EPISARFDF

SEQ ID NO: 64 - protein, 16 aa, Artificial Sequence
  Chemically Synthesized, CD9B164m1 LCDR1
RSSQSLEYSDGNTYLN

SEQ ID NO: 65 - protein, 7 aa, Artificial Sequence
  Chemically Synthesized, CD9B164m1 LCDR2
KVSNRDS

SEQ ID NO: 66 - protein, 9 aa, Artificial Sequence
  Chemically Synthesized, CD9B164m1 LCDR3
MQGTHWPPT

SEQ ID NO: 67 - protein, 118 aa, Artificial Sequence
  Chemically Synthesized, CD9B164m1 VH
EVQLVESGGGLVQPGGSLRLSCAASGFIFSTYWMSWVRQAPGKGLEWVANINPDGNEKYYVDSVEGRFTISRDNAQDSLYLQMNSLRAEDTAVYYCAREPISARFDFWGQGTLVTVSS

SEQ ID NO: 68 - protein, 111 aa, Artificial Sequence
  Chemically Synthesized, CD9B164m1 VL
EIVMTQSPLSLPVTPGKPASISCRSSQSLEYSDGNTYLNWFQQRPGQSPRRLIYKVSNRDSGVPDRFSGSGSGTDFTLKISRVEAEDVGVYYCMQGTHWPPTFGGGTKVDI

SEQ ID NO: 69 - protein, 448 aa, Artificial Sequence
  Chemically Synthesized, CD9B164m1 HC
EVQLVESGGGLVQPGGSLRLSCAASGFIFSTYWMSWVRQAPGKGLEWVANINPDGNEKYYVDSVEGRFTISRDNAQDSLYLQMNSLRAEDTAVYYCAREPISARFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 219 aa, Artificial Sequence; identical to SEQ ID NO: 60
  Chemically Synthesized, CD9B164m1 LC

SEQ ID NO: 71 - protein, 10 aa, Artificial Sequence
  Chemically Synthesized, CD9B312 HCDR1
GFTFSRHTMN

SEQ ID NO: 72 - protein, 10 aa, Artificial Sequence
  Chemically Synthesized, CD9B312 HCDR2
SISGSGGSTY

SEQ ID NO: 73 - protein, 13 aa, Artificial Sequence
  Chemically Synthesized, CD9B312 HCDR3
ESYDSSGVWYFDL

SEQ ID NO: 74 - protein, 11 aa, Artificial Sequence
  Chemically Synthesized, CD9B312 LCDR1
SGHKLGDKYAC

SEQ ID NO: 75 - protein, 7 aa, Artificial Sequence
  Chemically Synthesized,  CD9B312 LCDR2
QDSKRPS

SEQ ID NO: 76 - protein, 10 aa, Artificial Sequence
  Chemically Synthesized,  CD9B312 LCDR3
QAWDSITAWV

SEQ ID NO: 77 - protein, 122 aa, Artificial Sequence
  Chemically Synthesized,  CD9B312 VH
EVQLVESGGGLVQSGGSLRISCVASGFTFSRHTMNWVRQAPGKGLEWVSSISGSGGSTYYADSVKGRLTVSRDNSKNTLYLQMNSLRAEDTAVYYCAKESYDSSGVWYFDLWGRGTLVTVSS

SEQ ID NO: 78 - protein, 107 aa, Artificial Sequence
  Chemically Synthesized, CD9B312 VL
SSELTQPPSMSVSPGQTANIACSGHKLGDKYACWYQQKPGQSPVLVIYQDSKRPSGIPERFSGSNSGNTATLTISGTQAMDEADYYCQAWDSITAWVFGGGTKVTVL

SEQ ID NO: 79 - protein, 452 aa, Artificial Sequence
  Chemically Synthesized, CD9B312 HC
EVQLVESGGGLVQSGGSLRISCVASGFTFSRHTMNWVRQAPGKGLEWVSSISGSGGSTYYADSVKGRLTVSRDNSKNTLYLQMNSLRAEDTAVYYCAKESYDSSGVWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 213 aa, Artificial Sequence
  Chemically Synthesized, CD9B312 LC
SSELTQPPSMSVSPGQTANIACSGHKLGDKYACWYQQKPGQSPVLVIYQDSKRPSGIPERFSGSNSGNTATLTISGTQAMDEADYYCQAWDSITAWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - protein, 10 aa, Artificial Sequence
  Chemically Synthesized, CD9B312m1 HCDR1
GFTFSRHTMN

SEQ ID NO: 82 - protein, 10 aa, Artificial Sequence
  Chemically Synthesized, CD9B312m1 HCDR2
SISGSGGSTY

SEQ ID NO: 83 - protein, 13 aa, Artificial Sequence
  Chemically Synthesized, CD9B312m1 HCDR3
ESYDSSGVWYFDL

SEQ ID NO: 84 - protein, 11 aa, Artificial Sequence
  Chemically Synthesized, CD9B312m1 LCDR1
SGHKLGDKYAS

SEQ ID NO: 85 - protein, 7 aa, Artificial Sequence
  Chemically Synthesized, CD9B312m1 LCDR2
QDSKRPS

SEQ ID NO: 86 - protein, 10 aa, Artificial Sequence
  Chemically Synthesized, CD9B312m1 LCDR3
QAWDSITAWV

SEQ ID NO: 87 - protein, 122 aa, Artificial Sequence; identical to SEQ ID NO: 77
  Chemically Synthesized, CD9B312m1 VH

SEQ ID NO: 88 - protein, 107 aa, Artificial Sequence
  Chemically Synthesized, CD9B312m1 VL
SSELTQPPSMSVSPGQTANIACSGHKLGDKYASWYQQKPGQSPVLVIYQDSKRPSGIPERFSGSNSGNTATLTISGTQAMDEADYYCQAWDSITAWVFGGGTKVTVL

SEQ ID NO: 89 - protein, 452 aa, Artificial Sequence; identical to SEQ ID NO: 79
  Chemically Synthesized, CD9B312m1 HC

SEQ ID NO: 90 - protein, 213 aa, Artificial Sequence
  Chemically Synthesized, CD9B312m1 LC
SSELTQPPSMSVSPGQTANIACSGHKLGDKYASWYQQKPGQSPVLVIYQDSKRPSGIPERFSGSNSGNTATLTISGTQAMDEADYYCQAWDSITAWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 91 - protein, 10 aa, Artificial Sequence
  Chemically Synthesized, CD9B292 HCDR1
GFTFNSYNMN

SEQ ID NO: 92 - protein, 10 aa, Artificial Sequence
  Chemically Synthesized, CD9B292 HCDR2
YISSSGYTIY

SEQ ID NO: 93 - protein, 18 aa, Artificial Sequence
  Chemically Synthesized, CD9B292 HCDR
DRYNYDNGGYHYYTGMDV

SEQ ID NO: 94 - protein, 11 aa, Artificial Sequence
  Chemically Synthesized, CD9B292 LCDR1
SGDKLGDKFAC

SEQ ID NO: 95 - protein, 7 aa, Artificial Sequence
  Chemically Synthesized, CD9B292 LCDR2
QDSKRPS

SEQ ID NO: 96 - protein, 10 aa, Artificial Sequence
  Chemically Synthesized, CD9B292 LCDR3
QAWDSTTAWV

SEQ ID NO: 97 - protein, 127 aa, Artificial Sequence
  Chemically Synthesized, CD9B292 VH
EVQLVESGGGLVQPGGSLRLSCAASGFTFNSYNMNWVRQAPGKGLEWVSYISSSGYTIYYADSVEGRFTISRDNAKNSLYLQMDSLRDEDTAIYYCARDRYNYDNGGYHYYTGMDVWGQGTMVTVSS

SEQ ID NO: 98 - protein, 107 aa, Artificial Sequence
  Chemically Synthesized, CD9B292 VL
SSELTQPPSVSVSPGQTASITCSGDKLGDKFACWYQQKPGQSPVLVIFQDSKRPSGIPERFSGSNFGNTATLTISGTQAMDEADYFCQAWDSTTAWVFGGGTKVTVL

SEQ ID NO: 99 - protein, 457 aa, Artificial Sequence
  Chemically Synthesized, CD9B292 HC
EVQLVESGGGLVQPGGSLRLSCAASGFTFNSYNMNWVRQAPGKGLEWVSYISSSGYTIYYADSVEGRFTISRDNAKNSLYLQMDSLRDEDTAIYYCARDRYNYDNGGYHYYTGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 213 aa, Artificial Sequence
  Chemically Synthesized, CD9B292 LC
SSELTQPPSVSVSPGQTASITCSGDKLGDKFACWYQQKPGQSPVLVIFQDSKRPSGIPERFSGSNFGNTATLTISGTQAMDEADYFCQAWDSTTAWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 101 - protein, 10 aa, Artificial Sequence
  Chemically Synthesized, CD9B292m1 HCDR1
GFTFNSYNMN

SEQ ID NO: 102 - protein, 10 aa, Artificial Sequence
  Chemically Synthesized, CD9B292m1 HCDR2
YISSSGYTIY

SEQ ID NO: 103 - protein, 18 aa, Artificial Sequence
  Chemically Synthesized, CD9B292m1 HCDR3
DRYNYDNGGYHYYTGMDV

SEQ ID NO: 104 - protein, 11 aa, Artificial Sequence
  Chemically Synthesized, CD9B292m1 LCDR1
SGDKLGDKFAS

SEQ ID NO: 105 - protein, 7 aa, Artificial Sequence
  Chemically Synthesized, CD9B292m1 LCDR2
QDSKRPS

SEQ ID NO: 106 - protein, 10 aa, Artificial Sequence
  Chemically Synthesized, CD9B292m1 LCDR3
QAWDSTTAWV

SEQ ID NO: 107 - protein, 127 aa, Artificial Sequence; identical to SEQ ID NO: 97
  Chemically Synthesized, CD9B292m1 VH

SEQ ID NO: 108 - protein, 107 aa, Artificial Sequence
  Chemically Synthesized, CD9B292m1 VL
SSELTQPPSVSVSPGQTASITCSGDKLGDKFASWYQQKPGQSPVLVIFQDSKRPSGIPERFSGSNFGNTATLTISGTQAMDEADYFCQAWDSTTAWVFGGGTKVTVL

SEQ ID NO: 109 - protein, 457 aa, Artificial Sequence; identical to SEQ ID NO: 99
  Chemically Synthesized, CD9B292m1 HC

SEQ ID NO: 110 - protein, 213 aa, Artificial Sequence
  Chemically Synthesized, CD9B292m1 LC
SSELTQPPSVSVSPGQTASITCSGDKLGDKFASWYQQKPGQSPVLVIFQDSKRPSGIPERFSGSNFGNTATLTISGTQAMDEADYFCQAWDSTTAWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 5 aa, Artificial Sequence
  Chemically Synthesized, CD9B1256 HCDR1
SYYWS

SEQ ID NO: 112 - protein, 16 aa, Artificial Sequence
  Chemically Synthesized, CD9B1256 HCDR2
RIYSSGSTNYNPSLKS

SEQ ID NO: 113 - protein, 14 aa, Artificial Sequence
  Chemically Synthesized, CD9B1256 HCDR3
GEYSGDLGGYGLDV

SEQ ID NO: 114 - protein, 17 aa, Artificial Sequence
  Chemically Synthesized, CD9B1256 LCDR1
RSSQSLLDSDDGNTYLD

SEQ ID NO: 115 - protein, 7 aa, Artificial Sequence
  Chemically Synthesized, CD9B1256 LCDR2
TLSYRAS

SEQ ID NO: 116 - protein, 9 aa, Artificial Sequence
  Chemically Synthesized, CD9B1256 LCDR3
MQRMEFPLT

SEQ ID NO: 117 - protein, 122 aa, Artificial Sequence
  Chemically Synthesized, CD9B1256 VH
EVQLVESGPGLVEPSETLSLTCTVSGGSISSYYWSWIRQPAGKGLEWIGRIYSSGSTNYNPSLKSRVTMSVDTSENQFSLNLNSVTAADTAKYYCARGEYSGDLGGYGLDVWGQGTMVTVSS

SEQ ID NO: 118 - protein, 113 aa, Artificial Sequence
  Chemically Synthesized, CD9B1256 VL
EIVMTQSPLSLSVTPGEPASISCRSSQSLLDSDDGNTYLDWYLQKPGQSPQLLIQTLSYRASGVPDRFSGRGSGTDFTLKISRVEAEDVGIYYCMQRMEFPLTFGGGTKVDIK

SEQ ID NO: 119 - protein, 452 aa, Artificial Sequence
  Chemically Synthesized, CD9B1256 HC
EVQLVESGPGLVEPSETLSLTCTVSGGSISSYYWSWIRQPAGKGLEWIGRIYSSGSTNYNPSLKSRVTMSVDTSENQFSLNLNSVTAADTAKYYCARGEYSGDLGGYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 220 aa, Artificial Sequence
  Chemically Synthesized, CD9B1256 LC
EIVMTQSPLSLSVTPGEPASISCRSSQSLLDSDDGNTYLDWYLQKPGQSPQLLIQTLSYRASGVPDRFSGRGSGTDFTLKISRVEAEDVGIYYCMQRMEFPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 5 aa, Artificial Sequence
  Chemically Synthesized, CD9B1218 HCDR1
SYYWS

SEQ ID NO: 122 - protein, 16 aa, Artificial Sequence
  Chemically Synthesized, CD9B1218 HCDR2
RIHTSGNTNYNPSLKS

SEQ ID NO: 123 - protein, 14 aa, Artificial Sequence
  Chemically Synthesized, CD9B1218 HCDR3
GEYSGDLGGYGLDV

SEQ ID NO: 124 - protein, 17 aa, Artificial Sequence
  Chemically Synthesized, CD9B1218 LCDR1
RSSQSLLDRDDGNTYLD

SEQ ID NO: 125 - protein, 7 aa, Artificial Sequence
  Chemically Synthesized, CD9B1218 LCDR2
TLSYRAS

SEQ ID NO: 126 - protein, 9 aa, Artificial Sequence
  Chemically Synthesized, CD9B1218 LCDR3
MQRIEFPLT

SEQ ID NO: 127 - protein, 122 aa, Artificial Sequence
  Chemically Synthesized, CD9B1218 VH
QVQLQESGPGLVKPSETLSLTCTVSGVSISSYYWSWIRQPAGKGLEWIGRIHTSGNTNYNPSLKSRATMSGDTSKNHFSLKLTSVTAADTAVYYCARGEYSGDLGGYGLDVWGQGTMVTVSS

SEQ ID NO: 128 - protein, 113 aa, Artificial Sequence
  Chemically Synthesized, CD9B1218 VL
DIVMTQTPLSLPVTPGEPASISCRSSQSLLDRDDGNTYLDWYLQKPGQSPQLLIYTLSYRASGVPDRFSGSGSGTDFTLKISRVEAEDVGVYYCMQRIEFPLTFGGGTKVEIK

SEQ ID NO: 129 - protein, 452 aa, Artificial Sequence
  Chemically Synthesized, CD9B1218 HC
QVQLQESGPGLVKPSETLSLTCTVSGVSISSYYWSWIRQPAGKGLEWIGRIHTSGNTNYNPSLKSRATMSGDTSKNHFSLKLTSVTAADTAVYYCARGEYSGDLGGYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 220 aa, Artificial Sequence
  Chemically Synthesized, CD9B1218 LC
DIVMTQTPLSLPVTPGEPASISCRSSQSLLDRDDGNTYLDWYLQKPGQSPQLLIYTLSYRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 5 aa, Artificial Sequence
  Chemically Synthesized, CD9B1315 HCDR1
SYYWS

SEQ ID NO: 132 - protein, 16 aa, Artificial Sequence
  Chemically Synthesized, CD9B1315 HCDR2
RIYTRGSTHYNPSLKS

SEQ ID NO: 133 - protein, 14 aa, Artificial Sequence
  Chemically Synthesized, CD9B1315 HCDR3
GEYSGDLGGYGLDV

SEQ ID NO: 134 - protein, 17 aa, Artificial Sequence
  Chemically Synthesized, CD9B1315 LCDR1
RSSQSLLDRDDGKTYLD

SEQ ID NO: 135 - protein, 7 aa, Artificial Sequence
  Chemically Synthesized, CD9B1315 LCDR2
TLSDRAS

SEQ ID NO: 136 - protein, 9 aa, Artificial Sequence
  Chemically Synthesized, CD9B1315 LCDR3
MQRIEFPLT

SEQ ID NO: 137 - protein, 122 aa, Artificial Sequence
  Chemically Synthesized, CD9B1315 VH
EVQLVESGPELVKPSETLSLTCTVSGVSISSYYWSWIRQPAGKGLEWIGRIYTRGSTHYNPSLKSRVTMSEDTSKNQFFLKLGSVTAADTAVYYCARGEYSGDLGGYGLDVWGQGTMVTVSS

SEQ ID NO: 138 - protein, 113 aa, Artificial Sequence
  Chemically Synthesized, CD9B1315 VL
DIVMTQSPLSLPVTPGEPASISCRSSQSLLDRDDGKTYLDWYLQKPGQSPQLLIYTLSDRASGVPDRFSGSGSGTDFTLKISRVEADDVGVYYCMQRIEFPLTFGGGTKLEIK

SEQ ID NO: 139 - protein, 452 aa, Artificial Sequence
  Chemically Synthesized, CD9B1315 HC
EVQLVESGPELVKPSETLSLTCTVSGVSISSYYWSWIRQPAGKGLEWIGRIYTRGSTHYNPSLKSRVTMSEDTSKNQFFLKLGSVTAADTAVYYCARGEYSGDLGGYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 220 aa, Artificial Sequence
  Chemically Synthesized, CD9B1315 LC
DIVMTQSPLSLPVTPGEPASISCRSSQSLLDRDDGKTYLDWYLQKPGQSPQLLIYTLSDRASGVPDRFSGSGSGTDFTLKISRVEADDVGVYYCMQRIEFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 20 aa, Artificial Sequence
  Chemically Synthesized, linker
GGSEGKSSGSGSESKSTGGS

SEQ ID NO: 142 - protein, 8 aa, Artificial Sequence
  Chemically Synthesized, linker
GGGSGGGS

SEQ ID NO: 143 - protein, 12 aa, Artificial Sequence
  Chemically Synthesized, linker
GGGSGGGSGGGS

SEQ ID NO: 144 - protein, 16 aa, Artificial Sequence
  Chemically Synthesized, linker
GGGSGGGSGGGSGGGS

SEQ ID NO: 145 - protein, 20 aa, Artificial Sequence
  Chemically Synthesized, linker
GGGSGGGSGGGSGGGSGGGS

SEQ ID NO: 146 - protein, 15 aa, Artificial Sequence
  Chemically Synthesized, linker
GGGGSGGGGSGGGGS

SEQ ID NO: 147 - protein, 20 aa, Artificial Sequence
  Chemically Synthesized, linker
GGGGSGGGGSGGGGSGGGGS

SEQ ID NO: 148 - protein, 25 aa, Artificial Sequence
  Chemically Synthesized, linker
GGGGSGGGGSGGGGSGGGGSGGGGS

SEQ ID NO: 149 - protein, 18 aa, Artificial Sequence
  Chemically Synthesized, linker
GSTSGSGKPGSGEGSTKG

SEQ ID NO: 150 - protein, 14 aa, Artificial Sequence
  Chemically Synthesized, linker
IRPRAIGGSKPRVA

SEQ ID NO: 151 - protein, 15 aa, Artificial Sequence
  Chemically Synthesized, linker
GKGGSGKGGSGKGGS

SEQ ID NO: 152 - protein, 15 aa, Artificial Sequence
  Chemically Synthesized, linker
GGKGSGGKGSGGKGS

SEQ ID NO: 153 - protein, 15 aa, Artificial Sequence
  Chemically Synthesized, linker
GGGKSGGGKSGGGKS

SEQ ID NO: 154 - protein, 15 aa, Artificial Sequence
  Chemically Synthesized, linker
GKGKSGKGKSGKGKS

SEQ ID NO: 155 - protein, 15 aa, Artificial Sequence
  Chemically Synthesized, linker
GGGKSGGKGSGKGGS

SEQ ID NO: 156 - protein, 15 aa, Artificial Sequence
  Chemically Synthesized, linker
GKPGSGKPGSGKPGS

SEQ ID NO: 157 - protein, 20 aa, Artificial Sequence
  Chemically Synthesized, linker
GKPGSGKPGSGKPGSGKPGS

SEQ ID NO: 158 - protein, 20 aa, Artificial Sequence
  Chemically Synthesized, linker
GKGKSGKGKSGKGKSGKGKS

SEQ ID NO: 159 - protein, 14 aa, Artificial Sequence
  Chemically Synthesized, linker
STAGDTHLGGEDFD

SEQ ID NO: 160 - protein, 15 aa, Artificial Sequence
  Chemically Synthesized, linker
GEGGSGEGGSGEGGS

SEQ ID NO: 161 - protein, 15 aa, Artificial Sequence
  Chemically Synthesized, linker
GGEGSGGEGSGGEGS

SEQ ID NO: 162 - protein, 15 aa, Artificial Sequence
  Chemically Synthesized, linker
GEGESGEGESGEGES

SEQ ID NO: 163 - protein, 15 aa, Artificial Sequence
  Chemically Synthesized, linker
GGGESGGEGSGEGGS

SEQ ID NO: 164 - protein, 20 aa, Artificial Sequence
  Chemically Synthesized, linker
GEGESGEGESGEGESGEGES

SEQ ID NO: 165 - protein, 18 aa, Artificial Sequence
  Chemically Synthesized, linker
GSTSGSGKPGSGEGSTKG

SEQ ID NO: 166 - protein, 19 aa, Artificial Sequence
  Chemically Synthesized, linker
PRGASKSGSASQTGSAPGS

SEQ ID NO: 167 - protein, 19 aa, Artificial Sequence
  Chemically Synthesized, linker
GTAAAGAGAAGGAAAGAAG

SEQ ID NO: 168 - protein, 19 aa, Artificial Sequence
  Chemically Synthesized, linker
GTSGSSGSGSGGSGSGGGG

SEQ ID NO: 169 - protein, 20 aa, Artificial Sequence
  Chemically Synthesized, linker
GKPGSGKPGSGKPGSGKPGS

SEQ ID NO: 170 - protein, 4 aa, Artificial Sequence
  Chemically Synthesized, linker
GSGS

SEQ ID NO: 171 - protein, 10 aa, Artificial Sequence
  Chemically Synthesized, linker
APAPAPAPAP

SEQ ID NO: 172 - protein, 20 aa, Artificial Sequence
  Chemically Synthesized, linker
APAPAPAPAPAPAPAPAPAP

SEQ ID NO: 173 - protein, 32 aa, Artificial Sequence
  Chemically Synthesized, linker
AEAAAKEAAAKEAAAAKEAAAAKEAAAAKAAA

SEQ ID NO: 174 - protein, 330 aa, Artificial Sequence [sequence omitted]

SEQ ID NO: 175 - protein, 326 aa, Artificial Sequence
  Chemically Synthesized, wild-type IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 176 - protein, 327 aa, Artificial Sequence
  Chemically Synthesized, wild-type IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 177 - protein, 229 aa, Artificial Sequence
  Chemically Synthesized, full length CD79b
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 178 - protein, 176 aa, Artificial Sequence
  Chemically Synthesized, CD79b long isoform
MARSALLILALLLLGLFSPGAWGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGSLNDIFEAQKIEWHEHHHHHH

SEQ ID NO: 179 - protein, 72 aa, Artificial Sequence
  Chemically Synthesized, CD79b short isoform
MARSALLILALLLLGLFSPGAWGARSEDRYRNPKGFSTLAQLKQRNTLKDGSLNDIFEAQKIEWHEHHHHHH

(19) United States
(12) Patent Application Publication
(10) Pub. No.: US 2024/0158499 A1
(43) Pub. Date: May 16, 2024

(54) USES OF CD79B ANTIBODIES FOR AUTOIMMUNE THERAPEUTIC APPLICATIONS

(71) Applicant: JANSSEN BIOTECH, INC., Horsham, PA (US)

(72) Inventors: Songmao Zheng, Ambler, PA (US); John Emery, Spring House, PA (US); Jennifer F. Nemeth, Spring House, PA (US); Fang Shen, Fort Washington, PA (US); Pankaj Seth, Spring House, PA (US)

(21) Appl. No.: 18/549,665

(22) PCT Filed: Mar. 11, 2022
(86) PCT No.: PCT/US22/19914

§ 371 Date: Sep. 8, 2023

Related U.S. Application Data
(60) Provisional application No. 63/252,910, filed on Oct. 6, 2021.
(60) Provisional application No. 63/160,127, filed on Mar. 12, 2021.

(51) Int. Cl. C07K 16/28; A61K 39/00

(52) U.S. Cl. CPC C07K 16/2803; A61K 2039/505; C07K 2317/565; C07K 2317/622

(57) ABSTRACT

The invention provides antigen binding domains that bind Cluster of Differentiation 79B protein (CD79B) protein comprising the antigen binding domains that bind CD79b, polynucleotides encoding them, vectors, host cells, methods of making and using them.

### CROSS REFERENCE TO RELATED APPLICATIONS

This application claims priority to U.S. Provisional Application No. 63/160,127, filed Mar. 12, 2021 and to U.S. Provisional Application No. 63/252,910, filed Oct. 6, 2021, each of which is hereby incorporated by reference herein in its entirety.

### TECHNICAL FIELD

The invention provides antigen binding domains that bind Cluster of Differentiation 79B protein (CD79B) protein comprising the antigen binding domains that bind CD79b, polynucleotides encoding them, vectors, host cells, methods of making and using them.

### BACKGROUND

The prevalence of autoimmune disease is estimated to be 3-5% of the general population, and dysregulation of B cells, autoreactive B cells, and the presence of autoantibodies is a common feature of many autoimmune diseases (Wang et al, 2015, J Intern Med 2015; 278: 369-395).

B cells, or B lymphocytes, are central components of adaptive immunity, responding to different pathogens by producing antibodies, performing the role of antigen-presenting cells, secreting cytokines, and developing into memory B cells after activation (Packard and Cambier, 2013, F1000Prime Rep, 5:40). B cells circulate in the blood and lymphatic systems. In the lymphoid organs, a B cell encounters its cognate antigen, and together with an additional signal from a T helper cell, the B cell can differentiate into effector plasma cells. These cells secrete specific antibodies that will circulate in the blood to target and eliminate antigens or pathogens (Puri et al., 2013, Int Rev Immunol, 32(4):397-427).

In healthy individuals, immune tolerance prevents the immune system from recognizing self-antigens, thus limiting targeting and destruction of healthy cells and tissues by B, T, and myeloid cells. Autoimmune diseases, however, are characterized by a break in tolerance, wherein immune cells recognize and react to self-antigens. In such cases, B cells recognize and produce antibodies directed against self-antigens (“autoantibodies”), which are then capable of targeting cells and tissues for destruction by other components of the immune system, such as complement, cytotoxic T cells, and myeloid cells.

To detect an antigen, either pathogen-derived or self-antigen, B cells express cell surface receptors (BCRs), which are multicomponent receptors composed of a transmembrane immunoglobulin molecule (mIg) and a disulfide linked heterodimer of CD79a (Igα) and CD79b (Igβ) (Chu et al., 2001, Appl Immunohistochem Mol Morphol, June; 9(2):97-106). CD79b is selectively expressed within the B cell lineage across many differentiation states of B cells. Activation of the BCR results in multiple immune-activating consequences, including B cell differentiation, antibody and autoantibody production, cytokine production, and antigen presentation to T cells.

### SUMMARY

In one aspect, the present disclosure provides an isolated protein comprising an antigen binding domain that binds Cluster of Differentiation 79B protein (CD79b). In one embodiment, the antigen binding domain that binds CD79b comprises at least one complementarity determining region (CDR) selected from the group consisting of a heavy chain complementarity determining region (HCDR) 1, a HCDR2, a HCDR3, a light chain complementarity determining region (LCDR) 1, a LCDR2 and a LCDR 3.

In one embodiment, the HCDR1 is selected from the group consisting of SEQ ID NOs: 1, 11, 21, 31, 41, 51, 61, 71, 81, 91, 101, 111, 121, and 131; the HCDR2 is selected from the group consisting of SEQ ID NOs: 2, 12, 22, 32, 42, 52, 62, 72, 82, 92, 102, 112, 122, and 132; the HCDR3 is selected from the group consisting of SEQ ID NOs: 3, 13, 23, 33, 43, 53, 63, 73, 83, 93, 103, 113, 123, and 133; the LCDR1 is selected from the group consisting of SEQ ID NOs: 4, 14, 24, 34, 44, 54, 64, 74, 84, 94, 104, 114, 124, and 134; the LCDR2 is selected from the group consisting of SEQ ID NOs: 5, 15, 25, 35, 45, 55, 65, 75, 85, 95, 105, 115, 125, and 135; and the LCDR3 is selected from the group consisting of SEQ ID NOs: 6, 16, 26, 36, 46, 56, 66, 76, 86, 96, 106, 116, 126, and 136.

In one embodiment, the antigen binding domain that binds CD79b comprises a HCDR1, a HCDR2 and a HCDR3. In one embodiment, the antigen binding domain that binds CD79b comprises:
(a) a HCDR1 of SEQ ID NO: 1, a HCDR2 of SEQ ID NO: 2, and a HCDR3 of SEQ ID NO: 3;
(b) a HCDR1 of SEQ ID NO: 11, a HCDR2 of SEQ ID NO: 12, and a HCDR3 of SEQ ID NO: 13;
(c) a HCDR1 of SEQ ID NO: 21, a HCDR2 of SEQ ID NO: 22, and a HCDR3 of SEQ ID NO: 23;
(d) a HCDR1 of SEQ ID NO: 31, a HCDR2 of SEQ ID NO: 32, and a HCDR3 of SEQ ID NO: 33;
(e) a HCDR1 of SEQ ID NO: 41, a HCDR2 of SEQ ID NO: 42, and a HCDR3 of SEQ ID NO: 43;
(f) a HCDR1 of SEQ ID NO: 51, a HCDR2 of SEQ ID NO: 52, and a HCDR3 of SEQ ID NO: 53;
(g) a HCDR1 of SEQ ID NO: 61, a HCDR2 of SEQ ID NO: 62, and a HCDR3 of SEQ ID NO: 63;
(h) a HCDR1 of SEQ ID NO: 71, a HCDR2 of SEQ ID NO: 72, and a HCDR3 of SEQ ID NO: 73;
(i) a HCDR1 of SEQ ID NO: 81, a HCDR2 of SEQ ID NO: 82, and a HCDR3 of SEQ ID NO: 83;
(j) a HCDR1 of SEQ ID NO: 91, a HCDR2 of SEQ ID NO: 92, and a HCDR3 of SEQ ID NO: 93;
(k) a HCDR1 of SEQ ID NO: 101, a HCDR2 of SEQ ID NO: 102, a HCDR3 of SEQ ID NO: 103; a LCDR1 of SEQ ID NO: 104, a LCDR2 of SEQ ID NO:105, and a LCDR3 of SEQ ID NO: 106;
(l) a HCDR1 of SEQ ID NO: 111, a HCDR2 of SEQ ID NO: 112, a HCDR3 of SEQ ID NO: 113; a LCDR1 of SEQ ID NO: 114, a LCDR2 of SEQ ID NO:115, and a LCDR3 of SEQ ID NO: 116;
(m) a HCDR1 of SEQ ID NO: 121, a HCDR2 of SEQ ID NO: 122, a HCDR3 of SEQ ID NO: 123; a LCDR1 of SEQ ID NO: 124, a LCDR2 of SEQ ID NO:125, and a LCDR3 of SEQ ID NO: 126; or
(n) a HCDR1 of SEQ ID NO: 131, a HCDR2 of SEQ ID NO: 132, a HCDR3 of SEQ ID NO: 133; a LCDR1 of SEQ ID NO: 134, a LCDR2 of SEQ ID NO:135, and a LCDR3 of SEQ ID NO:136.

In one embodiment, the antigen binding domain that binds CD79b comprises a LCDR1, a LCDR2 and a LCDR3. In one embodiment, the antigen binding domain that binds CD79b comprises:
(a) a LCDR1 of SEQ ID NO: 4, a LCDR2 of SEQ ID NO:5, and a LCDR3 of SEQ ID NO: 6;
(b) a LCDR1 of SEQ ID NO: 14, a LCDR2 of SEQ ID NO: 15, and a LCDR3 of SEQ ID NO: 16;
(c) a LCDR1 of SEQ ID NO: 24, a LCDR2 of SEQ ID NO:25, and a LCDR3 of SEQ ID NO:26;
(d) a LCDR1 of SEQ ID NO: 34, a LCDR2 of SEQ ID NO:35, and a LCDR3 of SEQ ID NO:36;
(e) a LCDR1 of SEQ ID NO: 44, a LCDR2 of SEQ ID NO:45, and a LCDR3 of SEQ ID NO:46;
(f) a LCDR1 of SEQ ID NO: 54, a LCDR2 of SEQ ID NO:55, and a LCDR3 of SEQ ID NO:56;
(g) a LCDR1 of SEQ ID NO: 64, a LCDR2 of SEQ ID NO:65, and a LCDR3 of SEQ ID NO: 66;
(h) a LCDR1 of SEQ ID NO: 74, a LCDR2 of SEQ ID NO:75, and a LCDR3 of SEQ ID NO: 76;
(i) a LCDR1 of SEQ ID NO: 84, a LCDR2 of SEQ ID NO:85, and a LCDR3 of SEQ ID NO: 86;
(j) a LCDR1 of SEQ ID NO: 94, a LCDR2 of SEQ ID NO:95, and a LCDR3 of SEQ ID NO: 96;
(k) a LCDR1 of SEQ ID NO: 104, a LCDR2 of SEQ ID NO:5, and a LCDR3 of SEQ ID NO: 106;
(l) a LCDR1 of SEQ ID NO: 114, a LCDR2 of SEQ ID NO: 115, and a LCDR3 of SEQ ID NO:116;
(m) a LCDR1 of SEQ ID NO: 124, a LCDR2 of SEQ ID NO: 125, and a LCDR3 of SEQ ID NO:126; or
(n) a LCDR1 of SEQ ID NO: 134, a LCDR2 of SEQ ID NO: 135, and a LCDR3 of SEQ ID NO: 136.

In one embodiment, the antigen binding domain that binds CD79b comprises a HCDR1, a HCDR2, a HCDR3, LCDR1, a LCDR2, and a LCDR3. In one embodiment, the antigen binding domain that binds CD79b comprises:
(a) a HCDR1 of SEQ ID NO: 1, a HCDR2 of SEQ ID NO: 2, a HCDR3 of SEQ ID NO: 3; a LCDR1 of SEQ ID NO: 4, a LCDR2 of SEQ ID NO:5, and a LCDR3 of SEQ ID NO: 6;
(b) a HCDR1 of SEQ ID NO: 11, a HCDR2 of SEQ ID NO: 12, a HCDR3 of SEQ ID NO: 13; a LCDR1 of SEQ ID NO: 14, a LCDR2 of SEQ ID NO: 15, and a LCDR3 of SEQ ID NO: 16;
(c) a HCDR1 of SEQ ID NO: 21, a HCDR2 of SEQ ID NO: 22, a HCDR3 of SEQ ID NO: 23; a LCDR1 of SEQ ID NO: 24, a LCDR2 of SEQ ID NO:25, and a LCDR3 of SEQ ID NO:26;
(d) a HCDR1 of SEQ ID NO: 31, a HCDR2 of SEQ ID NO: 32, a HCDR3 of SEQ ID NO: 33; a LCDR1 of SEQ ID NO: 34, a LCDR2 of SEQ ID NO:35, and a LCDR3 of SEQ ID NO:36;
(e) a HCDR1 of SEQ ID NO: 41, a HCDR2 of SEQ ID NO: 42, a HCDR3 of SEQ ID NO: 43; a LCDR1 of SEQ ID NO: 44, a LCDR2 of SEQ ID NO:45, and a LCDR3 of SEQ ID NO:46;
(f) a HCDR1 of SEQ ID NO: 51, a HCDR2 of SEQ ID NO: 52, a HCDR3 of SEQ ID NO: 53; a LCDR1 of SEQ ID NO: 54, a LCDR2 of SEQ ID NO:55, and a LCDR3 of SEQ ID NO:56;
(g) a HCDR1 of SEQ ID NO: 61, a HCDR2 of SEQ ID NO: 62, a HCDR3 of SEQ ID NO: 63; a LCDR1 of SEQ ID NO: 64, a LCDR2 of SEQ ID NO:65, and a LCDR3 of SEQ ID NO:66;
(h) a HCDR1 of SEQ ID NO: 71, a HCDR2 of SEQ ID NO: 72, a HCDR3 of SEQ ID NO: 73; a LCDR1 of SEQ ID NO: 74, a LCDR2 of SEQ ID NO:75, and a LCDR3 of SEQ ID NO:76;
(i) a HCDR1 of SEQ ID NO: 81, a HCDR2 of SEQ ID NO: 82, a HCDR3 of SEQ ID NO: 83; a LCDR1 of SEQ ID NO: 84, a LCDR2 of SEQ ID NO:85, and a LCDR3 of SEQ ID NO:86;
(j) a HCDR1 of SEQ ID NO: 91, a HCDR2 of SEQ ID NO: 92, a HCDR3 of SEQ ID NO: 93; a LCDR1 of SEQ ID NO: 94, a LCDR2 of SEQ ID NO:95, and a LCDR3 of SEQ ID NO:96;
(k) a HCDR1 of SEQ ID NO: 101, a HCDR2 of SEQ ID NO: 102, a HCDR3 of SEQ ID NO: 103; a LCDR1 of SEQ ID NO: 104, a LCDR2 of SEQ ID NO:5, and a LCDR3 of SEQ ID NO: 106;
(l) a HCDR1 of SEQ ID NO: 111, a HCDR2 of SEQ ID NO: 112, a HCDR3 of SEQ ID NO: 113; a LCDR1 of SEQ ID NO: 114, a LCDR2 of SEQ ID NO:115, and a LCDR3 of SEQ ID NO: 116;
(m) a HCDR1 of SEQ ID NO: 121, a HCDR2 of SEQ ID NO: 122, a HCDR3 of SEQ ID NO: 123; a LCDR1 of SEQ ID NO: 124, a LCDR2 of SEQ ID NO: 125, and a LCDR3 of SEQ ID NO: 126; or
(n) a HCDR1 of SEQ ID NO: 131, a HCDR2 of SEQ ID NO: 132, a HCDR3 of SEQ ID NO: 133; a LCDR1 of SEQ ID NO: 134, a LCDR2 of SEQ ID NO:135, and a LCDR3 of SEQ ID NO:136.

In one embodiment, the antigen binding domain that binds CD79b comprises a heavy chain variable region (VH), wherein the VH comprises the HCDR1, HDR2 and HCDR3. In one embodiment, the VH is selected from the group consisting of SEQ ID NOs: 7, 17, 27, 37, 47, 57, 67, 77, 87, 97, 107, 117, 127, and 137.

In one embodiment, the antigen binding domain that binds CD79b comprises a light chain variable region (VL), wherein the VL comprises the LCDR1, LDR2 and LCDR3. In one embodiment, the VL is selected from the group consisting of SEQ ID NOs: 8, 18, 28, 38, 48, 58, 68, 78, 88, 98, 108, 118, 128, and 138.

In one embodiment, the VH is selected from the group consisting of SEQ ID NOs: 7, 17, 27, 37, 47, 57, 67, 77, 87, 97, 107, 117, 127, and 137 and the VL is selected from the group consisting of SEQ ID NOs: 8, 18, 28, 38, 48, 58, 68, 78, 88, 98, 108, 118, 128, and 138. In one embodiment, the antigen binding domain that binds CD79b comprises:
(a) the VH of SEQ ID NO: 7 and the VL of SEQ ID NO: 8;
(b) the VH of SEQ ID NO: 17 and the VL of SEQ ID NO: 18
(c) the VH of SEQ ID NO: 27 and the VL of SEQ ID NO: 28
(d) the VH of SEQ ID NO: 37 and the VL of SEQ ID NO: 38
(e) the VH of SEQ ID NO: 47 and the VL of SEQ ID NO: 48
(f) the VH of SEQ ID NO: 57 and the VL of SEQ ID NO: 58
(g) the VH of SEQ ID NO: 67 and the VL of SEQ ID NO: 68
(h) the VH of SEQ ID NO: 77 and the VL of SEQ ID NO: 78
(i) the VH of SEQ ID NO: 87 and the VL of SEQ ID NO: 88
(j) the VH of SEQ ID NO: 97 and the VL of SEQ ID NO: 98;
(k) the VH of SEQ ID NO: 107 and the VL of SEQ ID NO: 108;
(l) the VH of SEQ ID NO: 117 and the VL of SEQ ID NO: 118;
(m) the VH of SEQ ID NO: 127 and the VL of SEQ ID NO: 128; or
(n) the VH of SEQ ID NO: 137 and the VL of SEQ ID NO: 138.

In one embodiment, the antigen binding domain that binds CD79b is a scFv, a (scFv)₂, a Fv, a Fab, a F(ab′)₂, a Fd, a dAb or a VHH. In one embodiment, the antigen binding domain that binds CD79b is the Fab. In one embodiment, the antigen binding domain that binds CD79b is the VHH.

In one embodiment, the antigen binding domain that binds CD79b is the scFv. In one embodiment, the scFv comprises, from the N- to C-terminus, a VH, a first linker (L1) and a VL (VH-L1-VL) or the VL, the L1 and the VH (VL-L1-VH). In one embodiment, L1 comprises about 5-50 amino acids, about 5-40 amino acids, about 10-30 amino acids, or about 10-20 amino acids. In one embodiment, L1 comprises an amino acid sequence selected from the group consisting of SEQ ID NOs: 141-173. In one embodiment, the isolated protein is a monospecific protein. In one embodiment, the isolated protein is a multispecific protein. In one embodiment, the multispecific protein is a bispecific protein. In one embodiment, the multispecific protein is a trispecific protein.

In one embodiment, the isolated protein further comprises an immunoglobulin (Ig) constant region or a fragment of the Ig constant region thereof. In one embodiment, the Ig constant region comprises a Fc region. In one embodiment, the fragment of the Ig constant region comprises a CH2 domain. In one embodiment, the fragment of the Ig constant region comprises a CH3 domain. In one embodiment, the fragment of the Ig constant region comprises the CH2 domain and the CH3 domain. In one embodiment, the fragment of the Ig constant region comprises at least portion of a hinge, the CH2 domain and the CH3 domain. In one embodiment, the fragment of the Ig constant region comprises a hinge, the CH2 domain and the CH3 domain.

In one embodiment, the antigen binding domain that binds CD79b is conjugated to the N-terminus of the Ig constant region or the fragment of the Ig constant region. In one embodiment, the antigen binding domain that binds CD79b is conjugated to the C-terminus of the Ig constant region or the fragment of the Ig constant region.

In one embodiment, wherein the antigen binding domain that binds CD79b is conjugated to the Ig constant region or the fragment of the Ig constant region via a second linker (L2). In one embodiment, L2 comprises the amino acid sequence of SEQ ID NOs: 141-173.

In one embodiment, the Ig constant region or the fragment of the Ig constant region is an IgG1, an IgG2, an IgG3 or an IgG4 isotype. In one embodiment, the Ig constant region or the fragment of the Ig constant region is an IgG1 isotype.

In one embodiment, the Ig constant region or the fragment of the Ig constant region comprises at least one mutation that results in reduced binding of the protein to a Fcγ receptor (FcγR). In one embodiment, the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In one embodiment, the Ig constant region or the fragment of the Ig constant region comprises at least one mutation that results in enhanced binding of the protein to the Fey. In one embodiment, the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In one embodiment, the FcγR is FcγRI, FcγRIIA, FcγRIIB or FcγRIII, or any combination thereof.

In one embodiment, the Ig constant region of the fragment of the Ig constant region comprises at least one mutation that modulates a half-life of the protein. In one embodiment, the at least one mutation that modulates the half-life of the protein is selected from the group consisting of H435A, P257I/N434H, D376V/N434H, M252Y/S254T/T256E/H433K/N434F, T308P/N434A and H435R, wherein residue numbering is according to the EU index.

In one embodiment, the protein comprises at least one mutation in a CH3 domain of the Ig constant region. In one embodiment, the at least one mutation in the CH3 domain of the Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In one embodiment, the antigen binding domain that binds CD79b comprises a heavy chain and light chain, wherein the heavy chain comprises the VH and the light chain comprises the VL. In one embodiment, the HC is selected from the group consisting of SEQ ID NOs: 9, 19, 29, 39, 49, 59, 69, 79, 89, 99, 109, 119, 129, or 139 the LC is selected from the group consisting of SEQ ID NOs: 10, 20, 30, 40, 50, 60, 70, 80, 90, 100, 110, 120, 130, or 140.

In one embodiment, the antigen binding domain that binds CD79b comprises:
(a) the HC of SEQ ID NO: 9 and the LC of SEQ ID NO: 10;
(b) the HC of SEQ ID NO: 19 and the LC of SEQ ID NO: 20;
(c) the HC of SEQ ID NO: 29 and the LC of SEQ ID NO: 30;
(d) the HC of SEQ ID NO: 39 and the LC of SEQ ID NO: 40;
(e) the HC of SEQ ID NO: 49 and the LC of SEQ ID NO: 50;
(f) the HC of SEQ ID NO: 59 and the LC of SEQ ID NO: 60;
(g) the HC of SEQ ID NO: 69 and the LC of SEQ ID NO: 70;
(h) the HC of SEQ ID NO: 79 and the LC of SEQ ID NO: 80;
(i) the HC of SEQ ID NO: 89 and the LC of SEQ ID NO: 90;
(j) the HC of SEQ ID NO: 99 and the LC of SEQ ID NO: 100;
(k) the HC of SEQ ID NO: 109 and the LC of SEQ ID NO: 110;
(l) the HC of SEQ ID NO: 119 and the LC of SEQ ID NO: 120;
(m) the HC of SEQ ID NO: 129 and the LC of SEQ ID NO: 130; or
(n) the HC of SEQ ID NO: 139 and the LC of SEQ ID NO: 140.

In one aspect, the disclosure provides an immunoconjugate comprising an isolated protein of the disclosure conjugated to a therapeutic agent or an imaging agent. In one aspect, the disclosure provides a pharmaceutical composition comprising the isolated protein of the disclosure and a pharmaceutically acceptable carrier.

In one aspect, the disclosure provides polynucleotide encoding the isolated protein of the disclosure. In one aspect, the disclosure provides vector comprising a polynucleotide of the disclosure. In one aspect, the disclosure provides a host cell comprising a vector of the disclosure.

In one aspect, the disclosure provides a method of producing the isolated protein of the disclosure, comprising culturing the host cell of the disclosure in conditions that the protein is expressed, and recovering the protein produced by the host cell.

In one aspect, the disclosure provides a method comprising administering a therapeutically effective amount of the antigen binding domain that binds CD79b of the isolated protein of the disclosure, the immunoconjugate of the disclosure, or the pharmaceutical composition of the disclosure to a subject having an autoimmune disease.

In one aspect, the disclosure provides a method of treating an autoimmune disease in a subject. In one embodiment, the method comprises administering a therapeutically effective amount of the antigen binding domain that binds CD79b of the isolated protein of the disclosure, the immunoconjugate of the disclosure, or the pharmaceutical composition of the disclosure to a subject for a time sufficient to treat the autoimmune disease.

In one aspect, the disclosure provides a method of preventing an autoimmune disease in a subject. In one embodiment, the method comprises administering a therapeutically effective amount of the antigen binding domain that binds CD79b of the isolated protein of the disclosure, the immunoconjugate of the disclosure, or the pharmaceutical composition of the disclosure to a subject for a time sufficient to prevent the autoimmune disease.

In one embodiment, the autoimmune disease is associated with or characterized by dysregulation of B cells, autoreactive B cells, or the presence of autoantibodies. In one embodiment, the autoimmune disease is selected from the group consisting of Systemic lupus erythematosus (SLE), Sjögren's syndrome (SjS), Rheumatoid arthritis, Autoimmune myopathies, Type I diabetes, Addison disease, Pernicious anemia, Autoimmune hepatitis, Primary biliary cholangitis (PBC), Autoimmune pancreatitis, Celiac disease, Focal segmental glomerulosclerosis, Primary membranous nephropathy, Ovarian insufficiency, Autoimmune orchitis, Dry eye disease, Idiopathic interstitial pneumonias, Thyroid disease (eg Grave's), Systemic sclerosis (Scleroderma), Myasthenic syndromes, Autoimmune encephalitis, Bullous skin diseases, TTP, ITP, AIHA, Anca vasculitis, Myocarditis/dilatory CM, NMOSD, Maternal-fetal alloimmunity, Maternal-fetal autoimmunity, Anti-cardiolipin/antiphospholipid syndrome, Hypergammaglobulinemia, Transplant-associated ID, Multifocal motor neuropathy.

In one aspect, the disclosure provides a method of modulating B cell activation in a subject. In one embodiment, the method comprises administering a therapeutically effective amount of the antigen binding domain that binds CD79b of the isolated protein of the disclosure, the immunoconjugate of the disclosure, or the pharmaceutical composition of the disclosure to the subject for a time sufficient to modulate B cell activation.

In one aspect, the disclosure provides a method of inhibiting aberrant B cell activation in a subject. In one embodiment, the method comprises administering a therapeutically effective amount of the antigen binding domain that binds CD79b of the isolated protein of the disclosure, the immunoconjugate of the disclosure, or the pharmaceutical composition of the disclosure to the subject for a time sufficient to inhibit aberrant B cell activation.

In one aspect, the disclosure provides a kit comprising isolated protein of the disclosure, the immunoconjugate of the disclosure, or the pharmaceutical composition of the disclosure.

In one aspect, the disclosure provides an anti-idiotypic antibody binding to the isolated protein of the disclosure.

### DETAILED DESCRIPTION

The disclosed methods may be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended, and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Activation,” “stimulation,” “activated,” or “stimulated” refer to induction of a change in the biologic state of a cell resulting in expression of activation markers, cytokine production, proliferation or mediating cytotoxicity of target cells. Cells may be activated by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or natural killer (NK) cell proliferation and/or upregulation or downregulation of key molecules.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antibody-dependent cellular cytotoxicity”, “antibody-dependent cell-mediated cytotoxicity” or “ADCC” refers to the mechanism of inducing cell death that depends upon the interaction of antibody-coated target cells with effector cells possessing lytic activity, such as NK cells, monocytes, macrophages and neutrophils via Fc gamma receptors (FcγR) expressed on effector cells.

“Antibody-dependent cellular phagocytosis” or “ADCP” refers to the mechanism of elimination of antibody-coated target cells by internalization by phagocytic cells, such as macrophages or dendritic cells.

“Antigen” refers to any molecule (e.g., protein, peptide, polysaccharide, glycoprotein, glycolipid, nucleic acid, portions thereof, or combinations thereof) capable of being bound by an antigen binding domain. Antigens may be expressed by genes, synthetized, or purified from biological samples such as a tissue sample, a tumor sample, a cell or a fluid with other biological components, organisms, subunits of proteins/antigens, and killed or inactivated whole cells or lysates.

“Antigen binding fragment” or “antigen binding domain” refers to a portion of the protein that binds an antigen. Antigen binding fragments may be synthetic, enzymatically obtainable or genetically engineered polypeptides and include portions of an immunoglobulin that bind an antigen, such as VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each HC is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes: IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Chimeric antigen receptor” (CAR) as used herein is defined as a cell-surface receptor comprising an extracellular target-binding domain, a transmembrane domain and an intracellular signaling domain, all in a combination that is not naturally found together on a single protein. This includes receptors wherein the extracellular domain and the intracellular signaling domain are not naturally found together on a single receptor protein. CARs are intended primarily for use with lymphocyte such as T cells and NK cells.

“Complement-dependent cytotoxicity” or “CDC”, refers to the mechanism of inducing cell death in which the Fc effector domain of a target-bound protein binds and activates complement component C1q which in turn activates the complement cascade leading to target cell death. Activation of complement may also result in deposition of complement components on the target cell surface that facilitate CDC by binding complement receptors (e.g., CR3) on leukocytes

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g. Lefranc et al. (2003) Dev Comp Immunol 27: 55-77; Honegger and Pluckthun, J Mol Biol (2001) 309:657-70; International ImMunoGeneTics (IMGT) database; Web resources, www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT orAbM, unless otherwise explicitly stated in the specification.

“Decrease,” “lower,” “lessen,” “reduce,” or “abate” refers generally to the ability of a test molecule to mediate a reduced response (i.e., downstream effect) when compared to the response mediated by a control or a vehicle. Exemplary responses are T cell expansion, T cell activation or T-cell mediated tumor cell killing or binding of a protein to its antigen or receptor, and enhanced binding to a Fcγ or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Domain Antibody,” “dAb,” or “dAb fragment” refers to an antibody fragment composed of either VH and the VL domains from a single arm of the antibody.

“Differentiation” refers to a method of decreasing the potency or proliferation of a cell or moving the cell to a more developmentally restricted state.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Enhance,” “promote,” “increase,” “expand” or “improve” refers generally to the ability of a test molecule to mediate a greater response (i.e., downstream effect) when compared to the response mediated by a control or a vehicle. Exemplary responses are T cell expansion, T cell activation or T-cell mediated tumor cell killing or binding of a protein to its antigen or receptor, and enhanced binding to a Fcγ or enhanced Fc effector functions such as enhanced ADCC, CDC and/or ADCP. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Expansion” refers to the outcome of cell division and cell death.

“Express” and “expression” refers the to the well-known transcription and translation occurring in cells or in vitro. The expression product, e.g., the protein, is thus expressed by the cell or in vitro and may be an intracellular, extracellular or a transmembrane protein.

“Expression vector” refers to a vector that can be utilized in a biological system or in a reconstituted biological system to direct the translation of a polypeptide encoded by a polynucleotide sequence present in the expression vector. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

“dAb” or “dAb fragment” refers to an antibody fragment composed of a VH domain (Ward et al., Nature 341:544 546 (1989)).

“Fab” or “Fab fragment” refers to an antibody fragment composed of VH, CH1, VL and CL domains.

“F(ab′)₂” or “F(ab′)₂ fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody.

“Full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Genetic modification” refers to the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to the polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

“Heterologous” refers to two or more polynucleotides or two or more polypeptides that are not found in the same relationship to each other in nature.

“Heterologous polynucleotide” refers to a non-naturally occurring polynucleotide that encodes two or more neoantigens as described herein.

“Heterologous polypeptide” refers to a non-naturally occurring polypeptide comprising two or more neoantigen polypeptides as described herein.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“In combination with” means that two or more therapeutic agents are be administered to a subject together in a mixture, concurrently as single agents or sequentially as single agents in any order.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Cluster of Differentiation CD79B protein” or “CD79b” refers to a known protein which is also called CD79b. The amino acid sequences of the various isoforms are retrievable from GenBank accession numbers AAH32651.1, EAW94232.1, AAH02975.2, NP_000617.1, and NP_001035022.1. The amino acid sequence of the full length CD79b sequence is shown below. The sequence includes the extracellular domain (residues 29-159) and the cytoplasmic domain (residues 181-229).

(SEQ ID NO: 177)
MARLALSPVPSHWMVALLLLLSAEPVPAARSEDRYRNPKGSACSRIWQS
PRFIARKRGFTVKMHCYMNSASGNVSWLWKQEMDENPQQLKLEKGRMEE
SQNESLATLTIQGIRFEDNGIYFCQQKCNNTSEVYQGCGTELRVMGFST
LAQLKQRNTLKDGIIMIQTLLIILFIIVPIFLLLDKDDSKAGMEEDHTY
EGLDIDQTATYEDIVTLRTGEVKWSVGEHPGQE

“Modulate” refers to either enhanced or decreased ability of a test molecule to mediate an enhanced or a reduced response (i.e., downstream effect) when compared to the response mediated by a control or a vehicle.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Minibody” to refers to scFv fragments which are linked via CH3 domains.

“Multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

“Natural killer cell” and “NK cell” are used interchangeably and synonymously herein. NK cell refers to a differentiated lymphocyte with a CD16⁺CD56⁺ and/or CD57⁺ TCR⁻ phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

“Operatively linked” and similar phrases, when used in reference to nucleic acids or amino acids, refers to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA) and in some instances to the production of a polypeptide (i.e., expression of the open reading frame). “Operatively linked peptide” refers to a peptide in which the functional domains of the peptide are placed with appropriate distance from each other to impart the intended function of each domain.

“Pharmaceutical combination” refers to a combination of two or more active ingredients administered either together or separately.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Prevent,” “preventing,” “prevention,” or “prophylaxis” of a disease or disorder means preventing a disorder from occurring in a subject.

“Proliferation” refers to an increase in cell division, either symmetric or asymmetric division of cells.

“Promoter” refers to the minimal sequences required to initiate transcription. Promoter may also include enhancers or repressor elements which enhance or suppress transcription, respectively.

“Protein” or “polypeptide” are used interchangeably herein are refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Recombinant” refers to polynucleotides, polypeptides, vectors, viruses and other macromolecules that are prepared, expressed, created or isolated by recombinant means.

“Regulatory element” refers to any cis- or trans acting genetic element that controls some aspect of the expression of nucleic acid sequences.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻² M or less, typically with a K_{D} that is at least one hundred fold less than its K_{D} for binding to a non-specific antigen (e.g., BSA, casein). In the context of the CD79b antigens described here, “specific binding” refers to binding of the proteinaceous molecule to the CD79b antigen without detectable binding to a wild-type protein the antigen is a variant of.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8⁺ cytotoxic T cell response. Also included are “regulatory T cells” or “Tregs” which refer to T cells that suppress an abnormal or excessive immune response and play a role in immune tolerance. Tregs are typically transcription factor Foxp3-positive CD4⁺ T cells and can also include transcription factor Foxp3-negative regulatory T cells that are IL-10-producing CD4⁺ T cells.

“Therapeutically effective amount” or “effective amount” as used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics include, for example, improved wellbeing of the patient, reduction of a tumor burden, arrested or slowed growth of a tumor, and/or absence of metastasis of cancer cells to other locations in the body.

“Transduction” refers to the introduction of a foreign nucleic acid into a cell using a viral vector.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Mutations in the Ig constant regions are referred to as follows: L351Y_F405A_Y407V refers to L351Y, F405A and Y407V mutations in one immunoglobulin constant region.

L351Y_F405A_Y407V/T394W refers to L351Y, F405A and Y407V mutations in the first Ig constant region and T394W mutation in the second Ig constant region, which are present in one multimeric protein.

CD79b Binding Domains

Antibodies or antigen binding domains that target CD79B are therefore capable of delivering agents to the BCR complex. As such, CD79B targeting molecules are useful in the generation of bispecific agents to recruit naturally occurring inhibitory proteins to the BCR complex, to inhibit aberrant B cell activation. For example, a bispecific dual-affinity retargeting (DART) molecule with antigen binding domains recognizing both CD79B and the inhibitory Fc gamma receptor, CD32B, inhibits B cell activation, monitored by reduced B cell proliferation and immunoglobulin secretion. Similar bispecific molecules recognizing both CD79b and CD32B delayed the onset and reduced disease severity in a preclinical model of autoimmune arthritis, indicating that CD79b-mediated recruitment of inhibitory proteins to the BCR could provide therapeutic benefit to patients with autoimmune diseases (Veri et al., 2010, Arthritis & Rheumatism, 62: 1933-1943). CD79B-targeting bispecific antibodies or antigen binding domains could also target other inhibitory molecules expressed on the surface of B cells, including members of the Siglec family, such as CD22 or Siglec-10, which have also been demonstrated to inhibit BCR-mediated signaling (reviewed in Duan & Paulson 2020, Ann. Rev Immunol 38(1):365-369).

CD79B-targeting antibodies or antigen binding domains could also be useful in treating various forms of cancer. For example, polatuzumab vedotin, an anti-CD79b antibody-drug-conjugate is approved for treatment of diffuse large B-cell lymphoma patients (DLBCL) (reviewed in Walji 2020, PMID: 32700586; DOI: 10.1080/17474086.2020.1795828). Additionally, engineered T cells expressing chimeric antigen receptors (CAR T cells) that bind to CD79B eliminated CD79B-expressing B cell lymphoma cells in vitro and in vivo (Ding 2020, PMID: 32495161 DOI: 10.1007/s11523-020-00729-7; Jiang 2020, PMID: 31624374 DOI: 10.1038/s41375-019-0607-5; Ormhøj2019, PMID: 31439577 PMCID: PMC6891163 DOI: 10.1158/1078-0432.CCR-19-1337).

Compositions of Matter

Antigen Binding Domains that Bind CD79b

The disclosure provides antigen binding domains that bind CD79b, monospecific and multispecific proteins comprising the antigen binding domains that bind CD79b, chimeric antigen receptors (CAR) comprising the antigen binding domains that bind CD79b, polynucleotides encoding the foregoing, vectors, host cells and methods of making and using the foregoing.

The disclosure provides an isolated protein comprising an antigen binding domain that binds Cluster of Differentiation CD79B protein (CD79b). In one embodiment, the isolated protein comprising an antigen binding domain that binds Cluster of Differentiation CD79B protein (CD79b), wherein the antigen binding domain that binds CD79b comprises a heavy chain complementarity determining region (HCDR) 1 of SEQ ID NOs: 1, 11, 21, 31, 41, 51, 61, 71, 81, 91, 101, 111, 121, or 131.

In one embodiment, the isolated protein comprising an antigen binding domain that binds Cluster of Differentiation CD79B protein (CD79b), wherein the antigen binding domain that binds CD79b comprises a HCDR2 of SEQ ID NOs: 2, 12, 22, 32, 42, 52, 62, 72, 82, 92, 102, 112, 122 or 132.

In one embodiment, the isolated protein comprising an antigen binding domain that binds Cluster of Differentiation CD79B protein (CD79b), wherein the antigen binding domain that binds CD79b comprises a HCDR3 of SEQ ID NOs: 3, 13, 23, 33, 43, 53, 63, 73, 83, 93, 103, 113, 123, or 133.

In one embodiment, the isolated protein comprising an antigen binding domain that binds Cluster of Differentiation CD79B protein (CD79b), wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In one embodiment, the isolated protein comprising an antigen binding domain that binds Cluster of Differentiation CD79B protein (CD79b), wherein the antigen binding domain that binds CD79b comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In one embodiment, the isolated protein comprising an antigen binding domain that binds Cluster of Differentiation CD79B protein (CD79b), wherein the antigen binding domain that binds CD79b comprises a light chain complementarity determining region (LCDR) 1 of SEQ ID NOs: 4, 14, 24, 34, 44, 54, 64, 74, 84, 94, 104, 114, 124, or 134.

In one embodiment, the isolated protein comprising an antigen binding domain that binds Cluster of Differentiation CD79B protein (CD79b), wherein the antigen binding domain that binds CD79b comprises a LCDR2 of SEQ ID NOs: 5, 15, 25, 35, 45, 55, 65, 75, 85, 95, 105, 115, 125, or 135.

In one embodiment, the isolated protein comprising an antigen binding domain that binds Cluster of Differentiation CD79B protein (CD79b), wherein the antigen binding domain that binds CD79b comprises a LCDR3 of SEQ ID NOs: 6, 16, 26, 36, 46, 56, 66, 76, 86, 96, 106, 116, 126, or 136.

In one embodiment, the isolated protein comprising an antigen binding domain that binds Cluster of Differentiation CD79B protein (CD79b), wherein the antigen binding domain that binds CD79b comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the isolated protein comprising an antigen binding domain that binds Cluster of Differentiation CD79B protein (CD79b), wherein the antigen binding domain that binds CD79b comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In one embodiment, the isolated protein comprising an antigen binding domain that binds Cluster of Differentiation CD79B protein (CD79b), wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the isolated protein comprising an antigen binding domain that binds Cluster of Differentiation CD79B protein (CD79b), wherein the antigen binding domain that binds CD79b comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In one embodiment, the isolated protein comprising an antigen binding domain that binds CD79b, wherein the antigen binding domain that binds CD79b comprises the VH of SEQ ID NOs:7, 17, 27, 37, 47, 57, 67, 77, 87, 97, 107, 117, 127, or 137.

In one embodiment, the isolated protein comprising an antigen binding domain that binds CD79b, wherein the antigen binding domain that binds CD79b comprises the VL of SEQ ID NOs: 8, 18, 28, 38, 48, 58, 68, 78, 88, 98, 108, 118, 128, or 138.

In one embodiment, the isolated protein comprising an antigen binding domain that binds CD79b, wherein the antigen binding domain that binds CD79b comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In one embodiment, the isolated protein comprising an antigen binding domain that binds CD79b, wherein the antigen binding domain that binds CD79b comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In one embodiment, the isolated protein comprising an antigen binding domain that binds CD79b, wherein the antigen binding domain that binds CD79b comprises the heavy chain (HC) of SEQ ID NOs: 9, 19, 29, 39, 49, 59, 69, 79, 89, 99, 109, 119, 129, or 139.

In one embodiment, the isolated protein comprising an antigen binding domain that binds CD79b, wherein the antigen binding domain that binds CD79b comprises the light chain (LC) of SEQ ID NOs: 10, 20, 30, 40, 50, 60, 70, 80, 90, 100, 110, 120, 130, or 140.

In one embodiment, the isolated protein comprising an antigen binding domain that binds CD79b, wherein the antigen binding domain that binds CD79b comprises the HC of SEQ ID NOs: 9, 19, 29, 39, 49, 59, 69, 79, 89, 99, 109, 119, 129, or 139 and the LC of SEQ ID NOs: 10, 20, 30, 40, 50, 60, 70, 80, 90, 100, 110, 120, 130, or 140.

In one embodiment, the isolated protein comprising an antigen binding domain that binds CD79b, wherein the antigen binding domain that binds CD79b comprises the HC and LC of:
SEQ ID NOs: 9 and 10, respectively;
SEQ ID NOs: 19 and 20, respectively;
SEQ ID NOs: 29 and 30, respectively;
SEQ ID NOs: 39 and 40, respectively;
SEQ ID NOs: 49 and 50, respectively;
SEQ ID NOs: 59 and 60, respectively;
SEQ ID NOs: 69 and 70, respectively;
SEQ ID NOs: 79 and 80, respectively;
SEQ ID NOs: 89 and 90, respectively;
SEQ ID NOs: 99 and 100, respectively;
SEQ ID NOs: 109 and 110, respectively;
SEQ ID NOs: 119 and 120, respectively;
SEQ ID NOs: 129 and 130, respectively; or
SEQ ID NOs: 139 and 140, respectively.

In some embodiments, the antigen binding domain that binds CD79b is a scFv.

In some embodiments, the antigen binding domain that binds CD79b is a (scFv)₂.

In some embodiments, the antigen binding domain that binds CD79b is a Fv.

In some embodiments, the antigen binding domain that binds CD79b is a Fab.

In some embodiments, the antigen binding domain that binds CD79b is a F(ab′)₂.

In some embodiments, the antigen binding domain that binds CD79b is a Fd.

In some embodiments, the CD79b antigen binding domain is a dAb.

In some embodiments, the CD79b antigen binding domain is a VHH.

Multispecific Proteins

In some embodiments, the disclosure provides a multispecific protein (e.g., a mulitspecific antibody) comprising an antigen binding domain that binds CD79b. For example, in one embodiment, the antigen binding domains that bind CD79b can be incorporated into the Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

In one embodiment, the multispecific protein comprises an antigen binding domain that binds Cluster of Differentiation CD79B protein (CD79b). In one embodiment, the isolated protein comprising an antigen binding domain that binds Cluster of Differentiation CD79B protein (CD79b), wherein the antigen binding domain that binds CD79b comprises a heavy chain complementarity determining region (HCDR) 1 of SEQ ID NOs: 1, 11, 21, 31, 41, 51, 61, 71, 81, 91, 101, 111, 121, or 131.

In one embodiment, the multispecific protein comprises an antigen binding domain that binds Cluster of Differentiation CD79B protein (CD79b), wherein the antigen binding domain that binds CD79b comprises a HCDR2 of SEQ ID NOs: 2, 12, 22, 32, 42, 52, 62, 72, 82, 92, 102, 112, 122, or 132.

In one embodiment, the multispecific protein comprises an antigen binding domain that binds Cluster of Differentiation CD79B protein (CD79b), wherein the antigen binding domain that binds CD79b comprises a HCDR3 of SEQ ID NOs: 3, 13, 23, 33, 43, 53, 63, 73, 83, 93, 103, 113, 123, or 133.

In one embodiment, the multispecific protein comprises an antigen binding domain that binds Cluster of Differentiation CD79B protein (CD79b), wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In one embodiment, the multispecific protein comprises an antigen binding domain that binds Cluster of Differentiation CD79B protein (CD79b), wherein the antigen binding domain that binds CD79b comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In one embodiment, the multispecific protein comprises an antigen binding domain that binds Cluster of Differentiation CD79B protein (CD79b), wherein the antigen binding domain that binds CD79b comprises a light chain complementarity determining region (LCDR) 1 of SEQ ID NOs: 4, 14, 24, 34, 44, 54, 64, 74, 84, 94, 104, 114, 124, or 134.

In one embodiment, the multispecific protein comprises an antigen binding domain that binds Cluster of Differentiation CD79B protein (CD79b), wherein the antigen binding domain that binds CD79b comprises a LCDR2 of SEQ ID NOs: 5, 15, 25, 35, 45, 55, 65, 75, 85, 95, 105, 115, 125, or 135.

In one embodiment, the multispecific protein comprises an antigen binding domain that binds Cluster of Differentiation CD79B protein (CD79b), wherein the antigen binding domain that binds CD79b comprises a LCDR3 of SEQ ID NOs: 6, 16, 26, 36, 46, 56, 66, 76, 86, 96, 106, 116, 126, or 136.

In one embodiment, the multispecific protein comprises an antigen binding domain that binds Cluster of Differentiation CD79B protein (CD79b), wherein the antigen binding domain that binds CD79b comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the multispecific protein comprises an antigen binding domain that binds Cluster of Differentiation CD79B protein (CD79b), wherein the antigen binding domain that binds CD79b comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In one embodiment, the multispecific protein comprises an antigen binding domain that binds Cluster of Differentiation CD79B protein (CD79b), wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the multispecific protein comprises an antigen binding domain that binds Cluster of Differentiation CD79B protein (CD79b), wherein the antigen binding domain that binds CD79b comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In one embodiment, the multispecific protein comprises an antigen binding domain that binds CD79b, wherein the antigen binding domain that binds CD79b comprises the VH of SEQ ID NOs:7, 17, 27, 37, 47, 57, 67, 77, 87, 97, 107, 117, 127, or 137.

In one embodiment, the multispecific protein comprises an antigen binding domain that binds CD79b, wherein the antigen binding domain that binds CD79b comprises the VL of SEQ ID NOs: 8, 18, 28, 38, 48, 58, 68, 78, 88, 98, 108, 118, 128, or 138.

In one embodiment, the multispecific protein comprises an antigen binding domain that binds CD79b, wherein the antigen binding domain that binds CD79b comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In one embodiment, the multispecific protein comprises an antigen binding domain that binds CD79b, wherein the antigen binding domain that binds CD79b comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In one embodiment, the antigen binding domain that bind CD79b is incorporated into a Dual Affinity Retargeting Technology (DART) protein. In one aspect, an antigen binding domain that binds CD79B can be incorporated into a DART protein. In certain aspects a DART protein comprises two heterodimerized Fv fragments that form two unique antigen-binding sites. In one embodiment, the DART comprises a first Fv comprising a VH derived from a first antigen binding domain (VH1) and a VL derived from a second antigen binding domain (VL2). In one embodiment, the DART comprises a second Fv comprising a VH derived from the second antigen binding domain (VH2) and a VL derived from the first antigen binding domain (VL1).

In one embodiment, the DART protein comprises an antigen binding domain that binds Cluster of Differentiation CD79B protein (CD79b). In one embodiment, the isolated protein comprising an antigen binding domain that binds Cluster of Differentiation CD79B protein (CD79b), wherein the antigen binding domain that binds CD79b comprises a heavy chain complementarity determining region (HCDR) 1 of SEQ ID NOs: 1, 11, 21, 31, 41, 51, 61, 71, 81, 91, 101, 111, 121, or 131.

In one embodiment, the DART protein comprises an antigen binding domain that binds Cluster of Differentiation CD79B protein (CD79b), wherein the antigen binding domain that binds CD79b comprises a HCDR2 of SEQ ID NOs: 2, 12, 22, 32, 42, 52, 62, 72, 82, 92, 102, 112, 122, or 132.

In one embodiment, the DART protein comprises an antigen binding domain that binds Cluster of Differentiation CD79B protein (CD79b), wherein the antigen binding domain that binds CD79b comprises a HCDR3 of SEQ ID NOs: 3, 13, 23, 33, 43, 53, 63, 73, 83, 93, 103, 113, 123, or 133.

In one embodiment, the DART protein comprises an antigen binding domain that binds Cluster of Differentiation CD79B protein (CD79b), wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In one embodiment, the DART protein comprises an antigen binding domain that binds Cluster of Differentiation CD79B protein (CD79b), wherein the antigen binding domain that binds CD79b comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In one embodiment, the DART protein comprises an antigen binding domain that binds Cluster of Differentiation CD79B protein (CD79b), wherein the antigen binding domain that binds CD79b comprises a light chain complementarity determining region (LCDR) 1 of SEQ ID NOs: 4, 14, 24, 34, 44, 54, 64, 74, 84, 94, 104, 114, 124, or 134.

In one embodiment, the DART protein comprises an antigen binding domain that binds Cluster of Differentiation CD79B protein (CD79b), wherein the antigen binding domain that binds CD79b comprises a LCDR2 of SEQ ID NOs: 5, 15, 25, 35, 45, 55, 65, 75, 85, 95, 105, 115, 125, or 135.

In one embodiment, the DART protein comprises an antigen binding domain that binds Cluster of Differentiation CD79B protein (CD79b), wherein the antigen binding domain that binds CD79b comprises a LCDR3 of SEQ ID NOs: 6, 16, 26, 36, 46, 56, 66, 76, 86, 96, 106, 116, 126, or 136.

In one embodiment, the DART protein comprises an antigen binding domain that binds Cluster of Differentiation CD79B protein (CD79b), wherein the antigen binding domain that binds CD79b comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the DART protein comprises an antigen binding domain that binds Cluster of Differentiation CD79B protein (CD79b), wherein the antigen binding domain that binds CD79b comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In one embodiment, the DART protein comprises an antigen binding domain that binds Cluster of Differentiation CD79B protein (CD79b), wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the DART protein comprises an antigen binding domain that binds Cluster of Differentiation CD79B protein (CD79b), wherein the antigen binding domain that binds CD79b comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In one embodiment, the DART protein comprises an antigen binding domain that binds CD79b, wherein the antigen binding domain that binds CD79b comprises the VH of SEQ ID NOs:7, 17, 27, 37, 47, 57, 67, 77, 87, 97, 107, 117, 127, or 137.

In one embodiment, the DART protein comprises an antigen binding domain that binds CD79b, wherein the antigen binding domain that binds CD79b comprises the VL of SEQ ID NOs: 8, 18, 28, 38, 48, 58, 68, 78, 88, 98, 108, 118, 128, or 138.

In one embodiment, the DART protein comprises an antigen binding domain that binds CD79b, wherein the antigen binding domain that binds CD79b comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In one embodiment, the DART protein comprises an antigen binding domain that binds CD79b, wherein the antigen binding domain that binds CD79b comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

CD79b Binding scFvs

Any of the VH and the VL domains identified herein that bind CD79b may be engineered into scFv format in either VH-linker-VL or VL-linker-VH orientation. Any of the VH and the VL domains identified herein may also be used to generate sc(Fv)₂ structures, such as VH-linker-VL-linker-VL-linker-VH, VH-linker-VL-linker-VH-linker-VL. VH-linker-VH-linker-VL-linker-VL. VL-linker-VH-linker-VH-linker-VL. VL-linker-VH-linker-VL-linker-VH or VL-linker-VL-linker-VH-linker-VH. VH and the VL domains identified herein may be incorporated into a scFv format and the binding and thermostability of the resulting scFv to CD79b may be assessed using known methods. Binding may be assessed using ProteOn XPR36, Biacore 3000 or KinExA instrumentation, ELISA or competitive binding assays known to those skilled in the art. Binding may be evaluated using purified scFvs or E. coli supernatants or lysed cells containing the expressed scFv. The measured affinity of a test scFv to CD79b may vary if measured under different conditions (e.g., osmolarity, pH). Thus, measurements of affinity and other binding parameters (e.g., K_{D}, Kₒₙ, K_{off}) are typically made with standardized conditions and standardized buffers. Thermostability may be evaluated by heating the test scFv at elevated temperatures, such as at 50° C., 55° C. or 60° C. for a period of time, such as 5 minutes (min), 10 min, 15 min, 20 min, 25 min or 30 min and measuring binding of the test scFv to CD79b. The scFvs retaining comparable binding to CD79b when compared to a non-heated scFv sample are referred to as being thermostable.

In recombinant expression systems, the linker is a peptide linker and may include any naturally occurring amino acid. Exemplary amino acids that may be included into the linker are Gly, Ser, Pro, Thr, Glu, Lys, Arg, Ile, Leu, His and The. The linker should have a length that is adequate to link the VH and the VL in such a way that they form the correct conformation relative to one another so that they retain the desired activity, such as binding to CD79b.

The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that may be used are Gly rich linkers, Gly and Ser containing linkers, Gly and Ala containing linkers, Ala and Ser containing linkers, and other flexible linkers.

Other linker sequences may include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Additional linkers are described for example in Int. Pat. Publ. No. WO2019/060695.

In some embodiments, the scFv comprises, from the N- to C-terminus, a VH, a first linker (L1) and a VL (VH-L1-VL). In some embodiments, the scFv comprises, from the N- to C-terminus, the VL, the L1 and the VH (VL-L1-VH).

In some embodiments, the scFV comprises a heavy chain complementarity determining region (HCDR) 1 of SEQ ID NOs: 1, 11, 21, 31, 41, 51, 61, 71, 81, 91, 101, 111, 121, or 131.

In some embodiments, the scFV comprises a HCDR2 of SEQ ID NOs: 2, 12, 22, 32, 42, 52, 62, 72, 82, 92, 102, 112, 122, or 132.

In some embodiments, the scFV comprises a HCDR3 of SEQ ID NOs: 3, 13, 23, 33, 43, 53, 63, 73, 83, 93, 103, 113, 123, or 133.

In some embodiments, the scFV comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In some embodiments, the scFV comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In some embodiments, the scFV comprises a light chain complementarity determining region (LCDR) 1 of SEQ ID NOs: 4, 14, 24, 34, 44, 54, 64, 74, 84, 94, 104, 114, 124, or 134.

In some embodiments, the scFV comprises a LCDR2 of SEQ ID NOs: 5, 15, 25, 35, 45, 55, 65, 75, 85, 95, 105, 115, 125, or 135.

In some embodiments, the scFV comprises a LCDR3 of SEQ ID NOs: 6, 16, 26, 36, 46, 56, 66, 76, 86, 96, 106, 116, 126, or 136.

In some embodiments, the scFV comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the scFV comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In some embodiments, the scFV comprises HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the scFV comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In some embodiments, the scFV comprises the VH of SEQ ID NOs: 7, 17, 27, 37, 47, 57, 67, 77, 87, 97, 107, 117, 127, or 137.

In some embodiments, the scFV comprises the VL of SEQ ID NOs: 8, 18, 28, 38, 48, 58, 68, 78, 88, 98, 108, 118, 128, or 138.

In some embodiments, the scFV comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In some embodiments, the scFV comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

Other Antigen Binding Domains that Bind CD79b

Any of the VH and the VL domains identified herein that bind CD79b may also be engineered into Fab, F(ab′)₂, Fd or Fv format and their binding to CD79b may be assessed using the assays described herein.

In some embodiments, the Fab comprises a heavy chain complementarity determining region (HCDR) 1 of SEQ ID NOs: 1, 11, 21, 31, 41, 51, 61, 71, 81, 91, 101, 111, 121, or 131.

In some embodiments, the Fab comprises a HCDR2 of SEQ ID NOs: 2, 12, 22, 32, 42, 52, 62, 72, 82, 92, 102, 112, 122, or 132.

In some embodiments, the Fab comprises a HCDR3 of SEQ ID NOs: 3, 13, 23, 33, 43, 53, 63, 73, 83, 93, 103, 113, 123, or 133.

In some embodiments, the Fab comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In some embodiments, the Fab comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In some embodiments, the Fab comprises a light chain complementarity determining region (LCDR) 1 of SEQ ID NOs: 4, 14, 24, 34, 44, 54, 64, 74, 84, 94, 104, 114, 124, or 134.

In some embodiments, the Fab comprises a LCDR2 of SEQ ID NOs: 5, 15, 25, 35, 45, 55, 65, 75, 85, 95, 105, 115, 125, or 135.

In some embodiments, the Fab comprises a LCDR3 of SEQ ID NOs: 6, 16, 26, 36, 46, 56, 66, 76, 86, 96, 106, 116, 126, or 136.

In some embodiments, the Fab comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the Fab comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In some embodiments, the Fab comprises HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the Fab comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In some embodiments, the Fab comprises the VH of SEQ ID NOs: 7, 17, 27, 37, 47, 57, 67, 77, 87, 97, 107, 117, 127, or 137.

In some embodiments, the Fab comprises the VL of SEQ ID NOs: 8, 18, 28, 38, 48, 58, 68, 78, 88, 98, 108, 118, 128, or 138.

In some embodiments, the Fab comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In some embodiments, the Fab comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In some embodiments, the F(ab′)₂ comprises a heavy chain complementarity determining region (HCDR) 1 of SEQ ID NOs: 1, 11, 21, 31, 41, 51, 61, 71, 81, 91, 101, 111, 121, or 131.

In some embodiments, the F(ab′)₂ comprises a HCDR2 of SEQ ID NOs: 2, 12, 22, 32, 42, 52, 62, 72, 82, 92, 102, 112, 122, or 132.

In some embodiments, the F(ab′)₂ comprises a HCDR3 of SEQ ID NOs: 3, 13, 23, 33, 43, 53, 63, 73, 83, 93, 103, 113, 123, or 133.

In some embodiments, the F(ab′)₂ comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In some embodiments, the F(ab′)₂ comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In some embodiments, the F(ab′)₂ comprises a light chain complementarity determining region (LCDR) 1 of SEQ ID NOs: 4, 14, 24, 34, 44, 54, 64, 74, 84, 94, 104, 114, 124, or 134.

In some embodiments, the F(ab′)₂ comprises a LCDR2 of SEQ ID NOs: 5, 15, 25, 35, 45, 55, 65, 75, 85, 95, 105, 115, 125, or 135.

In some embodiments, the F(ab′)₂ comprises a LCDR3 of SEQ ID NOs: 6, 16, 26, 36, 46, 56, 66, 76, 86, 96, 106, 116, 126, or 136.

In some embodiments, the F(ab′)₂ comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the F(ab′)₂ comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In some embodiments, the F(ab′)₂ comprises HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the F(ab′)₂ comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In some embodiments, the F(ab′)₂ comprises the VH of SEQ ID NOs: 7, 17, 27, 37, 47, 57, 67, 77, 87, 97, 107, 117, 127, or 137.

In some embodiments, the F(ab′)₂ comprises the VL of SEQ ID NOs: 8, 18, 28, 38, 48, 58, 68, 78, 88, 98, 108, 118, 128, or 138.

In some embodiments, the F(ab′)₂ comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In some embodiments, the F(ab′)₂ comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In some embodiments, the Fv comprises a heavy chain complementarity determining region (HCDR) 1 of SEQ ID NOs: 1, 11, 21, 31, 41, 51, 61, 71, 81, 91, 101, 111, 121, or 131.

In some embodiments, the Fv comprises a HCDR2 of SEQ ID NOs: 2, 12, 22, 32, 42, 52, 62, 72, 82, 92, 102, 112, 122, or 132.

In some embodiments, the Fv comprises a HCDR3 of SEQ ID NOs: 3, 13, 23, 33, 43, 53, 63, 73, 83, 93, 103, 113, 123, or 133.

In some embodiments, the Fv comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In some embodiments, the Fv comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In some embodiments, the Fv comprises a light chain complementarity determining region (LCDR) 1 of SEQ ID NOs: 4, 14, 24, 34, 44, 54, 64, 74, 84, 94, 104, 114, 124, or 134.

In some embodiments, the Fv comprises a LCDR2 of SEQ ID NOs: 5, 15, 25, 35, 45, 55, 65, 75, 85, 95, 105, 115, 125, or 135.

In some embodiments, the Fv comprises a LCDR3 of SEQ ID NOs: 6, 16, 26, 36, 46, 56, 66, 76, 86, 96, 106, 116, 126, or 136.

In some embodiments, the Fv comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the Fv comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In some embodiments, the Fv comprises HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the Fv comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In some embodiments, the Fv comprises the VH of SEQ ID NOs: 7, 17, 27, 37, 47, 57, 67, 77, 87, 97, 107, 117, 127, or 137.

In some embodiments, the Fv comprises the VL of SEQ ID NOs: 8, 18, 28, 38, 48, 58, 68, 78, 88, 98, 108, 118, 128, or 138.

In some embodiments, the Fv comprises the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In some embodiments, the Fv comprises the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

Homologous Antigen Binding Domains and Antigen Binding Domains with Conservative Substitutions

Variants of the antigen binding domains that bind CD79b are within the scope of the disclosure. For example, variants may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29 or more amino acid substitutions in the antigen binding domain that bind CD79b as long as they retain or have improved functional properties when compared to the parent antigen binding domains. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding domains that bind CD79b of the disclosure. In some embodiments, the variation is in the framework regions. In some embodiments, variants are generated by conservative substitutions.

In some embodiments, the antigen binding domain that binds CD79b comprises an HCDR1 which is at least 80% identical to the HCDR1 of SEQ ID NOs: 1, 11, 21, 31, 41, 51, 61, 71, 81, 91, 101, 111, 121, or 131. In some embodiments, the identity is at least 85%. In some embodiments, the identity is at least 90%. In some embodiments, the identity is at least 91%. In some embodiments, the identity is at least 91%. In some embodiments, the identity is at least 92%. In some embodiments, the identity is at least 93%. In some embodiments, the identity is at least 94%. In some embodiments, the identity is at least 95%. In some embodiments, the identity is at least 96%. In some embodiments, the identity is at least 97%. In some embodiments, the identity is at least 98%. In some embodiments, the identity is at least 99%.

In some embodiments, the antigen binding domain that binds CD79b comprises an HCDR2 which is at least 80% identical to the HCDR2 of SEQ ID NO: 2, 12, 22, 32, 42, 52, 62, 72, 82, 92, 102, 112, 122, or 132. In some embodiments, the identity is at least 85%. In some embodiments, the identity is at least 90%. In some embodiments, the identity is at least 91%. In some embodiments, the identity is at least 91%. In some embodiments, the identity is at least 92%. In some embodiments, the identity is at least 93%. In some embodiments, the identity is at least 94%. In some embodiments, the identity is at least 95%. In some embodiments, the identity is at least 96%. In some embodiments, the identity is at least 97%. In some embodiments, the identity is at least 98%. In some embodiments, the identity is at least 99%.

In some embodiments, the antigen binding domain that binds CD79b comprises an HCDR3 which is at least 80% identical to the HCDR3 of SEQ ID NOs: 3, 13, 23, 33, 43, 53, 63, 73, 83, 93, 103, 113, 123, or 133. In some embodiments, the identity is at least 85%. In some embodiments, the identity is at least 90%. In some embodiments, the identity is at least 91%. In some embodiments, the identity is at least 91%. In some embodiments, the identity is at least 92%. In some embodiments, the identity is at least 93%. In some embodiments, the identity is at least 94%. In some embodiments, the identity is at least 95%. In some embodiments, the identity is at least 96%. In some embodiments, the identity is at least 97%. In some embodiments, the identity is at least 98%. In some embodiments, the identity is at least 99%.

In some embodiments, the antigen binding domain that binds CD79b comprises an LCDR1 which is at least 80% identical to the LCDR1 of SEQ ID NOs: 4, 14, 24, 34, 44, 54, 64, 74, 84, 94, 104, 114, 124, or 134. In some embodiments, the identity is at least 85%. In some embodiments, the identity is at least 90%. In some embodiments, the identity is at least 91%. In some embodiments, the identity is at least 91%. In some embodiments, the identity is at least 92%. In some embodiments, the identity is at least 93%. In some embodiments, the identity is at least 94%. In some embodiments, the identity is at least 95%. In some embodiments, the identity is at least 96%. In some embodiments, the identity is at least 97%. In some embodiments, the identity is at least 98%. In some embodiments, the identity is at least 99%.

In some embodiments, the antigen binding domain that binds CD79b comprises an LCDR2 which is at least 80% identical to the LCDR2 of SEQ ID NO: 5, 15, 25, 35, 45, 55, 65, 75, 85, 95, 105, 115, 125, or 135. In some embodiments, the identity is at least 85%. In some embodiments, the identity is at least 90%. In some embodiments, the identity is at least 91%. In some embodiments, the identity is at least 91%. In some embodiments, the identity is at least 92%. In some embodiments, the identity is at least 93%. In some embodiments, the identity is at least 94%. In some embodiments, the identity is at least 95%. In some embodiments, the identity is at least 96%. In some embodiments, the identity is at least 97%. In some embodiments, the identity is at least 98%. In some embodiments, the identity is at least 99%.

In some embodiments, the antigen binding domain that binds CD79b comprises an LCDR3 which is at least 80% identical to the LCDR3 of SEQ ID NOs: 6, 16, 26, 36, 46, 56, 66, 76, 86, 96, 106, 116, 126, or 136. In some embodiments, the identity is at least 85%. In some embodiments, the identity is at least 90%. In some embodiments, the identity is at least 91%. In some embodiments, the identity is at least 91%. In some embodiments, the identity is at least 92%. In some embodiments, the identity is at least 93%. In some embodiments, the identity is at least 94%. In some embodiments, the identity is at least 95%. In some embodiments, the identity is at least 96%. In some embodiments, the identity is at least 97%. In some embodiments, the identity is at least 98%. In some embodiments, the identity is at least 99%.

Also provided are antigen binding domains that bind CD79b comprising the VH and the VL which are at least 80% identical to the VH and VL of SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In some embodiments, the identity is at least 85%. In some embodiments, the identity is at least 90%.

In some embodiments, the identity is at least 91%. In some embodiments, the identity is at least 91%.

In some embodiments, the identity is at least 92%. In some embodiments, the identity is at least 93%.

In some embodiments, the identity is at least 94%. In some embodiments, the identity is at least 95%.

In some embodiments, the identity is at least 96%. In some embodiments, the identity is at least 97%.

In some embodiments, the identity is at least 98%. In some embodiments, the identity is at least 99%.

The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences.

The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at www_gcg_com), using either a Blossum 62 matrix or a PAM250 matrix, and a gap weight of 16, 14, 12, 10, 8, 6, or 4 and a length weight of 1, 2, 3, 4, 5, or 6.

In some embodiments, variant antigen binding domains that bind CD79b comprise one or two conservative substitutions in any of the CDR regions, while retaining desired functional properties of the parent antigen binding fragments that bind CD79b.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

Methods of Generating Antigen Binding Fragment that Bind CD79b

Antigen binding domains that bind CD79b provided in the disclosure may be generated using various technologies. For example, the hybridoma method of Kohler and Milstein may be used to identify VH/VL pairs that bind CD79b. In the hybridoma method, a mouse or other host animal, such as a hamster, rat or chicken is immunized with human and/or cyno CD79b, followed by fusion of spleen cells from immunized animals with myeloma cells using standard methods to form hybridoma cells. Colonies arising from single immortalized hybridoma cells may be screened for production of the antibodies containing the antigen binding domains that bind CD79b with desired properties, such as specificity of binding, cross-reactivity or lack thereof, affinity for the antigen, and any desired functionality.

Antigen binding domains that bind CD79b generated by immunizing non-human animals may be humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding domains may be further optimized to improve their selectivity or affinity to a desired antigen by incorporating altered framework support residues to preserve binding affinity (backmutations) by techniques such as those described in Int. Patent Publ. Nos. WO1990/007861 and WO1992/22653, or by introducing variation at any of the CDRs for example to improve affinity of the antigen binding domain.

Transgenic animals, such as mice, rat or chicken carrying human immunoglobulin (Ig) loci in their genome may be used to generate antigen binding fragments that bind CD79b, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (www_regeneron_com), Harbour Antibodies (www_harbourantibodies_com), Open Monoclonal Technology, Inc. (OMT) (www_omtinc_net), KyMab (www_kymab_com), Trianni (www_trianni_com) and Ablexis (www_ablexis_com) may be engaged to provide human antibodies directed against a selected antigen using technologies as described above.

Antigen binding domains that bind CD79b may be selected from a phage display library, where the phage is engineered to express human immunoglobulins or portions thereof such as Fabs, single chain antibodies (scFv), or unpaired or paired antibody variable regions. The antigen binding domains that bind CD79b may be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries may be screened for phage binding to human and/or cyno CD79b and the obtained positive clones may be further characterized, the Fabs isolated from the clone lysates, and converted to scFvs or other configurations of antigen binding fragments.

Preparation of immunogenic antigens and expression and production of antigen binding domains of the disclosure may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

Conjugation to Half-Life Extending Moieties

The antigen binding domains that bind CD79b of the disclosure may be conjugated to a half-life extending moiety. Exemplary half-life extending moieties are albumin, albumin variants, albumin-binding proteins and/or domains, transferrin and fragments and analogues thereof, immunoglobulins (Ig) or fragments thereof, such as Fc regions. Amino acid sequences of the aforementioned half-life extending moieties are known. Ig or fragments thereof include all isotypes, i.e., IgG1, IgG2, IgG3, IgG4, IgM, IgA and IgE.

Additional half-life extending moieties that may be conjugated to the antigen binding domains that bind CD79b of the disclosure include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the antigen binding domains that bind CD79b of the disclosure and may be generated by standard cloning and expression techniques. Alternatively, well known chemical coupling methods may be used to attach the moieties to recombinantly produced antigen binding domains that bind CD79b of the disclosure.

A pegyl moiety may for example be conjugated to the antigen binding domain that bind CD79b of the disclosure by incorporating a cysteine residue to the C-terminus of the antigen binding domain that bind CD79b of the disclosure, or engineering cysteines into residue positions that face away from the CD79b binding site and attaching a pegyl group to the cysteine using well known methods.

In some embodiments, the antigen binding fragment that binds CD79b is conjugated to a half-life extending moiety.

In some embodiments, the half-life extending moiety is an immunoglobulin (Ig), a fragment of the Ig, an Ig constant region, a fragment of the Ig constant region, a Fc region, transferrin, albumin, an albumin binding domain or polyethylene glycol. In some embodiments, the half-life extending moiety is an Ig constant region.

In some embodiments, the half-life extending moiety is the Ig.

In some embodiments, the half-life extending moiety is the fragment of the Ig.

In some embodiments, the half-life extending moiety is the Ig constant region.

In some embodiments, the half-life extending moiety is the fragment of the Ig constant region.

In some embodiments, the half-life extending moiety is the Fc region.

In some embodiments, the half-life extending moiety is albumin.

In some embodiments, the half-life extending moiety is the albumin binding domain.

In some embodiments, the half-life extending moiety is transferrin.

In some embodiments, the half-life extending moiety is polyethylene glycol.

The antigen binding domains that bind CD79b conjugated to a half-life extending moiety may be evaluated for their pharmacokinetic properties utilizing known in vivo models.

Isotypes, Allotypes and Fc Engineering

The Ig constant region or the fragment of the Ig constant region, such as the Fc region present in the proteins of the disclosure may be of any allotype or isotype.

In some embodiments, the Ig constant region or the fragment of the Ig constant region is an IgG1 isotype.

In some embodiments, the Ig constant region or the fragment of the Ig constant region is an IgG2 isotype.

In some embodiments, the Ig constant region or the fragment of the Ig constant region is an IgG3 isotype.

In some embodiments, the Ig constant region or the fragment of the Ig constant region is an IgG4 isotype.

The Ig constant region or the fragment of the Ig constant region may be of any allotype. It is expected that allotype has no influence on properties of the Ig constant region, such as binding or Fc-mediated effector functions. Immunogenicity of therapeutic proteins comprising Ig constant regions of fragments thereof is associated with increased risk of infusion reactions and decreased duration of therapeutic response (Baert et al., (2003) N. Engl. J Med. 348:602-08). The extent to which therapeutic proteins comprising Ig constant regions of fragments thereof induce an immune response in the host may be determined in part by the allotype of the Ig constant region (Stickler et al., (2011) Genes and Immunity 12:213-21). Ig constant region allotype is related to amino acid sequence variations at specific locations in the constant region sequences of the antibody. Table 1 shows select IgG1, IgG2 and IgG4 allotypes.

TABLE 1

| | Amino acid residue at position of | | | | | | | |
|---|---|---|---|---|---|---|---|---|
| | diversity (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| Glm(17) | | | | | K | E | M | A |
| Glm(17,1) | | | | | K | D | L | A |

C-terminal lysine (CTL) may be removed from the Ig constant region by endogenous circulating carboxypeptidases in the blood stream (Cai et al., (2011) Biotechnol. Bioeng. 108:404-412). During manufacturing, CTL removal may be controlled to less than the maximum level by control of concentration of extracellular Zn²⁺, EDTA or EDTA—Fe³⁺ as described in U.S. Patent Publ. No. US20140273092. CTL content of proteins may be measured using known methods.

In some embodiments, the antigen binding fragment that binds CD79b conjugated to the Ig constant region has a C-terminal lysine content from about 10% to about 90%. In some embodiments, the C-terminal lysine content is from about 20% to about 80%. In some embodiments, the C-terminal lysine content is from about 40% to about 70%. In some embodiments, the C-terminal lysine content is from about 55% to about 70%. In some embodiments, the C-terminal lysine content is about 60%. Fc region mutations may be made to the antigen binding domains that bind CD79b conjugated to the Ig constant region or to the fragment of the Ig constant region to modulate their effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), inhibitory FcγRIIb and/or to FcRn.

In some embodiments, the antigen binding domain that binds CD79b conjugated to the Ig constant region or the fragment of the Ig constant region comprises at least one mutation in the Ig constant region or in the fragment of the Ig constant region.

In some embodiments, the at least one mutation is in the Fc region.

In some embodiments, the antigen binding domain that binds CD79b conjugated to the Ig constant region or to the fragment of the Ig constant region comprises at least one, two, three, four, five, six, seven, eight, nine, ten, eleven, twelve, thirteen, fourteen or fifteen mutations in the Fc region.

In some embodiments, the antigen binding domain that binds CD79b conjugated to the Ig constant region or to the fragment of the Ig constant region comprises at least one mutation in the Fc region that modulates binding of the antibody to FcRn.

Fc positions that may be mutated to modulate half-life (e.g. binding to FcRn) include positions 250, 252, 253, 254, 256, 257, 307, 376, 380, 428, 434 and 435. Exemplary mutations that may be made singularly or in combination are mutations T250Q, M252Y, I253A, S254T, T256E, P257I, T307A, D376V, E380A, M428L, H433K, N434S, N434A, N434H, N434F, H435A and H435R. Exemplary singular or combination mutations that may be made to increase the half-life are mutations M428L/N434S, M252Y/S254T/T256E, T250Q/M428L, N434A and T307A/E380A/N434A.

Exemplary singular or combination mutations that may be made to reduce the half-life are mutations H435A, P257I/N434H, D376V/N434H, M252Y/S254T/T256E/H433K/N434F, T308P/N434A and H435R.

In some embodiments, the antigen binding domain that binds CD79b conjugated to the Ig constant region or to the fragment of the Ig constant region comprises M252Y/S254T/T256E mutation.

In some embodiments, the antigen binding domain that binds CD79b conjugated to the Ig constant region or to the fragment of the Ig constant region comprises at least one mutation in the Fc region that reduces binding of the protein to an activating Fey receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding of the protein to the activating FcγR and subsequently to reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in proteins with reduced ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

An exemplary mutation that results in proteins with reduced CDC is a K322A mutation.

Well-known S228P mutation may be made in IgG4 to enhance IgG4 stability.

In some embodiments, the antigen binding domain that binds CD79b conjugated to the Ig constant region or to the fragment of the Ig constant region comprises at least one mutation selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, K322, A330S and P331S.

In some embodiments, the antigen binding domain that binds CD79b conjugated to the Ig constant region or to the fragment of the Ig constant region comprises L234A/L235A/D265S mutation.

In some embodiments, the antigen binding domain that binds CD79b conjugated to the Ig constant region or to the fragment of the Ig constant region comprises L234A/L235A mutation.

In some embodiments, the antigen binding domain that binds CD79b conjugated to the Ig constant region or to the fragment of the Ig constant region comprises at least one mutation in the Fc region that enhances binding of the protein to an Fcγ receptor (FcγR) and/or enhances Fc effector functions such as C1q binding, complement dependent cytotoxicity (CDC), antibody-dependent cell-mediated cytotoxicity (ADCC) and/or phagocytosis (ADCP).

Fc positions that may be mutated to increase binding of the protein to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index).

Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination mutations that result in proteins with increased ADCC or ADCP are a S239D/I332E, S298A/E333A/K334A, F243L/R292P/Y300L, F243L/R292P/Y300L/P396L, F243L/R292P/Y300L/V305I/P396L and G236A/S239D/I332E.

Fe positions that may be mutated to enhance CDC include positions 267, 268, 324, 326, 333, 345 and 430. Exemplary mutations that may be made singularly or in combination are S267E, F1268F, S324T, K326A, K326W, E333A, E345K, E345Q, E345R, E345Y, E430S, E430F and E430T.

Exemplary combination mutations that result in proteins with increased CDC are K326A/E333A, K326W/E333A, H268F/S324T, S267E/H268F, S267E/S324T and S267E/H268F/S324T.

The specific mutations described herein are mutations when compared to the IgG1, IgG2 and IgG4 wild-type amino acid sequences of SEQ ID NOs:174, 175, and 176, respectively.

-wild-type IgG1
SEQ ID NO: 174
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVM
HEALHNHYTQKSLSLSPGK
-wild-type IgG2
SEQ ID NO: 175
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMTKNQVSLTCLVK
GFYPSDISVEWESNGQPENNYKTTPPMLDSDGSFFLYSKLTVDKSRWQQGNVFSCSVMHEA
LHNHYTQKSLSLSPGK
-wild-type IgG4
SEQ ID NO: 176
ASTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPEFLGGPSVFLFPPKP
KDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEAL
HNHYTQKSLSLSLGK

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

Proteins Comprising the Antigen Binding Domains that Bind CD79b of the Disclosure

The antigen binding domains that bind CD79b of the disclosure may be engineered into monospecific or multispecific proteins of various designs using standard methods. The disclosure also provides a monospecific protein comprising the antigen binding domain that binds CD79b of the disclosure. In some embodiments, the monospecific protein is an antibody.

While not being limited by this approach, in general when constructing antibodies as multi-specific antibodies, the binding domain modules to each target (first, second, third etc) are optional built from scFv, Fab, Fab′, F(ab′)2, Fv, variable domain (e.g. VH or VL), diabody, minibody or full length antibodies. For example, each said binding domain or module is created in one or more of the following non-limiting formats wherein binding domains comprising variable domains, and/or full length antibodies, and/or antibody fragments, are operatively linked in series to generate multi-specific antibodies.

In one embodiment there is provided a multi-specific antibody comprising at least one first antibody-derived binding domain targeting CD79b and which is operatively linked to at least one second antibody binding domain targeting a second epitope. Optionally, the binding domains comprise at least one or more VH and cognate VL binding domain, or one or more VH-CH1-CH2-CH2 and cognate VL-CL binding domain, or one or more antibody fragment binding domains.

The disclosure also provides a multispecific protein comprising the antigen binding domain that binds CD79b of the disclosure.

In some embodiments, the multispecific protein is bispecific.

In some embodiments, the multispecific protein is trispecific.

In some embodiments, the multispecific protein is tetraspecific.

In some embodiments, the multispecific protein is monovalent for binding to CD79b.

In some embodiments, the multispecific protein is bivalent for binding to CD79b.

The disclosure also provides an isolated multispecific protein comprising a first antigen binding domain that binds CD79b and a second antigen binding domain that binds a second antigen. In some embodiments, the first antigen binding domain that binds CD79b and/or the second antigen binding domain that binds the second antigen comprise a scFv, a (scFv)₂, a Fv, a Fab, a F(ab′)₂, a Fd, a dAb or a VHH.

In some embodiments, the first antigen binding domain that binds CD79b and/or the second antigen binding domain that binds the second antigen comprise the Fab.

In some embodiments, the first antigen binding domain that binds CD79b and/or the second antigen binding domain that binds the second antigen comprise the F(ab′)₂.

In some embodiments, the first antigen binding domain that binds CD79b and/or the second antigen binding domain that binds the second antigen comprise the VHH.

In some embodiments, the first antigen binding domain that binds CD79b and/or the second antigen binding domain that binds the second antigen comprise the Fv.

In some embodiments, the first antigen binding domain that binds CD79b and/or the second antigen binding domain that binds the second antigen comprise the Fd.

In some embodiments, the first antigen binding domain that binds CD79b and/or the second antigen binding domain that binds the second antigen comprise the scFv.

In some embodiments, the scFv comprises, from the N- to C-terminus, a VH, a first linker (L1) and a VL (VH-L1-VL) or the VL, the L1 and the VH (VL-L1-VH). In some embodiments, the L1 comprises about 5-50 amino acids. In some embodiments, the L1 comprises about 5-40 amino acids.

In some embodiments, the L1 comprises about 10-30 amino acids. In some embodiments, the L1 comprises about 10-20 amino acids. In some embodiments, the L1 comprises the amino acid sequence of SEQ ID NOs: 141-173.

In some embodiments, the first antigen binding domain that binds CD79b comprises a HCDR1 of SEQ ID NOs: 1, 11, 21, 31, 41, 51, 61, 71, 81, 91, 101, 111, 121, or 131.

In some embodiments, the first antigen binding domain that binds CD79b comprises a HCDR2 of SEQ ID NOs: 2, 12, 22, 32, 42, 52, 62, 72, 82, 92, 102, 112, 122, or 132.

In some embodiments, the first antigen binding domain that binds CD79b comprises a HCDR3 of SEQ ID NOs: 3, 13, 23, 33, 43, 53, 63, 73, 83, 93, 103, 113, 123, or 133.

In some embodiments, the first antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In some embodiments, the first antigen binding domain that binds CD79b comprises a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In some embodiments, the first antigen binding domain that binds CD79b comprises a light chain complementarity determining region (LCDR) 1 of SEQ ID NOs: 4, 14, 24, 34, 44, 54, 64, 74, 84, 94, 104, 114, 124, or 134.

In some embodiments, the first antigen binding domain that binds CD79b comprises a LCDR2 of SEQ ID NOs: 5, 15, 25, 35, 45, 55, 65, 75, 85, 95, 105, 115, 125, or 135.

In some embodiments, the first antigen binding domain that binds CD79b comprises LCDR3 of SEQ ID NOs: 6, 16, 26, 36, 46, 56, 66, 76, 86, 96, 106, 116, 126, or 136.

In some embodiments, the first antigen binding domain that binds CD79b comprises a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the first antigen binding domain that binds CD79b comprises a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In some embodiments, the first antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the first antigen binding domain that binds CD79b comprises a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In some embodiments, the first antigen binding domain that binds CD79b comprises a VH of SEQ ID NOs: 7, 17, 27, 37, 47, 57, 67, 77, 87, 97, 107, 117, 127, or 137.

In some embodiments, the first antigen binding domain that binds CD79b comprises a VL of SEQ ID NOs: 8, 18, 28, 38, 48, 58, 68, 78, 88, 98, 108, 118, 128, or 138.

In some embodiments, the first antigen binding domain that binds CD79b comprises a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In some embodiments, the first antigen binding domain that binds CD79b comprises a VH and a VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In some embodiments, the first antigen binding domain that binds CD79b is conjugated to a first immunoglobulin (Ig) constant region or a fragment of the first Ig constant region and/or the second antigen binding domain that binds the second antigen is conjugated to a second immunoglobulin (Ig) constant region or a fragment of the second Ig constant region.

In some embodiments, the fragment of the first Ig constant region and/or the fragment of the second Ig constant region comprises a Fc region.

In some embodiments, the fragment of the first Ig constant region and/or the fragment of the second Ig constant region comprises a CH2 domain.

In some embodiments, the fragment of the first Ig constant region and/or the fragment of the second Ig constant region comprises a CH3 domain.

In some embodiments, the fragment of the first Ig constant region and/or the fragment of the second Ig constant region comprises the CH2 domain and the CH3 domain.

In some embodiments, the fragment of the first Ig constant region and/or the fragment of the second Ig constant region comprises at least portion of a hinge, the CH2 domain and the CH3 domain.

In some embodiments, the fragment of the Ig constant region comprises the hinge, the CH2 domain and the CH3 domain.

In some embodiments, the multispecific protein further comprises a second linker (L2) between the first antigen binding domain that binds CD79b and the first Ig constant region or the fragment of the first Ig constant region and the second antigen binding domain that binds the second antigen and the second Ig constant region or the fragment of the second Ig constant region.

In some embodiments, the L2 comprises the amino acid sequence of SEQ ID NOs: 141-173.

In some embodiments, the first Ig constant region or the fragment of the first Ig constant region and the second Ig constant region or the fragment of the second Ig constant region is an IgG1, an IgG2, and IgG3 or an IgG4 isotype.

In some embodiments, the first Ig constant region or the fragment of the first Ig constant region and the second Ig constant region or the fragment of the second Ig constant region is an IgG1 isotype.

In some embodiments, the first Ig constant region or the fragment of the first Ig constant region and the second Ig constant region or the fragment of the second Ig constant region is an IgG2 isotype.

In some embodiments, the first Ig constant region or the fragment of the first Ig constant region and the second Ig constant region or the fragment of the second Ig constant region is an IgG3 isotype.

In some embodiments, the first Ig constant region or the fragment of the first Ig constant region and the second Ig constant region or the fragment of the second Ig constant region is an IgG4 isotype.

The first Ig constant region or the fragment of the first Ig constant region and the second Ig constant region or the fragment of the second Ig constant region can further be engineered as described herein.

In some embodiments, the first Ig constant region or the fragment of the first Ig constant region and the second Ig constant region or the fragment of the second Ig constant region comprises at least one mutation that results in reduced binding of the multispecific protein to a FcγR.

In some embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR).

In some embodiments, the at least one mutation that results in enhanced binding of the multispecific protein to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index.

In some embodiments, the FcγR is FcγRI, FcγRIIA, FcγRIIB or FcγRIII, or any combination thereof.

In some embodiments, the first Ig constant region or the fragment of the first Ig constant region and the second Ig constant region or the fragment of the second Ig constant region comprises at least one mutation that modulates a half-life of the multispecific protein.

In some embodiments, the at least one mutation that modulates the half-life of the multispecific protein is selected from the group consisting of H435A, P257I/N434H, D376V/N434H, M252Y/S254T/T256E/H433K/N434F, T308P/N434A and H435R, wherein residue numbering is according to the EU index.

In some embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region.

In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the first Ig constant region or the fragment of the first Ig constant region and the second Ig constant region or the fragment of the second Ig constant region comprise the following mutations L235A_L235A_D265S_T350V_L351Y_F405A_Y407V in the first Ig constant region and L235A_L235A_D265S_T350V_T366L_K392L_T394W in the second Ig constant region; or L235A_L235A_D265S_T350V_T366L_K392L_T394W in the first Ig constant region and L235A_L235A_D265S_T350V_L351Y_F405A_Y407V in the second Ig constant region.

Generation of Multispecific Proteins that Comprise Antigen Binding Fragments that Bind CD79b

The antigen binding fragments that bind CD79b of the disclosure may be engineered into multispecific antibodies which are also encompassed within the scope of the invention.

The antigen binding fragments that bind CD79b may be engineered into full length multispecific antibodies which are generated using Fab arm exchange, in which substitutions are introduced into two monospecific bivalent antibodies within the Ig constant region CH3 domain which promote Fab arm exchange in vitro. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g. Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region.

Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chai heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the antigen binding domains that bind CD79b can be incorporated include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (Medlmmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fe-DART) (MacroGenics) and Dual(ScFv)₂-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

The antigen binding domains that bind CD79b of the disclosure may also be engineered into multispecific proteins which comprise three polypeptide chains. In such designs, at least one antigen binding domain is in the form of a scFv. Exemplary designs include (in which “1” indicates the first antigen binding domain, “2” indicates the second antigen binding domain and “3” indicates the third antigen binding domain:
Design 1: Chain A) scFv1-CH2-CH3; Chain B) VL2-CL; Chain C) VH2-CH1-hinge-CH2-CH3
Design 2: Chain A) scFv1-hinge-CH2-CH3; Chain B) VL2-CL; Chain C) VH2-CH1-hinge-CH2-CH3
Design 3: Chain A) scFv1-CH1-hinge-CH2-CH3; Chain B) VL2-CL; Chain C) VH2-CH1-hinge-CH2-CH3
Design 4: Chain A) CH2-CH3-scFv1; Chain B) VL2-CL; Chain C) VH2-CH1-hinge-CH2-CH3 CH3 engineering may be incorporated to the Designs 1-4, such as mutations
L351Y_F405A_Y407V/T394W, T366I_K392M_T394W/F405A_Y407V,
T366L_K392M_T394W/F405A_Y407V, L351Y_Y407A/T366A_K409F,
L351Y_Y407A/T366V_K409F, Y407A/T366A_K409F, or
T350V_L351Y_F405A_Y407V/T350V_T366L_K392L_T394W as described in US2012/0149876 or US2013/0195849 (Zymeworks).

Conjugation to Immunoglobulin (Ig) Constant Regions or Fragments of the Ig Constant Regions

In some embodiments, the antigen binding domains that bind CD79b of the disclosure are conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The antigen binding domains that bind CD79b of the disclosure may be engineered into conventional full length antibodies using standard methods. The full length antibodies comprising the antigen binding domain that binds CD79b may further be engineered as described herein.

In some embodiments, an immunoglobulin heavy chain constant region is comprised of subdomains CH1, hinge, CH2 and CH3. In some embodiments, the CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. In some embodiments, the Ig Fc region comprises at least the CH2 and the CH3 domains of the Ig constant region, and therefore comprises at least a region from about A231 to K447 of Ig heavy chain constant region.

The invention also provides an antigen binding domain that binds CD79b conjugated to an immunoglobulin (Ig) constant region or a fragment of the Ig constant region.

In some embodiments, the Ig constant region is a heavy chain constant region.

In some embodiments, the Ig constant region is a light chain constant region.

In some embodiments, the fragment of the Ig constant region comprises a Fc region.

In some embodiments, the fragment of the Ig constant region comprises a CH2 domain.

In some embodiments, the fragment of the Ig constant region comprises a CH3 domain.

In some embodiments, the fragment of the Ig constant region comprises the CH2 domain and the CH3 domain.

In some embodiments, the fragment of the Ig constant region comprises at least portion of a hinge, the CH2 domain and the CH3 domain. Portion of the hinge refers to one or more amino acid residues of the Ig hinge.

In some embodiments, the fragment of the Ig constant region comprises the hinge, the CH2 domain and the CH3 domain.

In some embodiments, the antigen binding domain that binds CD79b is conjugated to the N-terminus of the Ig constant region or the fragment of the Ig constant region.

In some embodiments, the antigen binding domain that binds CD79b is conjugated to the C-terminus of the Ig constant region or the fragment of the Ig constant region.

In some embodiments, the antigen binding domain that binds CD79b is conjugated to the Ig constant region or the fragment of the Ig constant region via a second linker (L2).

In some embodiments, the L2 comprises the amino acid sequence of SEQ ID NOs: 141-173.

The antigen binding domains that binds CD79b of the disclosure conjugated to Ig constant region or the fragment of the Ig constant region may be assessed for their functionality using several known assays. Binding to CD79b may be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCP.

ADCC may be assessed using an in vitro assay using CD79b expressing cells as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP may be evaluated by using monocyte-derived macrophages as effector cells and any CD79b expressing cells as target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods.

CDC of cells may be measured for example by plating Daudi cells at 1×10⁵ cells/well (50 μL/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C.

Percentage (%) lysed cells may be detected as % propidium iodide stained cells in FACS assay using standard methods.

Glycoengineering

The ability of the antigen binding domain that binds CD79b conjugated to the Ig constant region or to the fragment of the Ig constant region to mediate ADCC can be enhanced by engineering the Ig constant region or the fragment of the Ig constant region oligosaccharide component. Human IgG1 or IgG3 are N-glycosylated at Asn297 with the majority of the glycans in the well-known biantennary G0, G0F, G1, G1F, G2 or G2F forms. Ig constant region containing proteins may be produced by non-engineered CHO cells typically have a glycan fucose content of about at least 85%. The removal of the core fucose from the biantennary complex-type oligosaccharides attached to the antigen binding domain that binds CD79b conjugated to the Ig constant region or to the fragment of the Ig constant region enhances the ADCC of the protein via improved FcγRIIIa binding without altering antigen binding or CDC activity. Such proteins can be achieved using different methods reported to lead to the successful expression of relatively high defucosylated immunoglobulins bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4): 405-415, 2010; PMID:20562582), application of a rat hybridoma cell line YB2/0 as the host cell line (Shinkawa et al., J Biol Chem 278:3466-3473, 2003), introduction of small interfering RNA specifically against the a 1,6-fucosyltrasferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments, the antigen binding domain that binds CD79b conjugated to the Ig constant region or to the fragment of the Ig constant region of the disclosure has a biantennary glycan structure with fucose content of about between 1% to about 15%, for example about 15%, 14%, 13%, 12%, 11% 10%, 9%, 8%, 7%, 6%, 5%, 4%, 3%, 2% or 1%. In some embodiments, the antigen binding domain that binds CD79b conjugated to the Ig constant region or to the fragment of the Ig constant region has a glycan structure with fucose content of about 50%, 40%, 45%, 40%, 35%, 30%, 25%, or 20%.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to the antigen binding domain that bind CD79b conjugated to the Ig constant region or to the fragment of the Ig constant region with fucose content of about between 1%-15%.

“Normal fucose” or ‘normal fucose content” as used herein refers to the antigen binding domain that bind CD79b conjugated to the Ig constant region or to the fragment of the Ig constant region with fucose content of about over 50%, typically about over 80% or over 85%.

Anti-Idiotypic Antibodies

Anti-idiotypic antibodies are antibodies that specifically bind to the antigen binding domain that binds CD79b of the disclosure.

The disclosure also provides an anti-idiotypic antibody that specifically binds to the antigen binding domain that binds CD79b of the disclosure.

The disclosure also provides an anti-idiotypic antibody that specifically binds to the antigen binding domain that binds CD79b comprising VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In one embodiment, the anti-idiotypic antibody that specifically binds to the antigen binding domain that binds CD79b comprising the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

An anti-idiotypic (Id) antibody is an antibody which recognizes the antigenic determinants (e.g. the paratope or CDRs) of the antibody. The Id antibody may be antigen-blocking or non-blocking. The antigen-blocking Id may be used to detect the free antigen binding domain in a sample (e.g. the antigen binding domain that binds CD79b of the disclosure). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original antigen binding domain which induced the anti-Id. Thus, by using antibodies to the idiotypic determinants of the antigen binding domain, it is possible to identify other clones expressing antigen binding domains of identical specificity. Anti-Id antibodies may be varied (thereby producing anti-Id antibody variants) and/or derivatized by any suitable technique, such as those described elsewhere herein.

Immunoconjugates

The antigen binding domains that bind CD79b of the disclosure, the proteins comprising the antigen binding domains that bind CD79b or the multispecific proteins that comprise the antigen binding domains that bind CD79b (collectively referred herein as to CD79b binding proteins) may be conjugated to a heterologous molecule.

In some embodiments, the heterologous molecule is a detectable label or a cytotoxic agent.

The invention also provides an antigen binding domain that binds CD79b conjugated to a detectable label.

The invention also provides a protein comprising an antigen binding domain that binds CD79b conjugated to a detectable label.

The invention also provides a multispecific protein comprising an antigen binding domain that binds CD79b conjugated to a detectable label.

The invention also provides an antigen binding domain that binds CD79b conjugated to a cytotoxic agent.

The invention also provides a protein comprising an antigen binding domain that binds CD79b conjugated to a cytotoxic agent.

The invention also provides a multispecific protein comprising an antigen binding domain that binds CD79b conjugated to a cytotoxic agent.

CD79b binding proteins of the disclosure may be used to direct therapeutics to CD79b expressing cells.

In some embodiments, the detectable label is also a cytotoxic agent.

The CD79b binding proteins of the disclosure conjugated to a detectable label may be used to evaluate expression of CD79b on a variety of samples.

Detectable label includes compositions that when conjugated to the CD79b binding proteins of the disclosure renders the latter detectable, via spectroscopic, photochemical, biochemical, immunochemical, or chemical means.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e. iodine) to 83 (i.e. bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu+, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The antigen binding domain that binds CD79b conjugated to a detectable label may be used as an imaging agent.

The protein comprising an antigen binding domain that binds CD79b conjugated to a detectable label may be used as an imaging agent.

The multispecific protein comprising an antigen binding domain that binds CD79b conjugated to a detectable label may be used as an imaging agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y, and ¹⁸⁶Re.

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the antibody of the invention through the N (amino) terminus or the C (carboxyl) terminus of the peptidic drug moiety (WO02/088172), or via any cysteine engineered into the antibody.

The CD79b binding proteins of the disclosure may be conjugated to a detectable label using known methods.

In some embodiments, the detectable label is complexed with a chelating agent.

In some embodiments, the detectable label is conjugated to the CD79b binding proteins of the disclosure via a linker.

The detectable label or the cytotoxic moiety may be linked directly, or indirectly, to the CD79b binding proteins of the disclosure using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiments, the CD79b binding proteins of the disclosure is removed from the blood via renal clearance.

Kits

The invention also provides a kit comprising the antigen binding domain that binds CD79b.

The invention also provides a kit comprising the protein comprising an antigen binding domain that binds CD79b.

The invention also provides a kit comprising the multispecific protein comprising an antigen binding domain that binds CD79b.

The kit may be used for therapeutic uses and as diagnostic kits.

The kit may be used to detect the presence of CD79b in a sample.

In some embodiments, the kit comprises the CD79b binding protein of the disclosure and reagents for detecting the CD79b binding protein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antigen binding domain that binds CD79b in a container and instructions for use of the kit.

In some embodiments, the kit comprises the protein comprising an antigen binding domain that binds CD79b in a container and instructions for use of the kit.

In some embodiments, the kit comprises the multispecific protein comprising an antigen binding domain that binds CD79b in a container and instructions for use of the kit.

In some embodiments, the antigen binding domain that binds CD79b in the kit is labeled.

In some embodiments, the protein comprising an antigen binding domain that binds CD79b in the kit is labeled.

In some embodiments, the multispecific protein comprising an antigen binding domain that binds CD79b in the kit is labeled.

In some embodiments, the kit comprises the antigen binding domain that binds CD79b comprising a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In some embodiments, the kit comprises the antigen binding domain that binds CD79b comprising a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

Polynucleotides, Host Cells and Vectors

The disclosure also provides an isolated polynucleotide encoding any of the CD79b binding proteins of the disclosure. The CD79b binding protein includes the antigen binding domains that bind CD79b, the proteins comprising the antigen binding domains that bind CD79b, the multispecific proteins that comprise the antigen binding domains that bind CD79b and the chimeric antigen receptors (CAR) comprising the antigen binding domains that bind CD79b of the disclosure.

The disclosure also provides an isolated polynucleotide encoding any of CD79b binding proteins or fragments thereof.

In one embodiment, the isolated polynucleotide encodes a CD79b binding protein comprising a heavy chain complementarity determining region (HCDR) 1 of SEQ ID NOs: 1, 11, 21, 31, 41, 51, 61, 71, 81, 91, 101, 111, 121, or 131. In one embodiment, the isolated polynucleotide encodes a CD79b binding protein comprising a HCDR2 of SEQ ID NOs: 72, 12, 22, 32, 42, 52, 62, 72, 82, 92, 102, 112, 122, or 132. In one embodiment, the isolated polynucleotide encodes CD79b binding protein comprising a HCDR3 of SEQ ID NOs: 3, 13, 23, 33, 43, 53, 63, 73, 83, 93, 103, 113, 123, or 133.

In one embodiment, the isolated polynucleotide encodes a CD79b binding protein comprising a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In one embodiment, the isolated polynucleotide encodes CD79b binding protein comprising a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In one embodiment, the isolated polynucleotide encodes CD79b binding protein comprising a light chain complementarity determining region (LCDR) 1 of SEQ ID NOs: 4, 14, 24, 34, 44, 54, 64, 74, 84, 94, 104, 114, 124, or 134. In one embodiment, the isolated polynucleotide encodes CD79b binding protein comprising a LCDR2 of SEQ ID NOs: 5, 15, 25, 35, 45, 55, 65, 75, 85, 95, 105, 115, 125, or 135. In one embodiment, the isolated polynucleotide encodes CD79b binding protein comprising a LCDR3 of SEQ ID NOs: 6, 16, 26, 36, 46, 56, 66, 76, 86, 96, 106, 116, 126, or 136.

In one embodiment, the isolated polynucleotide encodes CD79b binding protein comprising a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In one embodiment, the isolated polynucleotide encodes CD79b binding protein comprising a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In one embodiment, the isolated polynucleotide encodes CD79b binding protein comprising a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136. In one embodiment, the isolated polynucleotide encodes CD79b binding protein comprising a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In one embodiment, the isolated polynucleotide encodes CD79b binding protein comprising a VH of SEQ ID NOs: 7, 17, 27, 37, 47, 57, 67, 77, 87, 97, 107, 117, 127, or 137.

In one embodiment, the isolated polynucleotide encodes CD79b binding protein comprising a VL of SEQ ID NOs: 8, 18, 28, 38, 48, 58, 68, 78, 88, 98, 108, 118, 128, or 138.

In one embodiment, the isolated polynucleotide encodes CD79b binding protein comprising a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138 In one embodiment, the isolated polynucleotide encodes CD79b binding protein comprising a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In one embodiment, the isolated polynucleotide encodes CD79b binding protein comprising a heavy chain (HC) of SEQ ID NOs: 9, 19, 29, 39, 49, 59, 69, 79, 89, 99, 109, 119, 129, or 139.

In one embodiment, the isolated polynucleotide encodes CD79b binding protein comprising a light chain (LC) of SEQ ID NOs: 10, 20, 30, 40, 50, 60, 70, 80, 90, 100, 110, 120, 130, or 140.

In one embodiment, the isolated polynucleotide encodes CD79b binding protein comprising the HC of SEQ ID NOs: 9, 19, 29, 39, 49, 59, 69, 79, 89, 99, 109, 119, 129, or 139 and the LC of SEQ ID NOs: 10, 20, 30, 40, 50, 60, 70, 80, 90, 100, 110, 120, 130, or 140.

In one embodiment, the isolated polynucleotide encodes CD79b binding protein comprising the HC and LC of:
SEQ ID NOs: 9 and 10, respectively;
SEQ ID NOs: 19 and 20, respectively;
SEQ ID NOs: 29 and 30, respectively;
SEQ ID NOs: 39 and 40, respectively;
SEQ ID NOs: 49 and 50, respectively;
SEQ ID NOs: 59 and 60, respectively;
SEQ ID NOs: 69 and 70, respectively;
SEQ ID NOs: 79 and 80, respectively;
SEQ ID NOs: 89 and 90, respectively;
SEQ ID NOs: 99 and 100, respectively;
SEQ ID NOs: 109 and 110, respectively;
SEQ ID NOs: 119 and 120, respectively;
SEQ ID NOs: 129 and 130, respectively; or
SEQ ID NOs: 139 and 140, respectively.

Some embodiments of the disclosure also provide an isolated or purified nucleic acid comprising a polynucleotide which is complementary to the polynucleotides encoding the CD79b binding proteins of the disclosure or polynucleotides which hybridize under stringent conditions to the polynucleotides encoding the CD79b binding proteins of the disclosure.

The polynucleotides which hybridize under stringent conditions may hybridize under high stringency conditions. By “high stringency conditions” is meant that the polynucleotide specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

The polynucleotide sequences of the disclosure may be operably linked to one or more regulatory elements, such as a promoter or enhancer, that allow expression of the nucleotide sequence in the intended host cell. The polynucleotide may be a cDNA. The promoter bay be a strong, weak, tissue-specific, inducible or developmental-specific promoter. Exemplary promoters that may be used are hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. Inducible promoters such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like may also be used.

The invention also provides a vector comprising the polynucleotide of the invention. The disclosure also provide an expression vector comprising the polynucleotide of the invention. Such vectors may be plasmid vectors, viral vectors, vectors for baculovirus expression, transposon based vectors or any other vector suitable for introduction of the synthetic polynucleotide of the invention into a given organism or genetic background by any means. Polynucleotides encoding the CD79b binding proteins of the disclosure may be operably linked to control sequences in the expression vector(s) that ensure the expression of the CD79b binding proteins. Such regulatory elements may include a transcriptional promoter, sequences encoding suitable mRNA ribosomal binding sites, and sequences that control the termination of transcription and translation. Expression vectors may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The expression vectors can comprise naturally-occurring or non-naturally-occurring internucleotide linkages, or both types of linkages. The non-naturally occurring or altered nucleotides or internucleotide linkages do not hinder the transcription or replication of the vector.

Once the vector has been incorporated into the appropriate host, the host is maintained under conditions suitable for high level expression of the CD79b binding proteins of the disclosure encoded by the incorporated polynucleotides. The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

Vectors of the disclosure may also contain one or more Internal Ribosome Entry Site(s) (IRES). Inclusion of an IRES sequence into fusion vectors may be beneficial for enhancing expression of some proteins. In some embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent.

Suicide genes are known in the art and include, for example, the Herpes Simplex Virus (HSV) thymidine kinase (TK) gene, cytosine deaminase, purine nucleoside phosphorylase, and nitroreductase.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol x resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Exemplary vectors that may be used are Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Exemplary plant expression vectors include pBI01, pBI01.2, pBI121, pBI101.3, and pBIN19 (Clontech). Exemplary animal expression vectors include pEUK-Cl, pMAM, and pMAMneo (Clontech). The expression vector may be a viral vector, e.g., a retroviral vector, e.g., a gamma retroviral vector.

In some embodiments, the vector comprises a polynucleotide encoding a heavy chain complementarity determining region (HCDR) 1 of SEQ ID NOs: 1, 11, 21, 31, 41, 51, 61, 71, 81, 91, 101, 111, 121, or 131.

In some embodiments, the vector comprises a polynucleotide encoding a HCDR2 of SEQ ID NOs: 2, 12, 22, 32, 42, 52, 62, 72, 82, 92, 102, 112, 122, or 132.

In some embodiments, the vector comprises a polynucleotide encoding a HCDR3 of SEQ ID NOs: 3, 13, 23, 33, 43, 53, 63, 73, 83, 93, 103, 113, 123, or 133.

In some embodiments, the vector comprises a polynucleotide encoding a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; and a HCDR3 of SEQ ID NOs: 3, 13, 23, 33, 43, 53, 63, 73, 83, 93, 103, 113, 123, or 133.

In some embodiments, the vector comprises a polynucleotide encoding a HCDR1, HCDR2 and HCDR3 of:
SEQ ID NOs: 1, 2, and 3, respectively;
SEQ ID NOs: 11, 12, and 13, respectively;
SEQ ID NOs: 21, 22, and 23, respectively;
SEQ ID NOs: 31, 32, and 33, respectively
SEQ ID NOs: 41, 42, and 43, respectively
SEQ ID NOs: 51, 52, and 53, respectively
SEQ ID NOs: 61, 62, and 63, respectively
SEQ ID NOs: 71, 72, and 73, respectively
SEQ ID NOs: 81, 82, and 83, respectively
SEQ ID NOs: 91, 92, and 93, respectively
SEQ ID NOs: 101, 102, and 103, respectively
SEQ ID NOs: 111, 112, and 113, respectively
SEQ ID NOs: 121, 122, and 123, respectively; or
SEQ ID NOs: 131, 132, and 133, respectively.

In some embodiments, the vector comprises a polynucleotide encoding a light chain complementarity determining region (LCDR) 1 of SEQ ID NOs: 4, 14, 24, 34, 44, 54, 64, 74, 84, 94, 104, 114, 124, or 134.

In some embodiments, the vector comprises a polynucleotide encoding a LCDR2 of SEQ ID NOs: 5, 15, 25, 35, 45, 55, 65, 75, 85, 95, 105, 115, 125, or 135.

In some embodiments, the vector comprises a polynucleotide encoding a LCDR3 of SEQ ID NOs: 6, 16, 26, 36, 46, 56, 66, 76, 86, 96, 106, 116, 126, or 136.

In some embodiments, the vector comprises a polynucleotide encoding a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the vector comprises a polynucleotide encoding a LCDR, LCDR2 and LCDR3 of:
SEQ ID NOs: 4, 5, and 6, respectively;
SEQ ID NOs: 14, 15, and 16, respectively;
SEQ ID NOs: 24, 25, and 26, respectively;
SEQ ID NOs: 34, 35, and 36, respectively;
SEQ ID NOs: 44, 45, and 46, respectively;
SEQ ID NOs: 54, 55, and 56, respectively;
SEQ ID NOs: 64, 65, and 66, respectively;
SEQ ID NOs: 74, 75, and 76, respectively;
SEQ ID NOs: 84, 85, and 86, respectively;
SEQ ID NOs: 94, 95, and 96, respectively;
SEQ ID NOs: 104, 105, and 106, respectively;
SEQ ID NOs: 114, 115, and 116, respectively;
SEQ ID NOs: 124, 125, and 126, respectively; or
SEQ ID NOs: 134, 135, and 136, respectively.

In some embodiments, the vector comprises a polynucleotide encoding HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136.

In some embodiments, the vector comprises a polynucleotide encoding a HCDR1, HCDR2, HCDR3, LCDR1, LCDR2 and LCDR3 of:
SEQ ID NOs: 1, 2, 3, 4, 5, and 6, respectively;
SEQ ID NOs: 11, 12, 13, 14, 15, and 16, respectively;
SEQ ID NOs: 21, 22, 23, 24, 25, and 26, respectively;
SEQ ID NOs: 31, 32, 33, 34, 35, and 36, respectively;
SEQ ID NOs: 41, 42, 43, 44, 45, and 46, respectively;
SEQ ID NOs: 51, 52, 53, 54, 55, and 56, respectively;
SEQ ID NOs: 61, 62, 63, 64, 65, and 66, respectively;
SEQ ID NOs: 71, 72, 73, 74, 75, and 76, respectively;
SEQ ID NOs: 81, 82, 83, 84, 85, and 86, respectively;
SEQ ID NOs: 91, 92, 93, 94, 95, and 96, respectively;
SEQ ID NOs: 101, 102, 103, 104, 105, and 106, respectively;
SEQ ID NOs: 111, 112, 113, 114, 115, and 116, respectively;
SEQ ID NOs: 121, 122, 123, 124, 125, and 126, respectively; or
SEQ ID NOs: 131, 132, 133, 134, 135, and 136, respectively.

In some embodiments, the vector comprises a polynucleotide encoding the VH of SEQ ID NOs: 7, 17, 27, 37, 47, 57, 67, 77, 87, 97, 107, 117, 127, or 137.

In some embodiments, the vector comprises a polynucleotide encoding the VL of SEQ ID NOs: 8, 18, 28, 38, 48, 58, 68, 78, 88, 98, 108, 118, 128, or 138.

In some embodiments, the vector comprises a polynucleotide encoding the VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138.

In some embodiments, the vector comprises a polynucleotide encoding the VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

In one embodiment, the vector comprises a polynucleotide encoding the heavy chain (HC) of SEQ ID NOs: 9, 19, 29, 39, 49, 59, 69, 79, 89, 99, 109, 119, 129, or 139.

In one embodiment, the vector comprises a polynucleotide encoding the light chain (LC) of SEQ ID NOs: 10, 20, 30, 40, 50, 60, 70, 80, 90, 100, 110, 120, 130, or 140.

In one embodiment, the vector comprises a polynucleotide encoding the HC of SEQ ID NOs: 9, 19, 29, 39, 49, 59, 69, 79, 89, 99, 109, 119, 129, or 139 and the LC of SEQ ID NOs: 10, 20, 30, 40, 50, 60, 70, 80, 90, 100, 110, 120, 130, or 140.

In one embodiment, the vector comprises a polynucleotide encoding the HC and LC of:
SEQ ID NOs: 9 and 10, respectively;
SEQ ID NOs: 19 and 20, respectively;
SEQ ID NOs: 29 and 30, respectively;
SEQ ID NOs: 39 and 40, respectively;
SEQ ID NOs: 49 and 50, respectively;
SEQ ID NOs: 59 and 60, respectively;
SEQ ID NOs: 69 and 70, respectively;
SEQ ID NOs: 79 and 80, respectively;
SEQ ID NOs: 89 and 90, respectively;
SEQ ID NOs: 99 and 100, respectively;
SEQ ID NOs: 109 and 110, respectively;
SEQ ID NOs: 119 and 120, respectively;
SEQ ID NOs: 129 and 130, respectively; or
SEQ ID NOs: 139 and 140, respectively.

Embodiments of the invention further provide host cells comprising any of the recombinant expression vectors described herein. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, polypeptide, or protein, the host cell may be a mammalian cell. The host cell may be a human cell. While the host cell can be of any cell type, can originate from any type of tissue, and can be of any developmental stage, the host cell may be a peripheral blood lymphocyte (PBL). The host cell may be a T cell.

Also provided are a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, an erythrocyte, a neutrophil, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

The disclosure also provides a method of producing the CD79b binding protein of the disclosure comprising culturing the host cell of the disclosure in conditions that the CD79b binding protein is expressed, and recovering the CD79b binding protein produced by the host cell. Methods of making proteins and purifying them are known. Once synthesized (either chemically or recombinantly), the CD79b binding proteins may be purified according to standard procedures, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

The polynucleotides encoding the CD79b binding proteins of the disclosure may be incorporated into vectors using standard molecular biology methods. Host cell transformation, culture, antibody expression and purification are done using well known methods.

Modified nucleotides may be used to generate the polynucleotides of the disclosure. Exemplary modified nucleotides are 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N⁶-isopentenyladenine, uracil-5-oxyacetic acid (v), wybutoxosine, pseudouracil, queuosine, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

Pharmaceutical Compositions/Administration

The disclosure also provides a pharmaceutical composition comprising the CD79b binding protein of the disclosure and a pharmaceutically acceptable carrier.

The disclosure also provides a pharmaceutical composition comprising the antigen binding domain that binds CD79b of the disclosure and a pharmaceutically acceptable carrier.

The disclosure also provides a pharmaceutical composition comprising the protein comprising the antigen binding domain that binds CD79b of the disclosure and a pharmaceutically acceptable carrier.

The disclosure also provides a pharmaceutical composition comprising the multispecific protein comprising the antigen binding domain that binds CD79b of the disclosure and a pharmaceutically acceptable carrier.

The disclosure also provides a pharmaceutical composition comprising the antigen binding domain that binds CD79b of the disclosure and a pharmaceutically acceptable carrier.

For therapeutic use, the CD79b binding protein of the disclosure may be prepared as pharmaceutical compositions containing an effective amount of the antibody as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the CD79-binding proteins of the invention in such pharmaceutical formulation may vary, from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, P A 2006, Part 5, Pharmaceutical Manufacturing pp 691-1092, See especially pp. 958-989.

The mode of administration of the CD79b binding protein of the disclosure may be any suitable route such as parenteral administration, e.g., intradermal, intramuscular, intraperitoneal, intravenous or subcutaneous, transmucosal (oral, intranasal, intravaginal, rectal) or other means appreciated by the skilled artisan, as well known in the art.

The CD79b binding protein of the disclosure of the invention may also be administered prophylactically in order to reduce the risk of developing a disease such as cancer.

Thus, a pharmaceutical composition of the invention for intramuscular injection may be prepared to contain 1 ml sterile buffered water, and between about 1 ng to about 100 mg/kg, e.g. about 50 ng to about 30 mg/kg or more preferably, about 5 mg to about 25 mg/kg, of the CD79b binding protein of the disclosure of the invention.

In embodiments of the present disclosure, the CD79b binding protein-expressing cells may be provided in compositions, e.g., suitable pharmaceutical composition(s) comprising the CD79b binding protein-expressing cells and a pharmaceutically acceptable carrier. In one aspect, the present disclosure provides pharmaceutical compositions comprising an effective amount of a lymphocyte expressing one or more of the CD79b binding proteins described and a pharmaceutically acceptable excipient. Pharmaceutical compositions of the present disclosure may comprise a CD79b binding protein-expressing cell, e.g., a plurality of CD79b binding protein-expressing cells, as described herein, in combination with one or more pharmaceutically or physiologically acceptable carriers, excipients or diluents. A pharmaceutically acceptable carrier can be an ingredient in a pharmaceutical composition, other than an active ingredient, which is nontoxic to the subject.

A pharmaceutically acceptable carrier can include a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Pharmaceutical compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Pharmaceutical compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

Methods of Detecting CD79B

The disclosure also provides a method of detecting CD79b in a sample, comprising obtaining the sample, contacting the sample with the antigen binding domain that binds CD79b of the disclosure and detecting the bound CD79b in the sample.

In some embodiments, the sample may be derived from urine, blood, serum, plasma, saliva, ascites, circulating cells, synovial fluid, circulating cells, cells that are not tissue associated (i.e., free cells), tissues (e.g., surgically resected tissue, biopsies, including fine needle aspiration), histological preparations, and the like.

The antigen binding domain that binds CD79b of the disclosure may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antigen binding domain that binds CD79b of the disclosure may be used in a variety of assays to detect CD79b in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

Methods of Treatment and Uses

The antigen binding domain that binds CD79b of the disclosure may be administered to a subject in need thereof to manage, treat, prevent, or ameliorate an autoimmune disease or disorder or one or more symptoms thereof.

The disclosure also provides methods comprising administering a therapeutically effective amount of the antigen binding domain that binds CD79b of the disclosure to a subject having an autoimmune disease.

The disclosure also provides methods comprising administering a therapeutically effective amount of the protein comprising the antigen binding domain that binds CD79b of the disclosure to a subject having an autoimmune disease.

The disclosure also provides a method comprising administering a therapeutically effective amount of the multispecific protein comprising the antigen binding domain that binds CD79b of the disclosure to a subject having an autoimmune disease.

The disclosure also provides a method comprising administering a therapeutically effective amount of the antigen binding domain that binds CD79b of the disclosure to a subject having an autoimmune disease.

The disclosure also provides a method comprising administering a therapeutically effective amount of the immunoconjugate comprising the antigen binding domain that binds CD79b of the disclosure to a subject having an autoimmune disease.

The disclosure also provides a method comprising administering a therapeutically effective amount of the pharmaceutical composition comprising the antigen binding domain that binds CD79b of the disclosure to a subject having an autoimmune disease.

The disclosure also provides methods of treating an autoimmune disease in a subject comprising administering a therapeutically effective amount of the antigen binding domain that binds CD79b of the disclosure to the subject in need thereof for a time sufficient to treat the autoimmune disease.

The disclosure also provides methods of treating an autoimmune disease in a subject comprising administering a therapeutically effective amount of the protein comprising the antigen binding domain that binds CD79b of the disclosure to the subject for a time sufficient to treat the autoimmune disease.

The disclosure also provides a method of treating an autoimmune disease in a subject, comprising administering a therapeutically effective amount of the multispecific protein comprising the antigen biding domain that binds CD79b of the disclosure to the subject for a time sufficient to treat the autoimmune disease.

The disclosure also provides a method of treating an autoimmune disease in a subject, comprising administering a therapeutically effective amount of the antigen biding domain that binds CD79b of the disclosure to the subject for a time sufficient to treat the autoimmune disease.

The disclosure also provides a method of treating an autoimmune disease in a subject, comprising administering a therapeutically effective amount of the immunoconjugate comprising the antigen binding domain that binds CD79b of the disclosure to the subject for a time sufficient to treat the autoimmune disease.

The disclosure also provides a method of treating an autoimmune disease in a subject, comprising administering a therapeutically effective amount of the pharmaceutical composition comprising the antigen binding domain that binds CD79b of the disclosure to the subject for a time sufficient to treat the autoimmune disease.

In one embodiment, the autoimmune disease is associated with or characterized by dysregulation of B cells, autoreactive B cells, or the presence of autoantibodies. Examples of autoimmune diseases include, but are not limited to, Systemic lupus erythematosus (SLE), Sjögren's syndrome (SjS), Rheumatoid arthritis, Autoimmune myopathies, Type I diabetes, Addison disease, Pernicious anemia, Autoimmune hepatitis, Primary biliary cholangitis (PBC), Autoimmune pancreatitis, Celiac disease, Focal segmental glomerulosclerosis, Primary membranous nephropathy, Ovarian insufficiency, Autoimmune orchitis, Dry eye disease, Idiopathic interstitial pneumonias, Thyroid disease (eg Grave's), Systemic sclerosis (Scleroderma), Myasthenic syndromes, Autoimmune encephalitis, Bullous skin diseases, TTP, ITP, AIHA, Anca vasculitis, Myocarditis/dilatory CM, NMOSD, Maternal-fetal alloimmunity, Maternal-fetal autoimmunity, Anti-cardiolipin/antiphospholipid syndrome, Hypergammaglobulinemia, Transplant-associated ID, Multifocal motor neuropathy. The disclosure also provides methods of preventing an autoimmune disease in a subject comprising administering a therapeutically effective amount of the antigen binding domain that binds CD79b of the disclosure to the subject in need thereof for a time sufficient to prevent the autoimmune disease. IN certain embodiments, preventing comprises treating an asymptomatic subject. In certain embodiments, preventing comprises preventing the onset of autoimmune disease symptoms in a subject.

The disclosure also provides methods of preventing an autoimmune disease in a subject comprising administering a therapeutically effective amount of the protein comprising the antigen binding domain that binds CD79b of the disclosure to the subject for a time sufficient to treat the autoimmune disease.

The disclosure also provides a method of preventing an autoimmune disease in a subject, comprising administering a therapeutically effective amount of the multispecific protein comprising the antigen binding domain that binds CD79b of the disclosure to the subject for a time sufficient to prevent the autoimmune disease.

The disclosure also provides a method of preventing an autoimmune disease in a subject, comprising administering a therapeutically effective amount of the antigen binding domain that binds CD79b of the disclosure to the subject for a time sufficient to prevent the autoimmune disease.

The disclosure also provides a method of preventing an autoimmune disease in a subject, comprising administering a therapeutically effective amount of the immunoconjugate comprising the antigen binding domain that binds CD79b of the disclosure to the subject for a time sufficient to prevent the autoimmune disease.

The disclosure also provides a method of preventing an autoimmune disease in a subject, comprising administering a therapeutically effective amount of the pharmaceutical composition comprising the antigen binding domain that binds CD79b of the disclosure to the subject for a time sufficient to prevent the autoimmune disease.

In one embodiment, the method of preventing an autoimmune disease in a subject further comprises detecting autoantibodies in the subject.

The disclosure also provides methods of modulating B cell activation in a subject comprising administering a therapeutically effective amount of the antigen binding domain that binds CD79b of the disclosure to the subject in need thereof for a time sufficient to modulate B cell activation.

The disclosure also provides methods of modulating B cell activation in a subject comprising administering a therapeutically effective amount of the protein comprising the antigen binding domain that binds CD79b of the disclosure to the subject for a time sufficient to modulate B cell activation.

The disclosure also provides a method of modulating B cell activation in a subject, comprising administering a therapeutically effective amount of the multi-specific protein comprising the antigen binding domain that binds CD79b of the disclosure to the subject for a time sufficient to modulate B cell activation.

The disclosure also provides a method of modulating B cell activation in a subject, comprising administering a therapeutically effective amount of the antigen binding domain that binds CD79b of the disclosure to the subject for a time sufficient to modulate B cell activation.

The disclosure also provides a method of modulating B cell activation in a subject, comprising administering a therapeutically effective amount of the immunoconjugate comprising the antigen binding domain that binds CD79b of the disclosure to the subject for a time sufficient to modulate B cell activation.

The disclosure also provides a method of modulating B cell activation in a subject, comprising administering a therapeutically effective amount of the pharmaceutical composition comprising the antigen binding domain that binds CD79b of the disclosure to the subject for a time sufficient to modulate B cell activation.

The disclosure also provides methods of inhibiting aberrant B cell activation in a subject comprising administering a therapeutically effective amount of the antigen binding domain that binds CD79b of the disclosure to the subject in need thereof for a time sufficient to inhibit aberrant B cell activation.

The disclosure also provides methods of inhibiting aberrant B cell activation in a subject comprising administering a therapeutically effective amount of the protein comprising the antigen binding domain that binds CD79b of the disclosure to the subject for a time sufficient to inhibit aberrant B cell activation.

The disclosure also provides a method of inhibiting aberrant B cell activation in a subject, comprising administering a therapeutically effective amount of the multispecific protein comprising the antigen binding domain that binds CD79b of the disclosure to the subject for a time sufficient to inhibit aberrant B cell activation.

The disclosure also provides a method of inhibiting aberrant B cell activation in a subject, comprising administering a therapeutically effective amount of the antigen binding domain that binds CD79b of the disclosure to the subject for a time sufficient to inhibit aberrant B cell activation.

The disclosure also provides a method of inhibiting aberrant B cell activation in a subject, comprising administering a therapeutically effective amount of the immunoconjugate comprising the antigen binding domain that binds CD79b of the disclosure to the subject for a time sufficient to inhibit aberrant B cell activation.

The disclosure also provides a method of inhibiting aberrant B cell activation in a subject, comprising administering a therapeutically effective amount of the pharmaceutical composition comprising the antigen binding domain that binds CD79b of the disclosure to the subject for a time sufficient to inhibit aberrant B cell activation.

The disclosure also provides methods of treating an autoimmune disease in a subject comprising administering a therapeutically effective amount of a composition comprising an antigen binding domain that binds CD79b of the disclosure to the subject in need thereof for a time sufficient to treat the autoimmune disease, wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136. In one embodiment, the antigen binding domain that binds CD79b comprises a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In one embodiment, the antigen binding domain that binds CD79b comprises a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

The disclosure also provides methods of preventing an autoimmune disease in a subject comprising administering a therapeutically effective amount of a composition comprising an antigen binding domain that binds CD79b of the disclosure to the subject in need thereof for a time sufficient to prevent the autoimmune disease, wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136. In one embodiment, the antigen binding domain that binds CD79b comprises VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In one embodiment, the antigen binding domain that binds CD79b comprises a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

The disclosure also provides methods of modulating B cell activation in a subject comprising administering a therapeutically effective amount of a composition comprising an antigen binding domain that binds CD79b of the disclosure to the subject in need thereof for a time sufficient to modulate B cell activation, wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136. In one embodiment, the antigen binding domain that binds CD79b comprises a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In one embodiment, the antigen binding domain that binds CD79b comprises a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

The disclosure also provides methods of inhibiting aberrant B cell activation in a subject comprising administering a therapeutically effective amount of a composition comprising an antigen binding domain that binds CD79b of the disclosure to the subject in need thereof for a time sufficient to inhibit aberrant B cell activation, wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136. In one embodiment, the antigen binding domain that binds CD79b comprises a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In one embodiment, the antigen binding domain that binds CD79b comprises a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively.

The disclosure also provides a method comprising administering a composition comprising an antigen binding domain that binds CD79b of the disclosure to a subject, wherein the antigen binding domain that binds CD79b comprises a HCDR1 of SEQ ID NOs: 1, 11, 21, 31, 41, 51, 61, 71, 81, 91, 101, 111, 121, or 131; a HCDR2 of SEQ ID NOs: 2, 12, 22, 32, 42, 52, 62, 72, 82, 92, 102, 112, 122, or 132; a HCDR3 of SEQ ID NOs: 3, 13, 23, 33, 43, 53, 63, 73, 83, 93, 103, 113, 123, or 133; a LDCR1 of SEQ ID NOs: 4, 14, 24, 34, 44, 54, 64, 74, 84, 94, 104, 114, 124, or 134; a LCDR2 of SEQ ID NOs: 5, 15, 25, 35, 45, 55, 65, 75, 85, 95, 105, 115, 125, or 135; and a LCDR3 of SEQ ID NOs: 6, 16, 26, 36, 46, 56, 66, 76, 86, 96, 106, 116, 126, or 136. In one embodiment, the antigen binding domain that binds CD79b comprises a VH of SEQ ID NOs:7, 17, 27, 37, 47, 57, 67, 77, 87, 97, 107, 117, 127, or 137; and the VL of SEQ ID NOs: 8, 18, 28, 38, 48, 58, 68, 78, 88, 98, 108, 118, 128, or 138. In one embodiment, the antigen binding domain that binds CD79b comprises a VH and VL of:
SEQ ID NOs: 7 and 8, respectively;
SEQ ID NOs: 17 and 18, respectively;
SEQ ID NOs: 27 and 28, respectively;
SEQ ID NOs: 37 and 38, respectively;
SEQ ID NOs: 47 and 48, respectively;
SEQ ID NOs: 57 and 58, respectively;
SEQ ID NOs: 67 and 68, respectively;
SEQ ID NOs: 77 and 78, respectively;
SEQ ID NOs: 87 and 88, respectively;
SEQ ID NOs: 97 and 98, respectively;
SEQ ID NOs: 107 and 108, respectively;
SEQ ID NOs: 117 and 118, respectively;
SEQ ID NOs: 127 and 128, respectively; or
SEQ ID NOs: 137 and 138, respectively. In one embodiment, the subject has an autoimmune disease or disorder.

When a therapeutically effective amount is indicated, the precise amount of the CD79-binding proteins of the present disclosure to be administered can be determined by a physician with consideration of individual differences in age, weight, and condition of the subject.

Delivery systems useful in the context of the CD79-binding proteins of the invention may include time-released, delayed release, and sustained release delivery systems such that the delivery of the CD79-binding protein compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the invention.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation. The administration of the CD79-binding proteins and compositions may be carried out in any manner, e.g., by parenteral or nonparenteral administration, including by aerosol inhalation, injection, infusions, ingestion, transfusion, implantation or transplantation. For example, the CD79-binding proteins and compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a subject by intradermal or subcutaneous injection. The compositions of CD79-binding proteins may be injected, for instance, directly into a tumor, lymph node, tissue, organ, or site of infection.

In one embodiment, administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

Combination Therapies

The CD79b binding proteins of the disclosure may be administered in combination with at least one additional therapeutics.

The CD79b binding proteins of the disclosure may also be administered in combination with one or more other therapies. In some embodiments, the CD79b binding proteins of the disclosure may be administered in combination with one or more other therapies useful for the prevention, management, treatment or amelioration of an autoimmune disease or disorder or one or more symptoms thereof to a subject in need thereof to prevent, manage, treat or ameliorate an autoimmune disease or disorder or one or more symptoms thereof.

In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one embodiment, other therapeutic agents such as factors may be administered before, after, or at the same time (simultaneous with) as the CD79b binding proteins.

The CD79b binding proteins such as CAR-expressing cell described herein and the at least one additional therapeutic agent can be administered simultaneously, in the same or in separate compositions, or sequentially. For sequential administration, the CD79b binding proteins described herein can be administered first, and the additional agent can be administered second, or the order of administration can be reversed.

In one embodiment, the subject can be administered an agent which enhances the activity of a CD79b binding protein. For example, in one embodiment, the agent can be an agent which inhibits an inhibitory molecule.

The following examples are provided to further describe some of the embodiments disclosed herein. The examples are intended to illustrate, not to limit, the disclosed embodiments.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Generation of Soluble CD79B ECD Protein

The extracellular domain (ECD) of human (h) CD79B isoform 1 was obtained commercially (R&D Cas 9687-CD Lot: TLS021805A) and used for immunization efforts. The extracellular domain (ECD) of human (h) CD79B isoform 1, named CD9W7 (SEQ ID NO:178), and hCD79B isoform 2, named CD9W8 (SEQ ID NO: 179) were expressed and purified for use in binding and affinity measurements. The cDNA encoding each protein was prepared using gene synthesis techniques (U.S. Pat. Nos. 6,670,127; 6,521,427) and the plasmids for expression were prepared using standard molecular biology techniques. Furthermore, each ECD protein had 6×-His tags at the C-terminus for ease of purification.

CD79b long isoform (CD9W7) Note: bolded segment is signal sequence
(SEQ ID NO: 178)
MARSALLILALLLLGLFSPGAWGARSEDRYRNPKGSACSRIWQSPRFIARKRGFTVKMHC
YMNSASGNVSWLWKQEMDENPQQLKLEKGRMEESQNESLATLTIQGIRFEDNGIYFCQQKC
NNTSEVYQGCGTELRVMGFSTLAQLKQRNTLKDGSLNDIFEAQKIEWHEHHHHHH
CD79b short isoform (CD9W8)
(SEQ ID NO: 179)
MARSALLILALLLLGLFSPGAWGARSEDRYRNPKGFSTLAQLKQRNTLKDGSLNDIFEAQKIE
WHEHHHHHH

Isolation of Human CD79B Monoclonal Antibody Expressing Hybridomas from Ablexis Mice

A human immunoglobulin transgenic mouse strain (Ablexis; AlivaMab, LLC.) was used to develop human CD79b monoclonal antibodies. The Ablexis mice contain a chimeric human/mouse IgH locus (comprising of 32 human V alleles, 27 human D alleles and 6 human J alleles in natural configuration linked to the mouse C_{H} locus) together with fully human IgL locus (comprising of 18 Vκ alleles and 5 Jκ alleles and/or 29 V_{λ} alleles and 7 J_{λ} alleles linked to appropriate mouse Cλ or Cκ). Accordingly, the mice contain an inactivated endogenous Ig locus, and in response to immunization, the introduced human heavy and light chain transgenes undergo class switching and somatic mutation to generate high affinity human IgG monoclonal antibodies. The preparation and use of Ablexis, and the genomic modifications carried by such mice, is described in Patent #20130167256 to L. Green et al. When immunized with recombinant human CD79b (rhCD79b), this transgenic mouse produces human IgG antibodies specific to human CD79b.

Two immunization schemes and recovery paths were performed. For Hybridoma Strategy 1 (Hyb:649)—the immunization strategy in Ablexis kappa mice consisted of RIMMS+IP injections rhCD79b (R&D Cas 9687-CD Lot: TLS021805A) in sigma adjuvant (Sigma, Catalog S6322) (days 0, 8, 13, and 20). On day 31, after sufficient titers were reached, mice were given a final boost of rhCD79b (R&D Cas 9687-CD Lot: TLS021805A)+anti-msCD40 (R&D cat #MAB440; lot: AHY181704A) 4 days prior to fusion. Spleens and mandibular, accessory mandibular, superficial parotid, proper axillary, accessory axillary, subiliac, sciatic, popliteal, gastric, pancreaticodoudenal, jejunal, and medial iliac lymph nodes were harvested and used to generate hybridomas. Sixty plates of hybridoma supernatants were screened by cell-based MSD to identify mAbs which exhibited binding to rhCD79b. After further confirmatory screenings, hybridoma supernatants from both screens that exhibited binding specific to human CD79b expressing SU-DHL-4 & SU-DHL-10 cells (CD79a/b expressing primary cell lines, AG000002269 & AG000002270, respectively) were sequenced, cloned and expressed in small scale. For Hybridoma Strategy 2 (Hyb:650)—the immunization strategy in Ablexis kappa mice consisted of a RIMMSIP injections of rhCD79b (R&D Cas 9687-CD Lot: TLS021805A) in CL413 (InvivoGen cat #vac-cl413-5) (days 42, 49, and 56) or Sigma (Sigma, Catalog S6322) (days 72, 79, 86, and 114). On day 129, after sufficient titers were reached, mice were given a final boost of rhCD79b (R&D Cas 9687-CD Lot: TLS021805A)+CL413 (InvivoGen cat #vac-cl413-5)+CD40 (R&D cat #MAB440; lot: AHY181704A) 7 days prior to sorting. Spleens and mandibular, accessory mandibular, superficial parotid, proper axillary, accessory axillary, subiliac, sciatic, popliteal, gastric, pancreaticodoudenal, jejunal, and medial iliac lymph nodes were harvested, and antigen-positive B cells were isolated by Fluorescence-activated cell sorting (FACS). Ten 384-well plates of sorted B cell supernatants were screened by cell-based MSD to identify mAbs with binding to human CD79B expressing SU-DHL-10 cells (CD79a/b expressing primary cell lines, AG000002270). Positive clones were sequenced, cloned and expressed in small scale.

Identification of Additional CD79B Binders

New CD79B binding arms were generated from a mouse immunization campaign. The most suitable mAbs were selected and triaged based on following criteria:

B cell line binding data (Table 2)

All binders with positive HC or LC Epivax scores were deprioritized

All binders with identical CDR on different framework were eliminated based on total Epivax score and total # of PTM motifs.

Three additional CD79B binders were identified with negative Epivax scores for HC & LC: CD9B1281, CD9B1315 and CD9B1256. CD9B1256 has a hCD79B-long binding affinity (SPR, nM) of 0.78 and a hCD79B-short binding affinity (SPR, nM) of 2.21.

TABLE 2
HC and LC sequences

| Binder# | VR# | Cell binding S/B Carnaval qAC50[M] |
|---|---|---|
| CD9B1281 | VR000056301 | 3.9E−08 |
| CD9B1315 | VR000056204 | 1.3E−09 |
| CD9B1256 | VR000056263 | 2.5E−10 |

TABLE 3
HC and LC sequences, HC and LC variable regions sequences, as well as CDRs
(ABM definitions)

| mAb ID | Region | Amino Acid Sequence | SEQ ID NO: |
|---|---|---|---|
| CD9B366 | HCDR1 | GFTFSNYWMS | 1 |
| | HCDR2 | NINQGGSEKY | 2 |
| | HCDR3 | DPILAALDY | 3 |
| | LCDR1 | RSSQSLVYSDGNTYLH | 4 |
| | LCDR2 | KVSNRDS | 5 |
| | LCDR3 | MQGTHWPRT | 6 |
| | VH | QVQLVESGGGLVQPGGSLRLSCSASGFTFSNYWMS | 7 |
| | | WVRQAPGKGLEWVANINQGGSEKYYVDSVEGRFT | |
| | | ISRDNAKNSLSLQMNGLRAEDTAVYYCARDPILAA | |
| | | LDYWGQGTLVTVSS | |
| | VL | DIVMTQSPLSLSVTLGQPASISCRSSQSLVYSDGNTY | 8 |
| | | LHWFHQRPGQSPRRLIYKVSNRDSGVPDRFSGTGSD | |
| | | TDFTLKISRVEAEDVGVYYCMQGTHWPRTFGQGTK | |
| | | VEI | |
| | HC | QVQLVESGGGLVQPGGSLRLSCSASGFTFSNYWMS | 9 |
| | | WVRQAPGKGLEWVANINQGGSEKYYVDSVEGRFT | |
| | | ISRDNAKNSLSLQMNGLRAEDTAVYYCARDPILAA | |
| | | LDYWGQGTLVTVSSASTKGPSVFPLAPSSKSTSGGT | |
| | | AALGCLVKDYFPEPVTVSWNSGALTSGVHTFPAVL | |
| | | QSSGLYSLSSVVTVPSSSLGTQTYICNVNHKPSNTK | |
| | | VDKKVEPKSCDKTHTCPPCPAPELLGGPSVFLFPPK | |
| | | PKDTLMISRTPEVTCVVVDVSHEDPEVKFNWYVDG | |
| | | VEVHNAKTKPREEQYNSTYRVVSVLTVLHQDWLN | |
| | | GKEYKCKVSNKALPAPIEKTISKAKGQPREPQVYTL | |
| | | PPSREEMTKNQVSLTCLVKGFYPSDIAVEWESNGQP | |
| | | ENNYKTTPPVLDSDGSFFLYSKLTVDKSRWQQGNV | |
| | | FSCSVMHEALHNHYTQKSLSLSPGK | |
| | LC | DIVMTQSPLSLSVTLGQPASISCRSSQSLVYSDGNTY | 10 |
| | | LHWFHQRPGQSPRRLIYKVSNRDSGVPDRFSGTGSD | |
| | | TDFTLKISRVEAEDVGVYYCMQGTHWPRTFGQGTK | |
| | | VEIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B366 | HCDR1 | GFTFSNYWMS | 11 |
| m1 | HCDR2 | NINQGGSEKY | 12 |
| | HCDR3 | EPILAALDY | 13 |
| | LCDR1 | RSSQSLVYSDGNTYLH | 14 |
| | LCDR2 | KVSNRDS | 15 |
| | LCDR3 | MQGTHWPRT | 16 |
| | VH | QVQLVESGGGLVQPGGSLRLSCSASGFTFSNYWMS | 17 |
| | | WVRQAPGKGLEWVANINQGGSEKYYVDSVEGRFT | |
| | | ISRDNAKNSLSLQMNGLRAEDTAVYYCAREPILAA | |
| | | LDYWGQGTLVTVSS | |
| | VL | DIVMTQSPLSLSVTLGQPASISCRSSQSLVYSDGNTY | 18 |
| | | LHWFHQRPGQSPRRLIYKVSNRDSGVPDRFSGTGSD | |
| | | TDFTLKISRVEAEDVGVYYCMQGTHWPRTFGQGTK | |
| | | VEI | |
| | HC | QVQLVESGGGLVQPGGSLRLSCSASGFTFSNYWMS | 19 |
| | | WVRQAPGKGLEWVANINQGGSEKYYVDSVEGRFT | |
| | | ISRDNAKNSLSLQMNGLRAEDTAVYYCAREPILAA | |
| | | LDYWGQGTLVTVSSASTKGPSVFPLAPSSKSTSGGT | |
| | | AALGCLVKDYFPEPVTVSWNSGALTSGVHTFPAVL | |
| | | QSSGLYSLSSVVTVPSSSLGTQTYICNVNHKPSNTK | |
| | | VDKKVEPKSCDKTHTCPPCPAPELLGGPSVFLFPPK | |
| | | PKDTLMISRTPEVTCVVVDVSHEDPEVKFNWYVDG | |
| | | VEVHNAKTKPREEQYNSTYRVVSVLTVLHQDWLN | |
| | | GKEYKCKVSNKALPAPIEKTISKAKGQPREPQVYTL | |
| | | PPSREEMTKNQVSLTCLVKGFYPSDIAVEWESNGQP | |
| | | ENNYKTTPPVLDSDGSFFLYSKLTVDKSRWQQGNV | |
| | | FSCSVMHEALHNHYTQKSLSLSPGK | |
| | LC | DIVMTQSPLSLSVTLGQPASISCRSSQSLVYSDGNTY | 20 |
| | | LHWFHQRPGQSPRRLIYKVSNRDSGVPDRFSGTGSD | |
| | | TDFTLKISRVEAEDVGVYYCMQGTHWPRTFGQGTK | |
| | | VEIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B376 | HCDR1 | GFTFNNYWMS | 21 |
| | HCDR2 | NIYRDGSEKY | 22 |
| | HCDR3 | DPSTGDLDY | 23 |
| | LCDR1 | RSSQSLVYHDGNTYLI | 24 |
| | LCDR2 | KVSNRDS | 25 |
| | LCDR3 | MQGSHWPRT | 26 |
| | VH | EVQLVESGGDLVQPGGSLRLSCIASGFTFNNYWMS | 27 |
| | | WVRQAPGKGLEWVANIYRDGSEKYYLDSVEGRFTI | |
| | | SRDNAKNSLYLQMNSLRLEDTAVYYCARDPSTGDL | |
| | | DYWGQGTPVTVSS | |
| | VL | DIQMTQSPLSLSVTLGQPASISCRSSQSLVYHDGNTY | 28 |
| | | LIWFQQRPGQSPRRLIYKVSNRDSGVPDRFSGSGSG | |
| | | TDFTLKISRVEAEDVGVYYCMQGSHWPRTFGPGTK | |
| | | VDIK | |
| | HC | EVQLVESGGDLVQPGGSLRLSCIASGFTFNNYWMS | 29 |
| | | WVRQAPGKGLEWVANIYRDGSEKYYLDSVEGRFTI | |
| | | SRDNAKNSLYLQMNSLRLEDTAVYYCARDPSTGDL | |
| | | DYWGQGTPVTVSSASTKGPSVFPLAPSSKSTSGGTA | |
| | | ALGCLVKDYFPEPVTVSWNSGALTSGVHTFPAVLQ | |
| | | SSGLYSLSSVVTVPSSSLGTQTYICNVNHKPSNTKV | |
| | | DKKVEPKSCDKTHTCPPCPAPELLGGPSVFLFPPKP | |
| | | KDTLMISRTPEVTCVVVDVSHEDPEVKFNWYVDGV | |
| | | EVHNAKTKPREEQYNSTYRVVSVLTVLHQDWLNG | |
| | | KEYKCKVSNKALPAPIEKTISKAKGQPREPQVYTLP | |
| | | PSREEMTKNQVSLTCLVKGFYPSDIAVEWESNGQPE | |
| | | NNYKTTPPVLDSDGSFFLYSKLTVDKSRWQQGNVF | |
| | | SCSVMHEALHNHYTQKSLSLSPGK | |
| | LC | DIQMTQSPLSLSVTLGQPASISCRSSQSLVYHDGNTY | 30 |
| | | LIWFQQRPGQSPRRLIYKVSNRDSGVPDRFSGSGSG | |
| | | TDFTLKISRVEAEDVGVYYCMQGSHWPRTFGPGTK | |
| | | VDIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B376 | HCDR1 | GFTFNNYWMS | 31 |
| m1 | HCDR2 | NIYRDGSEKY | 32 |
| | HCDR3 | EPSTGDLDY | 33 |
| | LCDR1 | RSSQSLVYHDGNTYLI | 34 |
| | LCDR2 | KVSNRDS | 35 |
| | LCDR3 | MQGSHWPRT | 36 |
| | VH | EVQLVESGGDLVQPGGSLRLSCIASGFTFNNYWMS | 37 |
| | | WVRQAPGKGLEWVANIYRDGSEKYYLDSVEGRFTI | |
| | | SRDNAKNSLYLQMNSLRLEDTAVYYCAREPSTGDL | |
| | | DYWGQGTPVTVSS | |
| | VL | DIQMTQSPLSLSVTLGQPASISCRSSQSLVYHDGNTY | 38 |
| | | LIWFQQRPGQSPRRLIYKVSNRDSGVPDRFSGSGSG | |
| | | TDFTLKISRVEAEDVGVYYCMQGSHWPRTFGPGTK | |
| | | VDIK | |
| | HC | EVQLVESGGDLVQPGGSLRLSCIASGFTFNNYWMS | 39 |
| | | WVRQAPGKGLEWVANIYRDGSEKYYLDSVEGRFTI | |
| | | SRDNAKNSLYLQMNSLRLEDTAVYYCAREPSTGDL | |
| | | DYWGQGTPVTVSSASTKGPSVFPLAPSSKSTSGGTA | |
| | | ALGCLVKDYFPEPVTVSWNSGALTSGVHTFPAVLQ | |
| | | SSGLYSLSSVVTVPSSSLGTQTYICNVNHKPSNTKV | |
| | | DKKVEPKSCDKTHTCPPCPAPELLGGPSVFLFPPKP | |
| | | KDTLMISRTPEVTCVVVDVSHEDPEVKFNWYVDGV | |
| | | EVHNAKTKPREEQYNSTYRVVSVLTVLHQDWLNG | |
| | | KEYKCKVSNKALPAPIEKTISKAKGQPREPQVYTLP | |
| | | PSREEMTKNQVSLTCLVKGFYPSDIAVEWESNGQPE | |
| | | NNYKTTPPVLDSDGSFFLYSKLTVDKSRWQQGNVF | |
| | | SCSVMHEALHNHYTQKSLSLSPGK | |
| | LC | DIQMTQSPLSLSVTLGQPASISCRSSQSLVYHDGNTY | 40 |
| | | LIWFQQRPGQSPRRLIYKVSNRDSGVPDRFSGSGSG | |
| | | TDFTLKISRVEAEDVGVYYCMQGSHWPRTFGPGTK | |
| | | VDIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B378 | HCDR1 | GFTFGTYAMN | 41 |
| | HCDR2 | YITTSSSHIY | 42 |
| | HCDR3 | DYGSSSSEDYYGMDV | 43 |
| | LCDR1 | RSGQSLLDSDDGNIYLD | 44 |
| | LCDR2 | TVSYRAS | 45 |
| | LCDR3 | MQRIDFPFT | 46 |
| | VH | EVQLVQSGGGLVKPGGSLRLSCAASGFTFGTYAMN | 47 |
| | | WVRQAPGKGLEWVSYITTSSSHIYYSDSVKGRFTIS | |
| | | RDNAKNSLYLQMNSLRAEDTAVYYCTRDYGSSSSE | |
| | | DYYGMDVWGQGTMVTVSS | |
| | VL | EIVMTQSPLYLPVTPGEPASISCRSGQSLLDSDDGNI | 48 |
| | | YLDWFLQKPGQSPQLLIYTVSYRASGVPDRFSGSGS | |
| | | GTDFTLKISRVEAEDVGVYYCMQRIDFPFTFGPGTK | |
| | | VDIK | |
| | HC | EVQLVQSGGGLVKPGGSLRLSCAASGFTFGTYAMN | 49 |
| | | WVRQAPGKGLEWVSYITTSSSHIYYSDSVKGRFTIS | |
| | | RDNAKNSLYLQMNSLRAEDTAVYYCTRDYGSSSSE | |
| | | DYYGMDVWGQGTMVTVSSASTKGPSVFPLAPSSKS | |
| | | TSGGTAALGCLVKDYFPEPVTVSWNSGALTSGVHT | |
| | | FPAVLQSSGLYSLSSVVTVPSSSLGTQTYICNVNHKP | |
| | | SNTKVDKKVEPKSCDKTHTCPPCPAPELLGGPSVFL | |
| | | FPPKPKDTLMISRTPEVTCVVVDVSHEDPEVKFNW | |
| | | YVDGVEVHNAKTKPREEQYNSTYRVVSVLTVLHQ | |
| | | DWLNGKEYKCKVSNKALPAPIEKTISKAKGQPREP | |
| | | QVYTLPPSREEMTKNQVSLTCLVKGFYPSDIAVEW | |
| | | ESNGQPENNYKTTPPVLDSDGSFFLYSKLTVDKSR | |
| | | WQQGNVFSCSVMHEALHNHYTQKSLSLSPGK | |
| | LC | EIVMTQSPLYLPVTPGEPASISCRSGQSLLDSDDGNI | 50 |
| | | YLDWFLQKPGQSPQLLIYTVSYRASGVPDRFSGSGS | |
| | | GTDFTLKISRVEAEDVGVYYCMQRIDFPFTFGPGTK | |
| | | VDIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B164 | HCDR1 | GFIFSTYWMS | 51 |
| | HCDR2 | NINPDGNEKY | 52 |
| | HCDR3 | DPISARFDF | 53 |
| | LCDR1 | RSSQSLEYSDGNTYLN | 54 |
| | LCDR2 | KVSNRDS | 55 |
| | LCDR3 | MQGTHWPPT | 56 |
| | VH | EVQLVESGGGLVQPGGSLRLSCAASGFIFSTYWMS | 57 |
| | | WVRQAPGKGLEWVANINPDGNEKYYVDSVEGRFT | |
| | | ISRDNAQDSLYLQMNSLRAEDTAVYYCARDPISARF | |
| | | DFWGQGTLVTVSS | |
| | VL | EIVMTQSPLSLPVTPGKPASISCRSSQSLEYSDGNTY | 58 |
| | | LNWFQQRPGQSPRRLIYKVSNRDSGVPDRFSGSGSG | |
| | | TDFTLKISRVEAEDVGVYYCMQGTHWPPTFGGGTK | |
| | | VDIK | |
| | HC | EVQLVESGGGLVQPGGSLRLSCAASGFIFSTYWMS | 59 |
| | | WVRQAPGKGLEWVANINPDGNEKYYVDSVEGRFT | |
| | | ISRDNAQDSLYLQMNSLRAEDTAVYYCARDPISARF | |
| | | DFWGQGTLVTVSSASTKGPSVFPLAPSSKSTSGGTA | |
| | | ALGCLVKDYFPEPVTVSWNSGALTSGVHTFPAVLQ | |
| | | SSGLYSLSSVVTVPSSSLGTQTYICNVNHKPSNTKV | |
| | | DKKVEPKSCDKTHTCPPCPAPELLGGPSVFLFPPKP | |
| | | KDTLMISRTPEVTCVVVDVSHEDPEVKFNWYVDGV | |
| | | EVHNAKTKPREEQYNSTYRVVSVLTVLHQDWLNG | |
| | | KEYKCKVSNKALPAPIEKTISKAKGQPREPQVYTLP | |
| | | PSREEMTKNQVSLTCLVKGFYPSDIAVEWESNGQPE | |
| | | NNYKTTPPVLDSDGSFFLYSKLTVDKSRWQQGNVF | |
| | | SCSVMHEALHNHYTQKSLSLSPGK | |
| | LC | EIVMTQSPLSLPVTPGKPASISCRSSQSLEYSDGNTY | 60 |
| | | LNWFQQRPGQSPRRLIYKVSNRDSGVPDRFSGSGSG | |
| | | TDFTLKISRVEAEDVGVYYCMQGTHWPPTFGGGTK | |
| | | VDIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B164 | HCDR1 | GFIFSTYWMS | 61 |
| m1 | HCDR2 | NINPDGNEKY | 62 |
| | HCDR3 | EPISARFDF | 63 |
| | LCDR1 | RSSQSLEYSDGNTYLN | 64 |
| | LCDR2 | KVSNRDS | 65 |
| | LCDR3 | MQGTHWPPT | 66 |
| | VH | EVQLVESGGGLVQPGGSLRLSCAASGFIFSTYWMS | 67 |
| | | WVRQAPGKGLEWVANINPDGNEKYYVDSVEGRFT | |
| | | ISRDNAQDSLYLQMNSLRAEDTAVYYCAREPISARF | |
| | | DFWGQGTLVTVSS | |
| | VL | EIVMTQSPLSLPVTPGKPASISCRSSQSLEYSDGNTY | 68 |
| | | LNWFQQRPGQSPRRLIYKVSNRDSGVPDRFSGSGSG | |
| | | TDFTLKISRVEAEDVGVYYCMQGTHWPPTFGGGTK | |
| | | VDI | |
| | HC | EVQLVESGGGLVQPGGSLRLSCAASGFIFSTYWMS | 69 |
| | | WVRQAPGKGLEWVANINPDGNEKYYVDSVEGRFT | |
| | | ISRDNAQDSLYLQMNSLRAEDTAVYYCAREPISARF | |
| | | DFWGQGTLVTVSSASTKGPSVFPLAPSSKSTSGGTA | |
| | | ALGCLVKDYFPEPVTVSWNSGALTSGVHTFPAVLQ | |
| | | SSGLYSLSSVVTVPSSSLGTQTYICNVNHKPSNTKV | |
| | | DKKVEPKSCDKTHTCPPCPAPELLGGPSVFLFPPKP | |
| | | KDTLMISRTPEVTCVVVDVSHEDPEVKFNWYVDGV | |
| | | EVHNAKTKPREEQYNSTYRVVSVLTVLHQDWLNG | |
| | | KEYKCKVSNKALPAPIEKTISKAKGQPREPQVYTLP | |
| | | PSREEMTKNQVSLTCLVKGFYPSDIAVEWESNGQPE | |
| | | NNYKTTPPVLDSDGSFFLYSKLTVDKSRWQQGNVF | |
| | | SCSVMHEALHNHYTQKSLSLSPGK | |
| | LC | EIVMTQSPLSLPVTPGKPASISCRSSQSLEYSDGNTY | 70 |
| | | LNWFQQRPGQSPRRLIYKVSNRDSGVPDRFSGSGSG | |
| | | TDFTLKISRVEAEDVGVYYCMQGTHWPPTFGGGTK | |
| | | VDIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B312 | HCDR1 | GFTFSRHTMN | 71 |
| | HCDR2 | SISGSGGSTY | 72 |
| | HCDR3 | ESYDSSGVWYFDL | 73 |
| | LCDR1 | SGHKLGDKYAC | 74 |
| | LCDR2 | QDSKRPS | 75 |
| | LCDR3 | QAWDSITAWV | 76 |
| | VH | EVQLVESGGGLVQSGGSLRISCVASGFTFSRHTMN | 77 |
| | | WVRQAPGKGLEWVSSISGSGGSTYYADSVKGRLTV | |
| | | SRDNSKNTLYLQMNSLRAEDTAVYYCAKESYDSSG | |
| | | VWYFDLWGRGTLVTVSS | |
| | VL | SSELTQPPSMSVSPGQTANIACSGHKLGDKYACWY | 78 |
| | | QQKPGQSPVLVIYQDSKRPSGIPERFSGSNSGNTATL | |
| | | TISGTQAMDEADYYCQAWDSITAWVFGGGTKVTV | |
| | | L | |
| | HC | EVQLVESGGGLVQSGGSLRISCVASGFTFSRHTMN | 79 |
| | | WVRQAPGKGLEWVSSISGSGGSTYYADSVKGRLTV | |
| | | SRDNSKNTLYLQMNSLRAEDTAVYYCAKESYDSSG | |
| | | VWYFDLWGRGTLVTVSSASTKGPSVFPLAPSSKSTS | |
| | | GGTAALGCLVKDYFPEPVTVSWNSGALTSGVHTFP | |
| | | AVLQSSGLYSLSSVVTVPSSSLGTQTYICNVNHKPS | |
| | | NTKVDKKVEPKSCDKTHTCPPCPAPELLGGPSVFLF | |
| | | PPKPKDTLMISRTPEVTCVVVDVSHEDPEVKFNWY | |
| | | VDGVEVHNAKTKPREEQYNSTYRVVSVLTVLHQD | |
| | | WLNGKEYKCKVSNKALPAPIEKTISKAKGQPREPQ | |
| | | VYTLPPSREEMTKNQVSLTCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGK | |
| | LC | SSELTQPPSMSVSPGQTANIACSGHKLGDKYACWY | 80 |
| | | QQKPGQSPVLVIYQDSKRPSGIPERFSGSNSGNTATL | |
| | | TISGTQAMDEADYYCQAWDSITAWVFGGGTKVTV | |
| | | LGQPKAAPSVTLFPPSSEELQANKATLVCLISDFYPG | |
| | | AVTVAWKADSSPVKAGVETTTPSKQSNNKYAASS | |
| | | YLSLTPEQWKSHRSYSCQVTHEGSTVEKTVAPTECS | |
| CD9B312 | HCDR1 | GFTFSRHTMN | 81 |
| m1 | HCDR2 | SISGSGGSTY | 82 |
| | HCDR3 | ESYDSSGVWYFDL | 83 |
| | LCDR1 | SGHKLGDKYAS | 84 |
| | LCDR2 | QDSKRPS | 85 |
| | LCDR3 | QAWDSITAWV | 86 |
| | VH | EVQLVESGGGLVQSGGSLRISCVASGFTFSRHTMN | 87 |
| | | WVRQAPGKGLEWVSSISGSGGSTYYADSVKGRLTV | |
| | | SRDNSKNTLYLQMNSLRAEDTAVYYCAKESYDSSG | |
| | | VWYFDLWGRGTLVTVSS | |
| | VL | SSELTQPPSMSVSPGQTANIACSGHKLGDKYASWY | 88 |
| | | QQKPGQSPVLVIYQDSKRPSGIPERFSGSNSGNTATL | |
| | | TISGTQAMDEADYYCQAWDSITAWVFGGGTKVTV | |
| | | L | |
| | HC | EVQLVESGGGLVQSGGSLRISCVASGFTFSRHTMN | 89 |
| | | WVRQAPGKGLEWVSSISGSGGSTYYADSVKGRLTV | |
| | | SRDNSKNTLYLQMNSLRAEDTAVYYCAKESYDSSG | |
| | | VWYFDLWGRGTLVTVSSASTKGPSVFPLAPSSKSTS | |
| | | GGTAALGCLVKDYFPEPVTVSWNSGALTSGVHTFP | |
| | | AVLQSSGLYSLSSVVTVPSSSLGTQTYICNVNHKPS | |
| | | NTKVDKKVEPKSCDKTHTCPPCPAPELLGGPSVFLF | |
| | | PPKPKDTLMISRTPEVTCVVVDVSHEDPEVKFNWY | |
| | | VDGVEVHNAKTKPREEQYNSTYRVVSVLTVLHQD | |
| | | WLNGKEYKCKVSNKALPAPIEKTISKAKGQPREPQ | |
| | | VYTLPPSREEMTKNQVSLTCLVKGFYPSDIAVEWES | |
| | | NGQPENNYKTTPPVLDSDGSFFLYSKLTVDKSRWQ | |
| | | QGNVFSCSVMHEALHNHYTQKSLSLSPGK | |
| | LC | SSELTQPPSMSVSPGQTANIACSGHKLGDKYASWY | 90 |
| | | QQKPGQSPVLVIYQDSKRPSGIPERFSGSNSGNTATL | |
| | | TISGTQAMDEADYYCQAWDSITAWVFGGGTKVTV | |
| | | LGQPKAAPSVTLFPPSSEELQANKATLVCLISDFYPG | |
| | | AVTVAWKADSSPVKAGVETTTPSKQSNNKYAASS | |
| | | YLSLTPEQWKSHRSYSCQVTHEGSTVEKTVAPTECS | |
| CD9B292 | HCDR1 | GFTFNSYNMN | 91 |
| | HCDR2 | YISSSGYTIY | 92 |
| | HCDR3 | DRYNYDNGGYHYYTGMDV | 93 |
| | LCDR1 | SGDKLGDKFAC | 94 |
| | LCDR2 | QDSKRPS | 95 |
| | LCDR3 | QAWDSTTAWV | 96 |
| | VH | EVQLVESGGGLVQPGGSLRLSCAASGFTFNSYNMN | 97 |
| | | WVRQAPGKGLEWVSYISSSGYTIYYADSVEGRFTIS | |
| | | RDNAKNSLYLQMDSLRDEDTAIYYCARDRYNYDN | |
| | | GGYHYYTGMDVWGQGTMVTVSS | |
| | VL | SSELTQPPSVSVSPGQTASITCSGDKLGDKFACWYQ | 98 |
| | | QKPGQSPVLVIFQDSKRPSGIPERFSGSNFGNTATLTI | |
| | | SGTQAMDEADYFCQAWDSTTAWVFGGGTKVTVL | |
| | HC | EVQLVESGGGLVQPGGSLRLSCAASGFTFNSYNMN | 99 |
| | | WVRQAPGKGLEWVSYISSSGYTIYYADSVEGRFTIS | |
| | | RDNAKNSLYLQMDSLRDEDTAIYYCARDRYNYDN | |
| | | GGYHYYTGMDVWGQGTMVTVSSASTKGPSVFPLA | |
| | | PSSKSTSGGTAALGCLVKDYFPEPVTVSWNSGALTS | |
| | | GVHTFPAVLQSSGLYSLSSVVTVPSSSLGTQTYICNV | |
| | | NHKPSNTKVDKKVEPKSCDKTHTCPPCPAPELLGGP | |
| | | SVFLFPPKPKDTLMISRTPEVTCVVVDVSHEDPEVK | |
| | | FNWYVDGVEVHNAKTKPREEQYNSTYRVVSVLTV | |
| | | LHQDWLNGKEYKCKVSNKALPAPIEKTISKAKGQP | |
| | | REPQVYTLPPSREEMTKNQVSLTCLVKGFYPSDIAV | |
| | | EWESNGQPENNYKTTPPVLDSDGSFFLYSKLTVDKS | |
| | | RWQQGNVFSCSVMHEALHNHYTQKSLSLSPGK | |
| | LC | SSELTQPPSVSVSPGQTASITCSGDKLGDKFACWYQ | 100 |
| | | QKPGQSPVLVIFQDSKRPSGIPERFSGSNFGNTATLTI | |
| | | SGTQAMDEADYFCQAWDSTTAWVFGGGTKVTVL | |
| | | GQPKAAPSVTLFPPSSEELQANKATLVCLISDFYPGA | |
| | | VTVAWKADSSPVKAGVETTTPSKQSNNKYAASSYL | |
| | | SLTPEQWKSHRSYSCQVTHEGSTVEKTVAPTECS | |
| CD9B292 | HCDR1 | GFTFNSYNMN | 101 |
| m1 | HCDR2 | YISSSGYTIY | 102 |
| | HCDR3 | DRYNYDNGGYHYYTGMDV | 103 |
| | LCDR1 | SGDKLGDKFAS | 104 |
| | LCDR2 | QDSKRPS | 105 |
| | LCDR3 | QAWDSTTAWV | 106 |
| | VH | EVQLVESGGGLVQPGGSLRLSCAASGFTFNSYNMN | 107 |
| | | WVRQAPGKGLEWVSYISSSGYTIYYADSVEGRFTIS | |
| | | RDNAKNSLYLQMDSLRDEDTAIYYCARDRYNYDN | |
| | | GGYHYYTGMDVWGQGTMVTVSS | |
| | VL | SSELTQPPSVSVSPGQTASITCSGDKLGDKFASWYQ | 108 |
| | | QKPGQSPVLVIFQDSKRPSGIPERFSGSNFGNTATLTI | |
| | | SGTQAMDEADYFCQAWDSTTAWVFGGGTKVTVL | |
| | HC | EVQLVESGGGLVQPGGSLRLSCAASGFTFNSYNMN | 109 |
| | | WVRQAPGKGLEWVSYISSSGYTIYYADSVEGRFTIS | |
| | | RDNAKNSLYLQMDSLRDEDTAIYYCARDRYNYDN | |
| | | GGYHYYTGMDVWGQGTMVTVSSASTKGPSVFPLA | |
| | | PSSKSTSGGTAALGCLVKDYFPEPVTVSWNSGALTS | |
| | | GVHTFPAVLQSSGLYSLSSVVTVPSSSLGTQTYICNV | |
| | | NHKPSNTKVDKKVEPKSCDKTHTCPPCPAPELLGGP | |
| | | SVFLFPPKPKDTLMISRTPEVTCVVVDVSHEDPEVK | |
| | | FNWYVDGVEVHNAKTKPREEQYNSTYRVVSVLTV | |
| | | LHQDWLNGKEYKCKVSNKALPAPIEKTISKAKGQP | |
| | | REPQVYTLPPSREEMTKNQVSLTCLVKGFYPSDIAV | |
| | | EWESNGQPENNYKTTPPVLDSDGSFFLYSKLTVDKS | |
| | | RWQQGNVFSCSVMHEALHNHYTQKSLSLSPGK | |
| | LC | SSELTQPPSVSVSPGQTASITCSGDKLGDKFASWYQ | 110 |
| | | QKPGQSPVLVIFQDSKRPSGIPERFSGSNFGNTATLTI | |
| | | SGTQAMDEADYFCQAWDSTTAWVFGGGTKVTVL | |
| | | GQPKAAPSVTLFPPSSEELQANKATLVCLISDFYPGA | |
| | | VTVAWKADSSPVKAGVETTTPSKQSNNKYAASSYL | |
| | | SLTPEQWKSHRSYSCQVTHEGSTVEKTVAPTECS | |
| CD9B1256 | HCDR1 | SYYWS | 111 |
| | HCDR2 | RIYSSGSTNYNPSLKS | 112 |
| | HCDR3 | GEYSGDLGGYGLDV | 113 |
| | LCDR1 | RSSQSLLDSDDGNTYLD | 114 |
| | LCDR2 | TLSYRAS | 115 |
| | LCDR3 | MQRMEFPLT | 116 |
| | VH | EVQLVESGPGLVEPSETLSLTCTVSGGSISSYYWSWI | 117 |
| | | RQPAGKGLEWIGRIYSSGSTNYNPSLKSRVTMSVDT | |
| | | SENQFSLNLNSVTAADTAKYYCARGEYSGDLGGYG | |
| | | LDVWGQGTMVTVSS | |
| | VL | EIVMTQSPLSLSVTPGEPASISCRSSQSLLDSDDGNT | 118 |
| | | YLDWYLQKPGQSPQLLIQTLSYRASGVPDRFSGRGS | |
| | | GTDFTLKISRVEAEDVGIYYCMQRMEFPLTFGGGTK | |
| | | VDIK | |
| | HC | EVQLVESGPGLVEPSETLSLTCTVSGGSISSYYWSWI | 119 |
| | | RQPAGKGLEWIGRIYSSGSTNYNPSLKSRVTMSVDT | |
| | | SENQFSLNLNSVTAADTAKYYCARGEYSGDLGGYG | |
| | | LDVWGQGTMVTVSSASTKGPSVFPLAPSSKSTSGG | |
| | | TAALGCLVKDYFPEPVTVSWNSGALTSGVHTFPAV | |
| | | LQSSGLYSLSSVVTVPSSSLGTQTYICNVNHKPSNTK | |
| | | VDKKVEPKSCDKTHTCPPCPAPEAAGGPSVFLFPPK | |
| | | PKDTLMISRTPEVTCVVVSVSHEDPEVKFNWYVDG | |
| | | VEVHNAKTKPREEQYNSTYRVVSVLTVLHQDWLN | |
| | | GKEYKCKVSNKALPAPIEKTISKAKGQPREPQVYTL | |
| | | PPSREEMTKNQVSLTCLVKGFYPSDIAVEWESNGQP | |
| | | ENNYKTTPPVLDSDGSFFLYSKLTVDKSRWQQGNV | |
| | | FSCSVMHEALHNHYTQKSLSLSPGK | |
| | LC | EIVMTQSPLSLSVTPGEPASISCRSSQSLLDSDDGNT | 120 |
| | | YLDWYLQKPGQSPQLLIQTLSYRASGVPDRFSGRGS | |
| | | GTDFTLKISRVEAEDVGIYYCMQRMEFPLTFGGGTK | |
| | | VDIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B1218 | HCDR1 | SYYWS | 121 |
| | HCDR2 | RIHTSGNTNYNPSLKS | 122 |
| | HCDR3 | GEYSGDLGGYGLDV | 123 |
| | LCDR1 | RSSQSLLDRDDGNTYLD | 124 |
| | LCDR2 | TLSYRAS | 125 |
| | LCDR3 | MQRIEFPLT | 126 |
| | VH | QVQLQESGPGLVKPSETLSLTCTVSGVSISSYYWSW | 127 |
| | | IRQPAGKGLEWIGRIHTSGNTNYNPSLKSRATMSGD | |
| | | TSKNHFSLKLTSVTAADTAVYYCARGEYSGDLGGY | |
| | | GLDVWGQGTMVTVSS | |
| | VL | DIVMTQTPLSLPVTPGEPASISCRSSQSLLDRDDGNT | 128 |
| | | YLDWYLQKPGQSPQLLIYTLSYRASGVPDRFSGSGS | |
| | | GTDFTLKISRVEAEDVGVYYCMQRIEFPLTFGGGTK | |
| | | VEIK | |
| | HC | QVQLQESGPGLVKPSETLSLTCTVSGVSISSYYWSW | 129 |
| | | IRQPAGKGLEWIGRIHTSGNTNYNPSLKSRATMSGD | |
| | | TSKNHFSLKLTSVTAADTAVYYCARGEYSGDLGGY | |
| | | GLDVWGQGTMVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPSNT | |
| | | KVDKKVEPKSCDKTHTCPPCPAPEAAGGPSVFLFPP | |
| | | KPKDTLMISRTPEVTCVVVSVSHEDPEVKFNWYVD | |
| | | GVEVHNAKTKPREEQYNSTYRVVSVLTVLHQDWL | |
| | | NGKEYKCKVSNKALPAPIEKTISKAKGQPREPQVYT | |
| | | LPPSREEMTKNQVSLTCLVKGFYPSDIAVEWESNGQ | |
| | | PENNYKTTPPVLDSDGSFFLYSKLTVDKSRWQQGN | |
| | | VFSCSVMHEALHNHYTQKSLSLSPGK | |
| | LC | DIVMTQTPLSLPVTPGEPASISCRSSQSLLDRDDGNT | 130 |
| | | YLDWYLQKPGQSPQLLIYTLSYRASGVPDRFSGSGS | |
| | | GTDFTLKISRVEAEDVGVYYCMQRIEFPLTFGGGTK | |
| | | VEIKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFNR | |
| | | GEC | |
| CD9B1315 | HCDR1 | SYYWS | 131 |
| | HCDR2 | RIYTRGSTHYNPSLKS | 132 |
| | HCDR3 | GEYSGDLGGYGLDV | 133 |
| | LCDR1 | RSSQSLLDRDDGKTYLD | 134 |
| | LCDR2 | TLSDRAS | 135 |
| | LCDR3 | MQRIEFPLT | 136 |
| | VH | EVQLVESGPELVKPSETLSLTCTVSGVSISSYYWSWI | 137 |
| | | RQPAGKGLEWIGRIYTRGSTHYNPSLKSRVTMSEDT | |
| | | SKNQFFLKLGSVTAADTAVYYCARGEYSGDLGGY | |
| | | GLDVWGQGTMVTVSS | |
| | VL | DIVMTQSPLSLPVTPGEPASISCRSSQSLLDRDDGKT | 138 |
| | | YLDWYLQKPGQSPQLLIYTLSDRASGVPDRFSGSGS | |
| | | GTDFTLKISRVEADDVGVYYCMQRIEFPLTFGGGTK | |
| | | LEIK | |
| | HC | EVQLVESGPELVKPSETLSLTCTVSGVSISSYYWSWI | 139 |
| | | RQPAGKGLEWIGRIYTRGSTHYNPSLKSRVTMSEDT | |
| | | SKNQFFLKLGSVTAADTAVYYCARGEYSGDLGGY | |
| | | GLDVWGQGTMVTVSSASTKGPSVFPLAPSSKSTSG | |
| | | GTAALGCLVKDYFPEPVTVSWNSGALTSGVHTFPA | |
| | | VLQSSGLYSLSSVVTVPSSSLGTQTYICNVNHKPSNT | |
| | | KVDKKVEPKSCDKTHTCPPCPAPEAAGGPSVFLFPP | |
| | | KPKDTLMISRTPEVTCVVVSVSHEDPEVKFNWYVD | |
| | | GVEVHNAKTKPREEQYNSTYRVVSVLTVLHQDWL | |
| | | NGKEYKCKVSNKALPAPIEKTISKAKGQPREPQVYT | |
| | | LPPSREEMTKNQVSLTCLVKGFYPSDIAVEWESNGQ | |
| | | PENNYKTTPPVLDSDGSFFLYSKLTVDKSRWQQGN | |
| | | VFSCSVMHEALHNHYTQKSLSLSPGK | |
| | LC | DIVMTQSPLSLPVTPGEPASISCRSSQSLLDRDDGKT | 140 |
| | | YLDWYLQKPGQSPQLLIYTLSDRASGVPDRFSGSGS | |
| | | GTDFTLKISRVEADDVGVYYCMQRIEFPLTFGGGTK | |
| | | LEIKRTVAAPSVFIFPPSDEQLKSGTASVVCLLNNFY | |

PREAKVQWKVDNALQSGNSQESVTEQDSKDSTYSL
SSTLTLSKADYEKHKVYACEVTHQGLSSPVTKSFNR
GEC

TABLE 4
Linker Sequences

| Linker ID | Amino Acid Sequence | SEQ ID NO: |
|---|---|---|
| Linker 1 | GGSEGKSSGSGSESKSTGGS | 141 |
| Linker 2 | GGGSGGGS | 142 |
| Linker 3 | GGGSGGGSGGGS | 143 |
| Linker 4 | GGGSGGGSGGGSGGGS | 144 |
| Linker 5 | GGGSGGGSGGGSGGGSGGGS | 145 |
| Linker 6 | GGGGSGGGGSGGGGS | 146 |
| Linker 7 | GGGGSGGGGSGGGGSGGGGS | 147 |
| Linker 8 | GGGGSGGGGSGGGGSGGGGSGGGGS | 148 |
| Linker 9 | GSTSGSGKPGSGEGSTKG | 149 |
| Linker 10 | IRPRAIGGSKPRVA | 150 |
| Linker 11 | GKGGSGKGGSGKGGS | 151 |
| Linker 12 | GGKGSGGKGSGGKGS | 152 |
| Linker 13 | GGGKSGGGKSGGGKS | 153 |
| Linker 14 | GKGKSGKGKSGKGKS | 154 |
| Linker 15 | GGGKSGGKGSGKGGS | 155 |
| Linker 16 | GKPGSGKPGSGKPGS | 156 |
| Linker 17 | GKPGSGKPGSGKPGSGKPGS | 157 |
| Linker 18 | GKGKSGKGKSGKGKSGKGKS | 158 |
| Linker 19 | STAGDTHLGGEDFD | 159 |
| Linker 20 | GEGGSGEGGSGEGGS | 160 |
| Linker 21 | GGEGSGGEGSGGEGS | 161 |
| Linker 22 | GEGESGEGESGEGES | 162 |
| Linker 23 | GGGESGGEGSGEGGS | 163 |
| Linker 24 | GEGESGEGESGEGESGEGES | 164 |
| Linker 25 | GSTSGSGKPGSGEGSTKG | 165 |
| Linker 26 | PRGASKSGSASQTGSAPGS | 166 |
| Linker 27 | GTAAAGAGAAGGAAAGAAG | 167 |
| Linker 28 | GTSGSSGSGSGGSGSGGGG | 168 |
| Linker 29 | GKPGSGKPGSGKPGSGKPGS | 169 |
| Linker 30 | GSGS | 170 |
| Linker 31 | APAPAPAPAP | 171 |
| Linker 32 | APAPAPAPAPAPAPAPAPAP | 172 |
| Linker 33 | AEAAAKEAAAKEAAAAKEAAAAKEAAAAKAAA | 173 |

### Example 2

The invention includes at least the following numbered clauses:

Clause 1: An isolated protein comprising an antigen binding domain that binds Cluster of Differentiation 79B protein (CD79b), wherein the antigen binding domain that binds CD79b comprises at least one complementarity determining region (CDR) selected from the group consisting of a heavy chain complementarity determining region (HCDR) 1, a HCDR2, a HCDR3, a light chain complementarity determining region (LCDR) 1, a LCDR2 and a LCDR 3, wherein
(a) the HCDR1 is selected from the group consisting of SEQ ID NOs: 1, 11, 21, 31, 41, 51, 61, 71, 81, 91, 101, 111, 121, and 131;
(b) the HCDR2 is selected from the group consisting of SEQ ID NOs: 2, 12, 22, 32, 42, 52, 62, 72, 82, 92, 102, 112, 122, and 132;
(c) the HCDR3 is selected from the group consisting of SEQ ID NOs: 3, 13, 23, 33, 43, 53, 63, 73, 83, 93, 103, 113, 123, and 133;
(d) the LCDR1 is selected from the group consisting of SEQ ID NOs: 4, 14, 24, 34, 44, 54, 64, 74, 84, 94, 104, 114, 124, and 134;
(e) the LCDR2 is selected from the group consisting of SEQ ID NOs: 5, 15, 25, 35, 45, 55, 65, 75, 85, 95, 105, 115, 125, and 135; and
(f) the LCDR3 is selected from the group consisting of SEQ ID NOs: 6, 16, 26, 36, 46, 56, 66, 76, 86, 96, 106, 116, 126, and 136.

Clause 2: The isolated protein of clause 1, wherein the antigen binding domain that binds CD79b comprises a HCDR1, a HCDR2 and a HCDR3.

Clause 3: The isolated protein of clause 2, wherein the antigen binding domain that binds CD79b comprises:
(g) a HCDR1 of SEQ ID NO: 1, a HCDR2 of SEQ ID NO: 2, and a HCDR3 of SEQ ID NO: 3;
(h) a HCDR1 of SEQ ID NO: 11, a HCDR2 of SEQ ID NO: 12, and a HCDR3 of SEQ ID NO: 13;
(i) a HCDR1 of SEQ ID NO: 21, a HCDR2 of SEQ ID NO: 22, and a HCDR3 of SEQ ID NO: 23;
(j) a HCDR1 of SEQ ID NO: 31, a HCDR2 of SEQ ID NO: 32, and a HCDR3 of SEQ ID NO: 33;
(k) a HCDR1 of SEQ ID NO: 41, a HCDR2 of SEQ ID NO: 42, and a HCDR3 of SEQ ID NO: 43;
(l) a HCDR1 of SEQ ID NO: 51, a HCDR2 of SEQ ID NO: 52, and a HCDR3 of SEQ ID NO: 53;
(m) a HCDR1 of SEQ ID NO: 61, a HCDR2 of SEQ ID NO: 62, and a HCDR3 of SEQ ID NO: 63;
(n) a HCDR1 of SEQ ID NO: 71, a HCDR2 of SEQ ID NO: 72, and a HCDR3 of SEQ ID NO: 73;
(o) a HCDR1 of SEQ ID NO: 81, a HCDR2 of SEQ ID NO: 82, and a HCDR3 of SEQ ID NO: 83;
(p) a HCDR1 of SEQ ID NO: 91, a HCDR2 of SEQ ID NO: 92, and a HCDR3 of SEQ ID NO: 93;
(q) a HCDR1 of SEQ ID NO: 101, a HCDR2 of SEQ ID NO: 102, and a HCDR3 of SEQ ID NO: 103;
(r) a HCDR1 of SEQ ID NO: 111, a HCDR2 of SEQ ID NO: 112, and a HCDR3 of SEQ ID NO: 113;
(s) a HCDR1 of SEQ ID NO: 121, a HCDR2 of SEQ ID NO: 122, and a HCDR3 of SEQ ID NO: 123; or
(t) a HCDR1 of SEQ ID NO: 131, a HCDR2 of SEQ ID NO: 132, and a HCDR3 of SEQ ID NO: 133.

Clause 4: The isolated protein of any of clauses 1-3, wherein the antigen binding domain that binds CD79b comprises a LCDR1, a LCDR2 and a LCDR3.

Clause 5: The isolated protein of clause 4, wherein the antigen binding domain that binds CD79b comprises:
(u) a LCDR1 of SEQ ID NO: 4, a LCDR2 of SEQ ID NO:5, and a LCDR3 of SEQ ID NO:6;
(v) a LCDR1 of SEQ ID NO: 14, a LCDR2 of SEQ ID NO: 15, and a LCDR3 of SEQ ID NO: 16;
(w) a LCDR1 of SEQ ID NO: 24, a LCDR2 of SEQ ID NO:25, and a LCDR3 of SEQ ID NO:26;
(x) a LCDR1 of SEQ ID NO: 34, a LCDR2 of SEQ ID NO:35, and a LCDR3 of SEQ ID NO:36;
(y) a LCDR1 of SEQ ID NO: 44, a LCDR2 of SEQ ID NO:45, and a LCDR3 of SEQ ID NO:46;
(z) a LCDR1 of SEQ ID NO: 54, a LCDR2 of SEQ ID NO:55, and a LCDR3 of SEQ ID NO:56;
(aa) a LCDR1 of SEQ ID NO: 64, a LCDR2 of SEQ ID NO:65, and a LCDR3 of SEQ ID NO: 66;
(bb) a LCDR1 of SEQ ID NO: 74, a LCDR2 of SEQ ID NO:75, and a LCDR3 of SEQ ID NO: 76;
(cc) a LCDR1 of SEQ ID NO: 84, a LCDR2 of SEQ ID NO:85, and a LCDR3 of SEQ ID NO: 86;
(dd) a LCDR1 of SEQ ID NO: 94, a LCDR2 of SEQ ID NO:95, and a LCDR3 of SEQ ID NO: 96;
(ee) a LCDR1 of SEQ ID NO: 104, a LCDR2 of SEQ ID NO: 105, and a LCDR3 of SEQ ID NO: 106;
(ff) a LCDR1 of SEQ ID NO: 114, a LCDR2 of SEQ ID NO: 115, and a LCDR3 of SEQ ID NO:116;
(gg) a LCDR1 of SEQ ID NO: 124, a LCDR2 of SEQ ID NO: 125, and a LCDR3 of SEQ ID NO:126; or
(hh) a LCDR1 of SEQ ID NO: 134, a LCDR2 of SEQ ID NO: 135, and a LCDR3 of SEQ ID NO: 136.

Clause 6: The isolated protein of clause 1, wherein the antigen binding domain that binds CD79b comprises a HCDR1, a HCDR2, a HCDR3, LCDR1, a LCDR2, and a LCDR3.

Clause 7: The isolated protein of clause 6, wherein the antigen binding domain that binds CD79b comprises:
(ii) a HCDR1 of SEQ ID NO: 1, a HCDR2 of SEQ ID NO: 2, a HCDR3 of SEQ ID NO: 3; a LCDR1 of SEQ ID NO: 4, a LCDR2 of SEQ ID NO:5, and a LCDR3 of SEQ ID NO:6;
(jj) a HCDR1 of SEQ ID NO: 11, a HCDR2 of SEQ ID NO: 12, a HCDR3 of SEQ ID NO: 13; a LCDR1 of SEQ ID NO: 14, a LCDR2 of SEQ ID NO: 15, and a LCDR3 of SEQ ID NO: 16;
(kk) a HCDR1 of SEQ ID NO: 21, a HCDR2 of SEQ ID NO: 22, a HCDR3 of SEQ ID NO: 23; a LCDR1 of SEQ ID NO: 24, a LCDR2 of SEQ ID NO:25, and a LCDR3 of SEQ ID NO:26;
(11) a HCDR1 of SEQ ID NO: 31, a HCDR2 of SEQ ID NO: 32, a HCDR3 of SEQ ID NO: 33; a LCDR1 of SEQ ID NO: 34, a LCDR2 of SEQ ID NO:35, and a LCDR3 of SEQ ID NO:36;
(mm) a HCDR1 of SEQ ID NO: 41, a HCDR2 of SEQ ID NO: 42, a HCDR3 of SEQ ID NO: 43; a LCDR1 of SEQ ID NO: 44, a LCDR2 of SEQ ID NO:45, and a LCDR3 of SEQ ID NO:46;
(nn) a HCDR1 of SEQ ID NO: 51, a HCDR2 of SEQ ID NO: 52, a HCDR3 of SEQ ID NO: 53; a LCDR1 of SEQ ID NO: 54, a LCDR2 of SEQ ID NO:55, and a LCDR3 of SEQ ID NO:56;
(oo) a HCDR1 of SEQ ID NO: 61, a HCDR2 of SEQ ID NO: 62, a HCDR3 of SEQ ID NO: 63; a LCDR1 of SEQ ID NO: 64, a LCDR2 of SEQ ID NO:65, and a LCDR3 of SEQ ID NO:66;
(pp) a HCDR1 of SEQ ID NO: 71, a HCDR2 of SEQ ID NO: 72, a HCDR3 of SEQ ID NO: 73; a LCDR1 of SEQ ID NO: 74, a LCDR2 of SEQ ID NO:75, and a LCDR3 of SEQ ID NO:76;
(qq) a HCDR1 of SEQ ID NO: 81, a HCDR2 of SEQ ID NO: 82, a HCDR3 of SEQ ID NO: 83; a LCDR1 of SEQ ID NO: 84, a LCDR2 of SEQ ID NO:85, and a LCDR3 of SEQ ID NO:86;
(rr) a HCDR1 of SEQ ID NO: 91, a HCDR2 of SEQ ID NO: 92, a HCDR3 of SEQ ID NO: 93; a LCDR1 of SEQ ID NO: 94, a LCDR2 of SEQ ID NO:95, and a LCDR3 of SEQ ID NO:96;
(ss) a HCDR1 of SEQ ID NO: 101, a HCDR2 of SEQ ID NO: 102, a HCDR3 of SEQ ID NO: 103; a LCDR1 of SEQ ID NO: 104, a LCDR2 of SEQ ID NO:105, and a LCDR3 of SEQ ID NO: 106;
(tt) a HCDR1 of SEQ ID NO: 111, a HCDR2 of SEQ ID NO: 112, a HCDR3 of SEQ ID NO: 113; a LCDR1 of SEQ ID NO: 114, a LCDR2 of SEQ ID NO:115, and a LCDR3 of SEQ ID NO: 116;
(uu) a HCDR1 of SEQ ID NO: 121, a HCDR2 of SEQ ID NO: 122, a HCDR3 of SEQ ID NO: 123; a LCDR1 of SEQ ID NO: 124, a LCDR2 of SEQ ID NO:125, and a LCDR3 of SEQ ID NO: 126; or
(vv) a HCDR1 of SEQ ID NO: 131, a HCDR2 of SEQ ID NO: 132, a HCDR3 of SEQ ID NO: 133; a LCDR1 of SEQ ID NO: 134, a LCDR2 of SEQ ID NO:135, and a LCDR3 of SEQ ID NO:136.

Clause 8: The isolated protein of any of clauses 1-7, wherein the antigen binding domain that binds CD79b comprises a heavy chain variable region (VH), wherein the VH comprises the HCDR1, HDR2 and HCDR3.

Clause 9: The isolated protein of clause 8, wherein the VH is selected from the group consisting of SEQ ID NOs: 7, 17, 27, 37, 47, 57, 67, 77, 87, 97, 107, 117, 127, and 137.

Clause 10: The isolated protein of any of clauses 1-9, wherein the antigen binding domain that binds CD79b comprises a light chain variable region (VL), wherein the VL comprises the LCDR1, LDR2 and LCDR3.

Clause 11: The isolated protein of clause 10, wherein the VL is selected from the group consisting of SEQ ID NOs: 8, 18, 28, 38, 48, 58, 68, 78, 88, 98, 108, 118, 128, and 138.

Clause 12: The isolated protein of any of clauses 1-11, wherein the antigen binding domain that binds CD79b comprises a heavy chain variable region (VH) and light chain variable region (VL), wherein the VH comprises the HCDR1, HDR2 and HCDR3 and the VL comprises the LCDR1, LDR2 and LCDR3.

Clause 13: The isolated protein of clause 12, wherein the VH is selected from the group consisting of SEQ ID NOs: 7, 17, 27, 37, 47, 57, 67, 77, 87, 97, 107, 117, 127, and 137 and the VL is selected from the group consisting of SEQ ID NOs: 8, 18, 28, 38, 48, 58, 68, 78, 88, 98, 108, 118, 128, and 138.

Clause 14: The isolated protein of clause 13, wherein the antigen binding domain that binds CD79b comprises
(ww) the VH of SEQ ID NO: 7 and the VL of SEQ ID NO: 8;
(xx) the VH of SEQ ID NO: 17 and the VL of SEQ ID NO: 18
(yy) the VH of SEQ ID NO: 27 and the VL of SEQ ID NO: 28
(zz) the VH of SEQ ID NO: 37 and the VL of SEQ ID NO: 38
(aaa) the VH of SEQ ID NO: 47 and the VL of SEQ ID NO: 48
(bbb) the VH of SEQ ID NO: 57 and the VL of SEQ ID NO: 58
(ccc) the VH of SEQ ID NO: 67 and the VL of SEQ ID NO: 68
(ddd) the VH of SEQ ID NO: 77 and the VL of SEQ ID NO: 78
(eee) the VH of SEQ ID NO: 87 and the VL of SEQ ID NO: 88
(fff) the VH of SEQ ID NO: 97 and the VL of SEQ ID NO: 98;
(ggg) the VH of SEQ ID NO: 107 and the VL of SEQ ID NO: 108;
(hhh) the VH of SEQ ID NO: 117 and the VL of SEQ ID NO: 118;
(iii) the VH of SEQ ID NO: 127 and the VL of SEQ ID NO: 128; or
(jjj) the VH of SEQ ID NO: 137 and the VL of SEQ ID NO: 138.

Clause 15: The isolated protein of any of clauses 1-14, wherein the antigen binding domain that binds CD79b is a scFv, a (scFv)₂, a Fv, a Fab, a F(ab′)₂, a Fd, a dAb or a VHH.

Clause 16: The isolated protein of clause 15, wherein the antigen binding domain that binds CD79b is the Fab.

Clause 17: The isolated protein of clause 15, wherein the antigen binding domain that binds CD79b is the VHH.

Clause 18: The isolated protein of clause 15, wherein the antigen binding domain that binds CD79b is the scFv.

Clause 19: The isolated protein of clause 18, wherein the scFv comprises, from the N- to C-terminus, a VH, a first linker (L1) and a VL (VH-L1-VL) or the VL, the L1 and the VH (VL-L1-VH).

Clause 20: The isolated protein of clause 19, wherein the L1 comprises about 5-50 amino acids, about 5-40 amino acids, about 10-30 amino acids, or about 10-20 amino acids.

Clause 21: The isolated protein of clause 19, wherein L1 comprises an amino acid sequence selected from the group consisting of SEQ ID NOs: 141-173.

Clause 22: The isolated protein of any one of clauses 1-21, wherein the isolated protein is a monospecific protein.

Clause 23: The isolated protein of any one of clauses 1-21, wherein the isolated protein is a multispecific protein.

Clause 24: The isolated protein of clause 23, wherein the multispecific protein is a bispecific protein.

Clause 25: The isolated protein of clause 23, wherein the multispecific protein is a trispecific protein.

Clause 26: The isolated protein of any one of clauses 1-26, further comprising an immunoglobulin (Ig) constant region or a fragment of the Ig constant region thereof.

Clause 27: The isolated protein of any of clauses 12-14, wherein the antigen binding domain that binds CD79b comprises a heavy chain and light chain, wherein the heavy chain comprises the VH and the light chain comprises the VL.

Clause 28: The isolated protein of clause 15, wherein the HC is selected from the group consisting of SEQ ID NOs: 9, 19, 29, 39, 49, 59, 69, 79, 89, 99, 109, 119, 129, or 139 the LC is selected from the group consisting of SEQ ID NOs: 10, 20, 30, 40, 50, 60, 70, 80, 90, 100, 110, 120, 130, or 140.

Clause 29: The isolated protein of clause 13, wherein the antigen binding domain that binds CD79b comprises
(kkk) the HC of SEQ ID NO: 9 and the LC of SEQ ID NO: 10;
(lll) the HC of SEQ ID NO: 19 and the LC of SEQ ID NO: 20;
(mmm) the HC of SEQ ID NO: 29 and the LC of SEQ ID NO: 30;
(nnn) the HC of SEQ ID NO: 39 and the LC of SEQ ID NO: 40;
(ooo) the HC of SEQ ID NO: 49 and the LC of SEQ ID NO: 50;
(ppp) the HC of SEQ ID NO: 59 and the LC of SEQ ID NO: 60;
(qqq) the HC of SEQ ID NO: 69 and the LC of SEQ ID NO: 70;
(rrr) the HC of SEQ ID NO: 79 and the LC of SEQ ID NO: 80;
(sss) the HC of SEQ ID NO: 89 and the LC of SEQ ID NO: 90;
(ttt) the HC of SEQ ID NO: 99 and the LC of SEQ ID NO: 100;
(uuu) the HC of SEQ ID NO: 109 and the LC of SEQ ID NO: 110;
(vvv) the HC of SEQ ID NO: 119 and the LC of SEQ ID NO: 120;
(www) the HC of SEQ ID NO: 129 and the LC of SEQ ID NO: 130; or
(xxx) the HC of SEQ ID NO: 139 and the LC of SEQ ID NO: 140.

Clause 30: An immunoconjugate comprising the isolated protein of any one of clauses 1-29 conjugated to a therapeutic agent or an imaging agent.

Clause 31: A pharmaceutical composition comprising the isolated protein of any one of clauses 1-29 and a pharmaceutically acceptable carrier.

Clause 32: A polynucleotide encoding the isolated protein of any one of clauses 1-29.

Clause 33: A vector comprising the polynucleotide of clause 32.

Clause 34: A host cell comprising the vector of clause 33.

Clause 35: A method of producing the isolated protein of any one of clauses 1-29, comprising culturing the host cell of clause 34 in conditions that the protein is expressed, and recovering the protein produced by the host cell.

Clause 36: A method of administering a therapeutically effective amount of the antigen binding domain that binds CD79b of the isolated protein of any one of clauses 1-29, the immunoconjugate of clause 30, or the pharmaceutical composition of clause 31 to a subject having an autoimmune disease.

Clause 37: A method, comprising administering a therapeutically effective amount of the isolated protein of any one of clauses 1-29, the immunoconjugate of clause 30, or the pharmaceutical composition of clause 31 to a subject having an autoimmune disease.

Clause 38: A method of treating an autoimmune disease in a subject, comprising administering a therapeutically effective amount of the isolated protein of any one of clauses 1-29, the immunoconjugate of clause 30, or the pharmaceutical composition of clause 31 to the subject for a time sufficient to treat the autoimmune disease.

Clause 39: A method of preventing an autoimmune disease in a subject, comprising administering a therapeutically effective amount of the isolated protein of any one of clauses 1-29, the immunoconjugate of clause 30, or the pharmaceutical composition of clause 31 to the subject for a time sufficient to prevent the autoimmune disease.

Clause 40: The method of any of clauses 36-39, wherein the autoimmune disease is associated with or characterized by dysregulation of B cells, autoreactive B cells, or the presence of autoantibodies.

Clause 41: The method of any of clauses 36-40, wherein the autoimmune disease is selected from the group consisting of Systemic lupus erythematosus (SLE), Sjögren's syndrome (SjS), Rheumatoid arthritis, Autoimmune myopathies, Type I diabetes, Addison disease, Pernicious anemia, Autoimmune hepatitis, Primary biliary cholangitis (PBC), Autoimmune pancreatitis, Celiac disease, Focal segmental glomerulosclerosis, Primary membranous nephropathy, Ovarian insufficiency, Autoimmune orchitis, Dry eye disease, Idiopathic interstitial pneumonias, Thyroid disease (eg Grave's), Systemic sclerosis (Scleroderma), Myasthenic syndromes, Autoimmune encephalitis, Bullous skin diseases, TTP, ITP, AIHA, Anca vasculitis, Myocarditis/dilatory CM, NMOSD, Maternal-fetal alloimmunity, Maternal-fetal autoimmunity, Anti-cardiolipin/antiphospholipid syndrome, Hypergammaglobulinemia, Transplant-associated ID, Multifocal motor neuropathy.

Clause 42: A method of modulating B cell activation in a subject, comprising administering a therapeutically effective amount of the isolated protein of any one of clauses 1-29, the immunoconjugate of clause 30, or the pharmaceutical composition of clause 31 to the subject for a time sufficient to modulate B cell activation.

Clause 43: A method of inhibiting aberrant B cell activation in a subject, comprising administering a therapeutically effective amount of the isolated protein of any one of clauses 1-29, the immunoconjugate of clause 30, or the pharmaceutical composition of clause 31 to the subject for a time sufficient to inhibit aberrant B cell activation.

Clause 44: A kit comprising the isolated protein of any one of clauses 1-29, the immunoconjugate of clause 30, or the pharmaceutical composition of clause 31.

Clause 45: An anti-idiotypic antibody binding to the isolated protein of any one of clauses 1-29.

1. An isolated protein comprising an antigen binding domain that binds Cluster of Differentiation 79B protein (CD79b), wherein the antigen binding domain that binds CD79b comprises at least one set of CDRs selected from the group consisting of:
(a) a HCDR1 of SEQ ID NO: 1, a HCDR2 of SEQ ID NO: 2, and a HCDR3 of SEQ ID NO: 3;
(b) a HCDR1 of SEQ ID NO: 11, a HCDR2 of SEQ ID NO: 12, and a HCDR3 of SEQ ID NO: 13;
(c) a HCDR1 of SEQ ID NO: 21, a HCDR2 of SEQ ID NO: 22, and a HCDR3 of SEQ ID NO: 23;
(d) a HCDR1 of SEQ ID NO: 31, a HCDR2 of SEQ ID NO: 32, and a HCDR3 of SEQ ID NO: 33;
(e) a HCDR1 of SEQ ID NO: 41, a HCDR2 of SEQ ID NO: 42, and a HCDR3 of SEQ ID NO: 43;
(f) a HCDR1 of SEQ ID NO: 51, a HCDR2 of SEQ ID NO: 52, and a HCDR3 of SEQ ID NO: 53;
(g) a HCDR1 of SEQ ID NO: 61, a HCDR2 of SEQ ID NO: 62, and a HCDR3 of SEQ ID NO: 63;
(h) a HCDR1 of SEQ ID NO: 71, a HCDR2 of SEQ ID NO: 72, and a HCDR3 of SEQ ID NO: 73;
(i) a HCDR1 of SEQ ID NO: 81, a HCDR2 of SEQ ID NO: 82, and a HCDR3 of SEQ ID NO: 83;
(j) a HCDR1 of SEQ ID NO: 91, a HCDR2 of SEQ ID NO: 92, and a HCDR3 of SEQ ID NO: 93;
(k) a HCDR1 of SEQ ID NO: 101, a HCDR2 of SEQ ID NO: 102, and a HCDR3 of SEQ ID NO: 103;
(l) a HCDR1 of SEQ ID NO: 111, a HCDR2 of SEQ ID NO: 112, and a HCDR3 of SEQ ID NO: 113;
(m) a HCDR1 of SEQ ID NO: 121, a HCDR2 of SEQ ID NO: 122, and a HCDR3 of SEQ ID NO: 123; and
(n) a HCDR1 of SEQ ID NO: 131, a HCDR2 of SEQ ID NO: 132, and a HCDR3 of SEQ ID NO: 133.

2. The isolated protein of claim 1, wherein the antigen binding domain that binds CD79b further comprises at least one set of CDRs selected from the group consisting of:
(a) a LCDR1 of SEQ ID NO: 4, a LCDR2 of SEQ ID NO:5, and a LCDR3 of SEQ ID NO:6;
(b) a LCDR1 of SEQ ID NO: 14, a LCDR2 of SEQ ID NO:15, and a LCDR3 of SEQ ID NO:16;
(c) a LCDR1 of SEQ ID NO: 24, a LCDR2 of SEQ ID NO:25, and a LCDR3 of SEQ ID NO:26;
(d) a LCDR1 of SEQ ID NO: 34, a LCDR2 of SEQ ID NO:35, and a LCDR3 of SEQ ID NO:36;
(e) a LCDR1 of SEQ ID NO: 44, a LCDR2 of SEQ ID NO:45, and a LCDR3 of SEQ ID NO:46;
(f) a LCDR1 of SEQ ID NO: 54, a LCDR2 of SEQ ID NO:55, and a LCDR3 of SEQ ID NO:56;
(g) a LCDR1 of SEQ ID NO: 64, a LCDR2 of SEQ ID NO:65, and a LCDR3 of SEQ ID NO:66;
(h) a LCDR1 of SEQ ID NO: 74, a LCDR2 of SEQ ID NO:75, and a LCDR3 of SEQ ID NO:76;
(i) a LCDR1 of SEQ ID NO: 84, a LCDR2 of SEQ ID NO:85, and a LCDR3 of SEQ ID NO:86;
(j) a LCDR1 of SEQ ID NO: 94, a LCDR2 of SEQ ID NO:95, and a LCDR3 of SEQ ID NO:96;
(k) a LCDR1 of SEQ ID NO: 104, a LCDR2 of SEQ ID NO:105, and a LCDR3 of SEQ ID NO:106;
(1) a LCDR1 of SEQ ID NO: 114, a LCDR2 of SEQ ID NO: 115, and a LCDR3 of SEQ ID NO:116;
(m) a LCDR1 of SEQ ID NO: 124, a LCDR2 of SEQ ID NO:125, and a LCDR3 of SEQ ID NO:126; and
(n) a LCDR1 of SEQ ID NO: 134, a LCDR2 of SEQ ID NO:135, and a LCDR3 of SEQ ID NO:136.

3. The isolated protein of claim 1, wherein the antigen binding domain that binds CD79b comprises at least one set of CDRs selected from the group consisting of:
(a) a HCDR1 of SEQ ID NO: 1, a HCDR2 of SEQ ID NO: 2, a HCDR3 of SEQ ID NO: 3; a LCDR1 of SEQ ID NO: 4, a LCDR2 of SEQ ID NO:5, and a LCDR3 of SEQ ID NO:6;
(b) a HCDR1 of SEQ ID NO: 11, a HCDR2 of SEQ ID NO: 12, a HCDR3 of SEQ ID NO: 13; a LCDR1 of SEQ ID NO: 14, a LCDR2 of SEQ ID NO:15, and a LCDR3 of SEQ ID NO:16;
(c) a HCDR1 of SEQ ID NO: 21, a HCDR2 of SEQ ID NO: 22, a HCDR3 of SEQ ID NO: 23; a LCDR1 of SEQ ID NO: 24, a LCDR2 of SEQ ID NO:25, and a LCDR3 of SEQ ID NO:26;
(d) a HCDR1 of SEQ ID NO: 31, a HCDR2 of SEQ ID NO: 32, a HCDR3 of SEQ ID NO: 33; a LCDR1 of SEQ ID NO: 34, a LCDR2 of SEQ ID NO:35, and a LCDR3 of SEQ ID NO:36;
(e) a HCDR1 of SEQ ID NO: 41, a HCDR2 of SEQ ID NO: 42, a HCDR3 of SEQ ID NO: 43; a LCDR1 of SEQ ID NO: 44, a LCDR2 of SEQ ID NO:45, and a LCDR3 of SEQ ID NO:46;
(f) a HCDR1 of SEQ ID NO: 51, a HCDR2 of SEQ ID NO: 52, a HCDR3 of SEQ ID NO: 53; a LCDR1 of SEQ ID NO: 54, a LCDR2 of SEQ ID NO:55, and a LCDR3 of SEQ ID NO:56;
(g) a HCDR1 of SEQ ID NO: 61, a HCDR2 of SEQ ID NO: 62, a HCDR3 of SEQ ID NO: 63; a LCDR1 of SEQ ID NO: 64, a LCDR2 of SEQ ID NO:65, and a LCDR3 of SEQ ID NO:66;
(h) a HCDR1 of SEQ ID NO: 71, a HCDR2 of SEQ ID NO: 72, a HCDR3 of SEQ ID NO: 73; a LCDR1 of SEQ ID NO: 74, a LCDR2 of SEQ ID NO:75, and a LCDR3 of SEQ ID NO:76;
(i) a HCDR1 of SEQ ID NO: 81, a HCDR2 of SEQ ID NO: 82, a HCDR3 of SEQ ID NO: 83; a LCDR1 of SEQ ID NO: 84, a LCDR2 of SEQ ID NO:85, and a LCDR3 of SEQ ID NO:86;
(j) a HCDR1 of SEQ ID NO: 91, a HCDR2 of SEQ ID NO: 92, a HCDR3 of SEQ ID NO: 93; a LCDR1 of SEQ ID NO: 94, a LCDR2 of SEQ ID NO:95, and a LCDR3 of SEQ ID NO:96;
(k) a HCDR1 of SEQ ID NO: 101, a HCDR2 of SEQ ID NO: 102, a HCDR3 of SEQ ID NO: 103; a LCDR1 of SEQ ID NO: 104, a LCDR2 of SEQ ID NO:105, and a LCDR3 of SEQ ID NO:106;
(l) a HCDR1 of SEQ ID NO: 111, a HCDR2 of SEQ ID NO: 112, a HCDR3 of SEQ ID NO: 113; a LCDR1 of SEQ ID NO: 114, a LCDR2 of SEQ ID NO:115, and a LCDR3 of SEQ ID NO: 116;
(m) a HCDR1 of SEQ ID NO: 121, a HCDR2 of SEQ ID NO: 122, a HCDR3 of SEQ ID NO: 123; a LCDR1 of SEQ ID NO: 124, a LCDR2 of SEQ ID NO:125, and a LCDR3 of SEQ ID NO:126; or
(n) a HCDR1 of SEQ ID NO: 131, a HCDR2 of SEQ ID NO: 132, a HCDR3 of SEQ ID NO: 133; a LCDR1 of SEQ ID NO: 134, a LCDR2 of SEQ ID NO:135, and a LCDR3 of SEQ ID NO:136.

4. The isolated protein of claim 1, wherein the antigen binding domain that binds CD79b comprises a heavy chain variable region (VH), wherein the VH comprises at least one sequence selected from the group consisting of SEQ ID NO: 7, 17, 27, 37, 47, 57, 67, 77, 87, 97, 107, 117, 127, and 137.

5. The isolated protein of claim 1, wherein the antigen binding domain that binds CD79b comprises a light chain variable region (VL), wherein the VL comprises at least one sequence selected from the group consisting of SEQ ID NO: 8, 18, 28, 38, 48, 58, 68, 78, 88, 98, 108, 118, 128, and 138.

6. The isolated protein of claim 1, wherein the antigen binding domain that binds CD79b comprises a VH and VL sequence selected from the group consisting of:
(a) the VH of SEQ ID NO: 7 and the VL of SEQ ID NO: 8;
(b) the VH of SEQ ID NO: 17 and the VL of SEQ ID NO: 18
(c) the VH of SEQ ID NO: 27 and the VL of SEQ ID NO: 28
(d) the VH of SEQ ID NO: 37 and the VL of SEQ ID NO: 38
(e) the VH of SEQ ID NO: 47 and the VL of SEQ ID NO: 48
(f) the VH of SEQ ID NO: 57 and the VL of SEQ ID NO: 58
(g) the VH of SEQ ID NO: 67 and the VL of SEQ ID NO: 68
(h) the VH of SEQ ID NO: 77 and the VL of SEQ ID NO: 78
(i) the VH of SEQ ID NO: 87 and the VL of SEQ ID NO: 88
(j) the VH of SEQ ID NO: 97 and the VL of SEQ ID NO: 98;
(k) the VH of SEQ ID NO: 107 and the VL of SEQ ID NO: 108;
(l) the VH of SEQ ID NO: 117 and the VL of SEQ ID NO: 118;
(m) the VH of SEQ ID NO: 127 and the VL of SEQ ID NO: 128; and
(n) the VH of SEQ ID NO: 137 and the VL of SEQ ID NO: 138.

7. The isolated protein of claim 1, wherein the antigen binding domain that binds CD79b is selected from the group consisting of a scFv, a (scFv)₂, a Fv, a Fab, a F(ab′)2, a Fd, a dAb and a VHH.

8. The isolated protein of claim 7, wherein antigen binding domain that binds CD79b is an scFv, and further wherein the scFv comprises, from the N- to C-terminus, a VH, a first linker (L1) and a VL (VH-L1-VL) or the VL, the L1 and the VH (VL-L1-VH), and further wherein the L1 comprises an amino acid sequence selected from the group consisting of SEQ ID NOs: 141-173.

9. The isolated protein of claim 1, wherein the isolated protein is selected from the group consisting of a monospecific protein, a bispecific protein, a tri-specific protein, and a multi-specific protein.

10. The isolated protein of claim 1, further comprising an immunoglobulin (Ig) constant region or a fragment of the Ig constant region thereof.

11. The isolated protein of claim 1, wherein the antigen binding domain that binds CD79b comprises a heavy chain and light chain, wherein the heavy chain comprises the VH and the light chain comprises the VL, wherein the HC is selected from the group consisting of SEQ ID NOs: 9, 19, 29, 39, 49, 59, 69, 79, 89, 99, 109, 119, 129, or 139 the LC is selected from the group consisting of SEQ ID NOs: 10, 20, 30, 40, 50, 60, 70, 80, 90, 100, 110, 120, 130, or 140.

12. The isolated protein of claim 11, wherein the antigen binding domain that binds CD79b comprises a HC and LC sequence selected from the group consisting of:
(a) the HC of SEQ ID NO: 9 and the LC of SEQ ID NO: 10;
(b) the HC of SEQ ID NO: 19 and the LC of SEQ ID NO: 20;
(c) the HC of SEQ ID NO: 29 and the LC of SEQ ID NO: 30;
(d) the HC of SEQ ID NO: 39 and the LC of SEQ ID NO: 40;
(e) the HC of SEQ ID NO: 49 and the LC of SEQ ID NO: 50;
(f) the HC of SEQ ID NO: 59 and the LC of SEQ ID NO: 60;
(g) the HC of SEQ ID NO: 69 and the LC of SEQ ID NO: 70;
(h) the HC of SEQ ID NO: 79 and the LC of SEQ ID NO: 80;
(i) the HC of SEQ ID NO: 89 and the LC of SEQ ID NO: 90;
(i) the HC of SEQ ID NO: 99 and the LC of SEQ ID NO: 100;
(k) the HC of SEQ ID NO: 109 and the LC of SEQ ID NO: 110;
(l) the HC of SEQ ID NO: 119 and the LC of SEQ ID NO: 120;
(m) the HC of SEQ ID NO: 129 and the LC of SEQ ID NO: 130; and
(n) the HC of SEQ ID NO: 139 and the LC of SEQ ID NO: 140.

13. An immunoconjugate comprising the isolated protein of claim 1 conjugated to a therapeutic agent or an imaging agent.

14. A pharmaceutical composition comprising the isolated protein of claim 1 and a pharmaceutically acceptable carrier.

15. A polynucleotide encoding the isolated protein of claim 1.

16. A vector comprising the polynucleotide of claim 15.

17. A host cell comprising the vector of claim 16.

18. A method of treating or preventing an autoimmune disease in a subject, comprising administering a therapeutically effective amount of the isolated protein of claim 1, to the subject for a time sufficient to treat the autoimmune disease.

19. The method claim 18, wherein the autoimmune disease is selected from the group consisting of Systemic lupus erythematosus (SLE), Sjögren's syndrome (SjS), Rheumatoid arthritis, Autoimmune myopathies, Type I diabetes, Addison disease, Pernicious anemia, Autoimmune hepatitis, Primary biliary cholangitis (PBC), Autoimmune pancreatitis, Celiac disease, Focal segmental glomerulosclerosis, Primary membranous nephropathy, Ovarian insufficiency, Autoimmune orchitis, Dry eye disease, Idiopathic interstitial pneumonias, Thyroid disease (eg Grave's), Systemic sclerosis (Scleroderma), Myasthenic syndromes, Autoimmune encephalitis, Bullous skin diseases, TTP, ITP, AIHA, Anca vasculitis, Myocarditis/dilatory CM, NMOSD, Maternal-fetal alloimmunity, Maternal-fetal autoimmunity, Anti-cardiolipin/antiphospholipid syndrome, Hypergammaglobulinemia, Transplant-associated ID, Multifocal motor neuropathy.

20. A method of modulating B cell activation or inhibiting aberrant B cell activation in a subject, comprising administering a therapeutically effective amount of the isolated protein of claim 1 to the subject for a time sufficient to modulate B cell activation.
